(19) United States
(12) Patent Application Publication
(10) Pub. No.: US 2013/0158112 A1
(43) Pub. Date: Jun. 20, 2013

(54) COMPOSITIONS AND METHODS FOR INHIBITING BETA AMYLOID SECRETION

(71) Applicants: Jonathan D. Smith, Shaker Heights, OH (US); Enakshi Chakrabarti, Westbury, NY (US); Subrata Ghosh, Himachal Pradesh (IN); Marti Sayre, South Euclid, OH (US); Gregory Tochtrop, Shaker Heights, OH (US)

(72) Inventors: Jonathan D. Smith, Shaker Heights, OH (US); Enakshi Chakrabarti, Westbury, NY (US); Subrata Ghosh, Himachal Pradesh (IN); Marti Sayre, South Euclid, OH (US); Gregory Tochtrop, Shaker Heights, OH (US)

(21) Appl. No.: 13/527,844

(22) Filed: Jun. 20, 2012

Related U.S. Application Data
(60) Provisional application No. 61/498,847, filed on Jun. 20, 2011.

(51) Int. Cl. C07C 323/56; C07C 233/69; C07C 323/45; C07C 323/42; C07C 317/28

(52) U.S. Cl. CPC C07C 323/56; C07C 323/42; C07C 317/28; C07C 323/45; C07C 233/69
USPC 514/522; 514/617; 514/562; 514/616; 514/622; 514/619; 514/539

(57) ABSTRACT

A pharmaceutical composition for inhibiting amyloid beta peptide in a subject includes a compound having the formula (I):

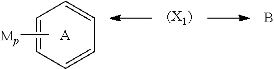

where M is selected from a substituted or unsubstituted alkyl, halo, alkoxy, aryl, cyclic, or heterocyclic group;
p is an integer from 0-3;
X₁ is a 3-9 atoms in length linker connecting A and B;
B is selected from a substituted or unsubstituted aryl, alkoxy or amine group; and
a pharmaceutically acceptable salt thereof; and a pharmaceutical carrier.

### RELATED APPLICATION

This application claims priority from U.S. Provisional Application No. 61/498,847, filed Jun. 20, 2011, the subject matter of which is incorporated herein by reference in its entirety.

### TECHNICAL FIELD

This application relates to β-Amyloid secretion inhibitors, pharmaceutical compositions comprising the same, and methods for formulating or using the same to treat diseases or disorders associated with β-Amyloid secretion.

### BACKGROUND OF THE INVENTION

Alzheimer's disease (AD) is the most prevalent cause of dementia in the United States. The incidence of AD increases dramatically with aging, and it is estimated to affect almost half of the individuals over 85 years of age. As the baby-boom generation is aging, both the number of individuals and the proportion of the population at risk for AD is expanding. The burden of AD falls not only on the individuals who eventually loses all cognitive function, but on the families that bear the emotional and financial stress, and upon society that must support both the patients and their families. Currently there are no drugs that can cure AD or even halt the progression of dementia. FDA approved drugs for dementia only afford a small delay in disease progression. Thus, there remains a need for pharmaceutical compositions capable of preventing or delaying the onset of AD by inhibiting the cellular production of amyloid β peptide.

### SUMMARY OF THE INVENTION

This application relates to β-Amyloid secretion inhibitors, pharmaceutical compositions comprising the same, and methods for formulating or using the same to treat diseases or disorders associated with β-Amyloid secretion. One aspect of the application relates to a pharmaceutical composition for inhibiting β-Amyloid secretion in a subject. The pharmaceutical composition can include a compound having the formula (I):

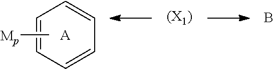

where M is a substituted or unsubstituted alkyl, halo, alkoxy, aryl, cyclic, or heterocyclic group; p is an integer from 0-3; X₁ is a 3-9 atoms in length linker connecting A and B; B is selected from a substituted or unsubstituted aryl, alkoxy or amine group; and a pharmaceutically acceptable salts thereof.

Another aspect of the application relates to a method for inhibiting β-Amyloid secretion in a subject. The method can comprise administering to the subject a therapeutically effective amount of a pharmaceutical composition including a compound having the formula (I):

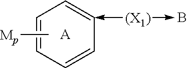

where M is a substituted or unsubstituted alkyl, halo, alkoxy, aryl, cyclic, or heterocyclic group; p is an integer from 0-3; X₁ is a 3-9 atoms in length linker connecting A and B; B is selected from a substituted or unsubstituted aryl, alkoxy or amine group; and a pharmaceutically acceptable salt thereof.

A further aspect of the application relates to a method treating a neurological or neurodegenerative disorder in a subject. The method can include administering to the subject a therapeutically effective amount of a pharmaceutical composition including a compound having the formula (I):

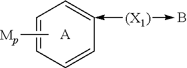

where M is a substituted or unsubstituted alkyl, halo, alkoxy, aryl, cyclic, or heterocyclic group; p is an integer from 0-3; X₁ is a 3-9 atoms in length linker connecting A and B; B is selected from a substituted or unsubstituted aryl, alkoxy or amine group; and a pharmaceutically acceptable salt thereof. In some aspects of the application, the neurological or neurodegenerative disorder is Alzheimer's disease. In other aspects, the neurological or neurodegenerative disorder can include a central or peripheral nervous system injury, such as traumatic brain injury (TBI), stroke, or multiple sclerosis, and the pharmaceutical composition can be administered to the subject prior to or immediately after onset of the central nervous system injury.

### BRIEF DESCRIPTION OF THE DRAWINGS

The foregoing and other features of the application will become apparent to those skilled in the art to which the application relates upon reading the following description with reference to the accompanying drawings, in which:

FIG. 1(A) illustrates the chemical structure of a compound in accordance with an aspect of the application (compound 1) with the labeling of two phenyl rings; (B) is a graph of the dose response of compound 1 on Aβ secretion and total cellular protein after 24 h incubation with H4βPAPP695 wt cells or (C) primary neurons from APP/PS1 transgenic mice.

FIG. 2 (A) is a schematic illustration showing APP processing and Western blot hands from conditioned media (top) and cell lysate (bottom) after 24 h treatment with 10 μM compound 1 or vehicle control. The cleavage sites of the α, β and γ secretases are shown. Compound 1 treatment leads to increased sAPPα and αCTF without altering holoAPP levels, indicating increased α-secretase cleavage of APP; (B) Quantification of sAPPα induction by 10 μM compound 1 treatment (N=3 each ±SD,***, P<0.0001); (C) Dose response of sAPPα induction by compound 1 showing Western blot band (top) and quantification (bottom).

FIG. 3(A) is a graph showing αCTF induction by compound 1, Quantification of αCTF induction by 24 h treatment with 10 μM compound 1 or vehicle control (N=7 each ±SD, *, p<0.05); (B) Time course of αCTF induction by 10 μM compound 1.

FIG. 4 illustrates α-secretase activity measured in vitro is induced by treatment of cells with compound 1; (A) is a graph showing α-secretase in cell lysates from cells treated for 24 h with 10 μM compound 1 or vehicle control (N=6 each ±SD, *., p<0.001); (B) is a graph showing the dose response of α-secretase activity induction by compound 1.

FIG. 5 is a graph showing the effect of compound 1 on Aβ secretion is not blocked by PKC inhibitors. Results show secreted Aβ levels compared to the respective controls with compound 1 treated cells shown in open bars (N=4 each, ±SO, **, p<0.01; and p<0.001 for effect compound 1 vs. the respective control for each treatment in the filled bar.

FIG. 6 illustrates that compound 1 promotes cellular processing of ADAM 9 and ADAM10. Compound 1 at increasing doses was incubated on H4βPAPP695 wt cells for 24 h. Cell lysates were prepared for Western blots. Arrows point to the pro and active forms of ADAM9, ADAM10, and ADAM17.

FIG. 7 is a graph showing compound 48 decreases cerebral Aβ levels in mice. APP/PS1 transgenic mice were injected iv with 100 to 125 mg/kg compound 48 or vehicle control. 6 hours later, the mice were sacrificed and cerebral Aβ levels were measured by an ELISA assay and normalized to cerebral protein. Compound 48 led to a 39.6% reduction in cerebral Aβ levels (p<0.0001).

FIG. 8 is a schematic illustration showing the synthetic analysis for defining a structure activity relationship for compound 1.

FIG. 9 is a schematic illustration showing the general synthetic scheme for the preparation of compound 1 analogues.

FIG. 10(A-B) illustrates graphs showing body weight and cerebral Aβ levels in mutant APP/PS1 transgenic mice administered compound 48.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the application pertains. Commonly understood definitions of molecular biology terms can be found in, for example, Rieger et al., Glossary of Genetics: Classical and Molecular, 5th Ed., Springer-Verlag: New York, 1991, and Lewin, Genes V, Oxford University Press: New York, 1994. The definitions provided herein are to facilitate understanding of certain terms used frequently herein and are not meant to limit the scope of the application.

As used herein, the term “therapeutically effective amount” can refer to that amount of a pharmaceutical composition that results in amelioration of symptoms or a prolongation of survival in a subject. A therapeutically relevant effect relieves to some extent one or more symptoms of a disease or condition or returns to normal either partially or completely one or more physiological or biochemical parameters associated with or causative of the disease or condition.

As used herein, the terms “treating” or “treatment” of a condition or disease can include: (1) preventing at least one symptom of the disorder, disease or condition, i.e., causing a clinical symptom to not significantly develop in a subject that may develop or be predisposed to the disease but does not yet experience or display symptoms of the disease or condition; (2) inhibiting the disease or condition, i.e., arresting, delaying or reducing the development of the disease or condition and its symptoms; or (3) relieving the disease or condition, i.e., causing regression of the disease or condition and its clinical symptoms.

As used herein, the term “subject” can refer to any animal, including, but not limited to, humans and non-human animals (e.g., rodents, arthropods, insects, fish (e.g., zebrafish)), non-human primates, ovines, bovines, ruminants, lagomorphs, porcines, caprines, equines, canines, felines, ayes, etc.), which is to be the recipient of a particular treatment. Typically, the terms “patient” and “subject” are used interchangeably herein in reference to a human subject.

The term “alkyl” can refer to a branched or unbranched saturated hydrocarbon group typically, although not necessarily, containing 1 to about 24 carbon atoms, such as methyl, ethyl, n-propyl, isopropyl, n-butyl, isobutyl, t-butyl, octyl, decyl, and the like, as well as cycloalkyl groups, such as cyclopentyl, cyclohexyl, and the like. Generally, alkyl groups can contain 1 to about 18 carbon atoms and, for example, 1 to about 12 carbon atoms. The term “lower alkyl” can refer to an alkyl group of 1 to 6 carbon atoms. Substituents identified as “C₁-C₆ alkyl” or “lower alkyl” can contain 1 to 3 carbon atoms and, more particularly, such substituents can contain 1 or 2 carbon atoms (i.e., methyl and ethyl). “Substituted alkyl” can refer to an alkyl substituted with one or more substituent groups. The terms “heteroatom-containing alkyl” and “heteroalkyl” can refer to an alkyl in which at least one carbon atom is replaced with a heteroatom, as described in further detail below. If not otherwise indicated, the terms “alkyl” and “lower alkyl” can include linear, branched, cyclic, unsubstituted, substituted, and/or heteroatom-containing alkyl or lower alkyls, respectively.

The term “alkenyl” can refer to a linear, branched, or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, cyclopentenyl, cyclohexenyl, cyclooctenyl, and the like. Generally, alkenyl groups can contain 2 to about 18 carbon atoms, and more particularly 2 to 12 carbon atoms. The term “lower alkenyl” can refer to an alkenyl group of 2 to 6 carbon atoms, and the term “cycloalkenyl” can refer to a cyclic alkenyl group having 5 to 8 carbon atoms. The term “substituted alkenyl” can refer to an alkenyl substituted with one or more substituent groups, and the terms “heteroatom-containing alkenyl” and “heteroalkenyl” can refer to an alkenyl or heterocycloalkenyl (e.g., heterocylcohexenyl) in which at least one carbon atom is replaced with a heteroatom. If not otherwise indicated, the terms “alkenyl” and “lower alkenyl” can include linear, branched, cyclic, unsubstituted, substituted, and/or heteroatom-containing alkenyls and lower alkenyls, respectively.

The term “alkynyl” can refer to a linear or branched hydrocarbon group of 2 to 24 carbon atoms containing at least one triple bond, such as ethynyl, n-propynyl, and the like. Generally, alkynyl groups can contain 2 to about 18 carbon atoms, and more particularly 2 to 12 carbon atoms. The term “lower alkynyl” can refer to an alkynyl group of 2 to 6 carbon atoms. The term “substituted alkynyl” can refer to alkynyl substituted with one or more substituent groups, and the terms “heteroatom-containing alkynyl” and “heteroalkynyl” can refer to an alkynyl in which at least one carbon atom is replaced with a heteroatom. If not otherwise indicated, the terms “alkynyl” and “lower alkynyl” can include linear, branched, unsubstituted, substituted, and/or heteroatom-containing alkynyls and lower alkynyls, respectively.

The term “alkoxy” can refer to an alkyl group bound through a single terminal ether linkage; that is, an “alkoxy” group may be represented as —O-alkyl where alkyl is defined as above. A “lower alkoxy” group can refer to an alkoxy group containing 1 to 6 carbon atoms, and includes, for example, methoxy, ethoxy, n-propoxy, isopropoxy, t-butyloxy, etc. Examples of substituents identified as “C₁-C₆ alkoxy” or “lower alkoxy” can contain 1 to 3 carbon atoms, and more particularly 1 or 2 carbon atoms (i.e., methoxy and ethoxy).

The term “aryl” can refer to an aromatic substituent containing a single aromatic ring or multiple aromatic rings that are fused together, directly linked, or indirectly linked (so that the different aromatic rings are bound to a common group, such as a methylene or ethylene moiety). Aryl groups can contain 5 to 20 carbon atoms, and more particularly 5 to 14 carbon atoms. Exemplary aryl groups can contain one aromatic ring or two fused or linked aromatic rings, e.g., phenyl, naphthyl, biphenyl, diphenylether, diphenylamine, benzophenone, and the like. The aryl can be a substituted aryl, heteroatom-containing aryl, heteroaryl, “Substituted aryl” can refer to an aryl moiety substituted with one or more substituent groups, and the terms “heteroatom-containing aryl” and “heteroaryl” can refer to an aryl group in which at least one carbon atom is replaced with a heteroatom, as will be described in further detail below. If not otherwise indicated, the term “aryl” can include unsubstituted, substituted, and/or heteroatom-containing aromatic substituents.

The term “aryloxy” can refer to an aryl group bound through a single terminal ether linkage, wherein “aryl” can be defined above. An “aryloxy” group may be represented as -D-aryl, where “aryl” can be defined above. Aryloxy groups can contain 5 to about 20 carbon atoms, and more particularly 5 to 14 carbon atoms. Examples of aryloxy groups can include, without limitation, phenoxy, o-halo-phenoxy, m-halo-phenoxy, p-halo-phenoxy, o-methoxy-phenoxy, m-methoxy-phenoxy, p-methoxy-phenoxy, 2,4-dimethoxy-phenoxy, 3,4,5-trimethoxy-phenoxy, and the like.

The term “alkaryl” can refer to an aryl group with an alkyl substituent, and the term “aralkyl” can refer to an alkyl group with an aryl substituent, wherein “aryl” and “alkyl” can be defined as above. Exemplary aralkyl groups can contain 6 to 24 carbon atoms, and more particularly 6 to 16 carbon atoms. Examples of aralkyl groups can include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. Alkaryl groups include, for example, p-methylphenyl, 2,4-dimethylphenyl, p-cyclohexylphenyl, 2,7-dimethylnaphthyl, 7-cyclooctylnaphthyl, 3-ethyl-cyclopenta-1,4-diene, and the like.

The term “cyclic” can refer to alicyclic or aromatic substituents that may or may not be substituted and/or heteroatom containing, and that may be monocyclic, bicyclic, or polycyclic.

The terms “halo” and “halogen” can be used in the conventional sense and refer to a chloro, bromo, fluoro or iodo substituent.

The term “heteroatom-containing” as in a “heteroatom-containing alkyl group” (also termed a “heteroalkyl” group) or a “heteroatom-containing aryl group” (also termed a “heteroaryl” group) can refer to a molecule, linkage, or substituent in which one or more carbon atoms are replaced with an atom other than carbon, e.g., nitrogen, oxygen, sulfur, phosphorus or silicon, nitrogen, oxygen or sulfur. Similarly, the term “heteroalkyl” can refer to an alkyl substituent that is heteroatom-containing. The term “heterocyclic” can refer to a cyclic substituent that is heteroatom-containing, and the terms “heteroaryl” and “heteroaromatic” can respectively refer to “aryl” and “aromatic” substituents that are heteroatom-containing. Examples of heteroalkyl groups can include alkoxyaryl, alkylsulfanyl-substituted alkyl, N-alkylated amino alkyl, and the like. Examples of heteroaryl substituents can include pyrrolyl, pyrrolidinyl, pyridinyl, quinolinyl, indolyl, pyrimidinyl, imidazolyl, 1,2,4-triazolyl, tetrazolyl, etc. Examples of heteroatom-containing alicyclic groups can include pyrrolidino, morpholino, piperazino, piperidino, etc.

“Hydrocarbyl” can refes to univalent hydrocarbyl radicals containing 1 to about 30 carbon atoms, and more particularly about 1 to 12 carbon atoms, including linear, branched, cyclic, saturated, and unsaturated species, such as alkyl groups, alkenyl groups, aryl groups, and the like. “Substituted hydrocarbyl” can refer to hydrocarbyl substituted with one or more substituent groups, and the term “heteroatom-containing hydrocarbyl” can refer to hydrocarbyl in which at least one carbon atom is replaced with a heteroatom. Unless otherwise indicated, the term “hydrocarbyl” can include substituted and/or heteroatom-containing hydrocarbyl moieties.

By “substituted”, e.g., “substituted alkyl,” “substituted aryl,” and the like, it is meant that in the alkyl, aryl, or other moiety, at least one hydrogen atom bound to a carbon (or other) atom can be replaced with one or more non-hydrogen substituents. Examples of such substituents can include, without limitation, functional groups, such as halo, hydroxyl, silyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO—), carbamoyl (—(CO)—NH₂), mono-(C₁-C₂₄ alkyl)-substituted carbamoyl (—(CO)—NH(C₁-C₂₄ alkyl)), di-(C₁-C₄ alkyl)-substituted carbamoyl (—(CO)—N(C₁-C₂₄ alkyl)₂), mono-substituted arylcarbamoyl (—(CO)—NH-aryl), thiocarbamoyl (—(CS)—NH₂), carbamido (—NH—(CO)—NH₂), cyano(—CN), isocyano (—N C), cyanato (—O—CN), isocyanato (—ON⁺C⁻), isothiocyanato (—S—CN), azido (—N═N⁺═N⁻), formyl (—(CO)—H), thioformyl (—(CS)—H), amino (—NH₂), mono- and di-(C₁-C₂₄ alkyl)-substituted amino, mono- and di-(C₅-C₂₀ aryl)-substituted amino, C₂-C₂₄ alkylamido (—NH—(CO)-alkyl), C₆-C₂₀ arylamido (—NH—(CO)-aryl), imino (—CR═NH where R=hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (-(—(SO)-aryl), C₁-C₂₄ alkylsulfonyl (—SO₂-alkyl), C₅-C₂₀ arylsulfonyl (—SO₂-aryl), phosphono (—P(O)(OH)₂), phosphonato (—P(O)(O⁻)₂), phosphinato (—P(O)(O⁻)), phospho (—PO₂), and phosphino (—PH₂); and the hydrocarbyl moieties C₁-C₂₄ alkyl, C₂-C₂₄ alkenyl, C₂-C₂₄ alkynyl, C₅-C₂₀ aryl, C₆-C₂₄ alkaryl, and C₆-C₂₄ aralkyl.

In addition, the aforementioned functional groups may, if a particular group permits, be further substituted with one or more additional functional groups or with one or more hydrocarbyl moieties, such as those specifically enumerated above. Analogously, the above-mentioned hydrocarbyl moieties may be further substituted with one or more functional groups or additional hydrocarbyl moieties, such as those specifically enumerated.

When the term “substituted” appears prior to a list of possible substituted groups, the term can apply to every member of that group. For example, the phrase “substituted alkyl, alkenyl, and aryl” can refer to “substituted alkyl, substituted alkenyl, and substituted aryl.” Analogously, when the term “heteroatom-containing” appears prior to a list of possible heteroatom-containing groups, the term can apply to every member of that group. For example, the phrase “heteroatom-containing alkyl, alkenyl, and aryl” can refer to “heteroatom-containing alkyl, substituted alkenyl, and substituted aryl.”

“Optional” or “optionally” can mean that the subsequently described circumstance may or may not occur, so that the description includes instances where the circumstance occurs and instances where it does not. For example, the phrase “optionally substituted” can mean that a non-hydrogen substituent may or may not be present on a given atom and, thus, the description can include structures where a non-hydrogen substituent is present and structures where a non-hydrogen substituent is not present.

The phrase “having the formula” or “having the structure” is not intended to be limiting and can be used in the same way that the term “comprising” is commonly used.

The term “analog” can mean a compound in which one or more individual atoms have been replaced, either with a different atom or with a different functional group, and where replacement of the atom does not substantially eliminate or reduce the compound's ability to act as an Aβ secretion inhibitor.

Embodiments of the application described herein relate, at least in part, to compounds that can activate the non-amyloidogenic α-secretase pathway of the amyloid precursor protein (APP) and thereby inhibit β- and γ-secretase cleavage of APP to Aβ. It is believed that the compounds described herein can promote the cellular processing of two proteases with α-secretase activity, ADAM9 and ADAM10. As discussed in more detail below, certain diseases or disorders are mediated by the secretion and aggregation of amyloid β (Aβ), which can result in the formation of senile plaques and cognitive impairment in a subject. The compounds described herein can increase α-secretase processing of APP to a neurotrophic and neuroprotective soluble peptide (sAPPα), consequently, modulating (e.g., decreasing) Aβ secretion by precluding Aβ peptide formation. In certain aspects, the hydrophobic amide compounds can effectively suppress neuronal toxicity induced by amyloid β (Aβ)-oligomers and provide neuroprotective effect to the subject effective to mitigate nervous system injuries.

In some embodiments, the compound can have the following formula (I):

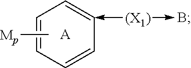

where M is a substituted or unsubstituted alkyl, halo, alkoxy, aryl, cyclic, or heterocyclic group; p is an integer from 0-3; X₁ is a 3-9 atoms in length linker connecting A and B; B is selected from a substituted or unsubstituted aryl, alkoxy or amine group; and pharmaceutically acceptable salts thereof.

In other embodiments, the compound can have the following general formula (II):

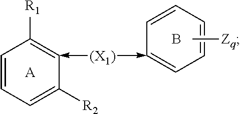

where X₁ is a 6-7 atoms in length linker connecting A and B; q is an integer from 0-3; R₁, R₂, and Z can each independently represent substituents selected from the group consisting of hydrogen, halo, or substituted or unsubstituted C₁-C₂₄ alkyl, C₂-C₂₄ alkenyl, C₂-C₂₄ alkynyl, C₃-C₂₀ aryl, C₆-C₂₄ alkaryl, C₆-C₂₄ aralkyl, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl, C₂-C₂₄ alkylcarbonyl (—CO-alkyl), C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO), carbamoyl (—(CO)—NH₂), mono-(C₁-C₂₄ alkyl)-substituted carbamoyl (—(CO)—NH(Cᵢ C₂₋₄ alkyl)), di-(C₁-C₂₄ alkyl)-substituted carbamoyl (—(CO)—N(C₁-C₂₄ alkyl)₂), mono-substituted arylcarbamoyl (—(CO)—NH-aryl), thiocarbamoyl (—(CS)—NH₂), carbamido (—NH—(CO)—NH₂), cyano(—CN), isocyano (—N⁺C⁻), cyanato (—O—CN), isocyanato (—O—N⁺═C⁻), isothiocyanato (—S—CN), azido (—N═N⁺═N⁻), formyl (—(CO)—H), thioformyl (—(CS)—H), amino (—NH₂), mono- and di-(C₁-C₂₄ alkyl)-substituted amino, mono- and di-(C₅-C₂₀ aryl)-substituted amino, C₂-C₂₄ alkylamido (—NH—(CO)-alkyl), C₆-C₂₀ arylamido (—NH—(CO)-aryl), imino, alkylimino, arylimino, nitro (—NO₂), nitroso (—NO), sulfo (—SO₂₋OH), sulfonato (—SO₂—O⁻), C₁-C₂₄ alkylsulfanyl (—S-alkyl), arylsulfanyl, C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), phosphono (—P(O)(OH)₂), phosphonato (—P(O)(O—)₂), phosphinato (—P(O)(O⁻)), phospho (—PO₂), phosphino (—PH₂), and combinations thereof.

In some embodiments, R₁ and R₂ are individually selected from a substituted or unsubstituted aryl, cyclic, or heterocyclic group; X₁ is a 6-7 atoms in length linker connecting A and B; Z is a substituted or unsubstituted alkyl, halo, alkoxy, or amine group; and q is an integer from 0-3.

In other embodiments, R₁ and R₂ are independently selected from the group consisting of H, Cl, CH₃, OH, NO₂, F, Br, CF₃ and an alkoxy group. In some aspects, Z can be selected from Cl, alkoxy, OH, CN, C(NH₂)NOH, NO₂, NH₂, CO₂Et, COOH, CONH, F, CH₂, CHO, CF₃, BR, I, CONHC₃H₅, NHCOC₃H₅ and OCH₂CH₂OCH₂CH₂OH.

In some embodiments, X₁ can have the following general structure:

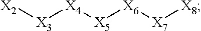

where X₂ is selected from CH₂ and CO; X₃ is selected from NH, CH₂ and CO; X₄ is selected from NH and CH₂; X₅ is CH₂; X₆ is selected from SH₂, SHO, SO₂ and CH₂; where X₇ is selected from SH₂, SHO, SO₂ and CH₂; and where X₈ is selected from nothing and CH₂.

In some embodiments, the compounds can be selected from the group consisting of:

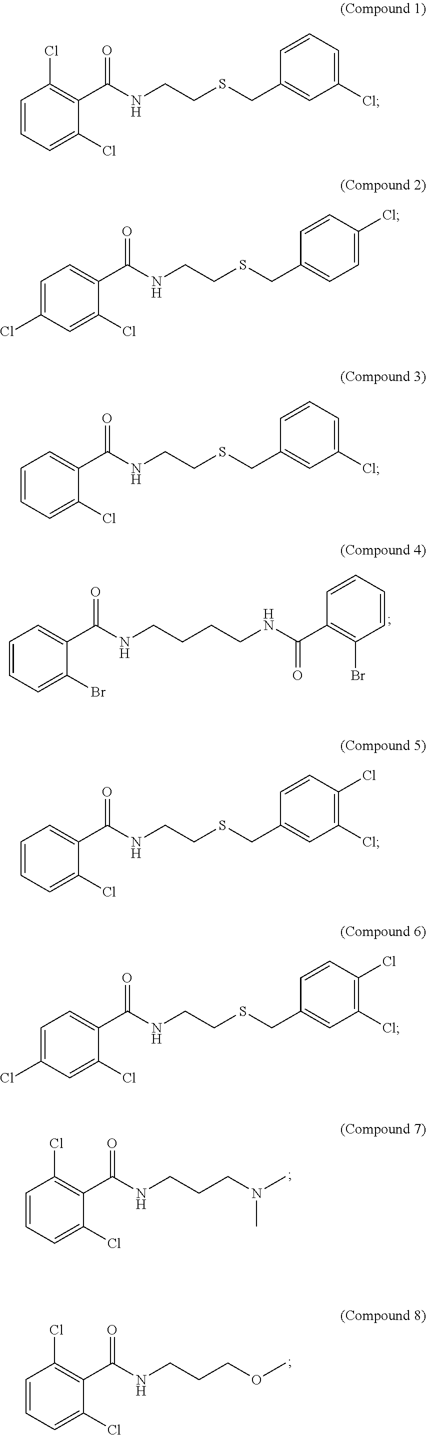

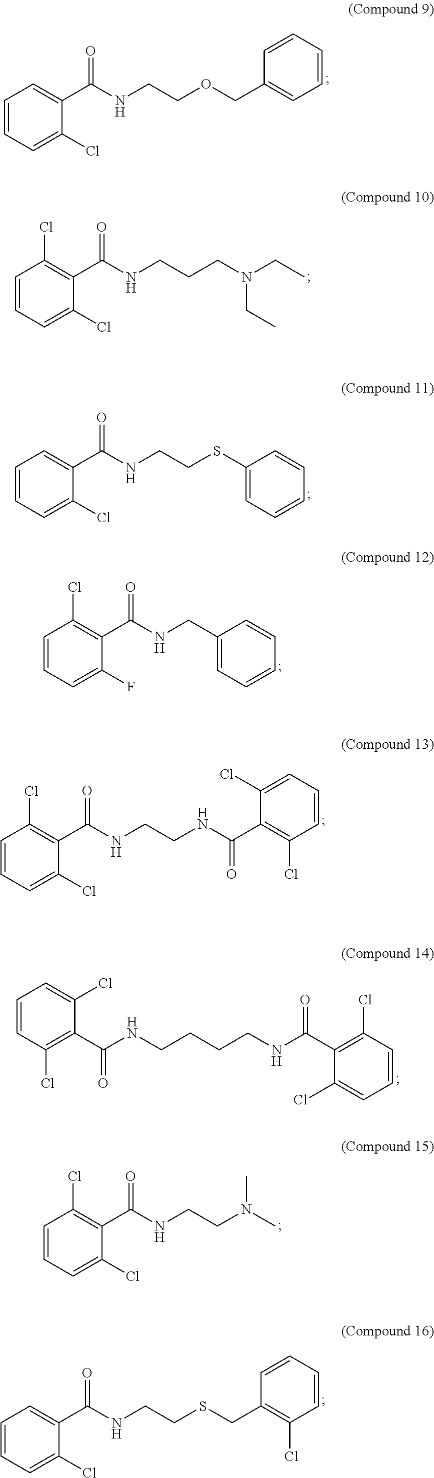

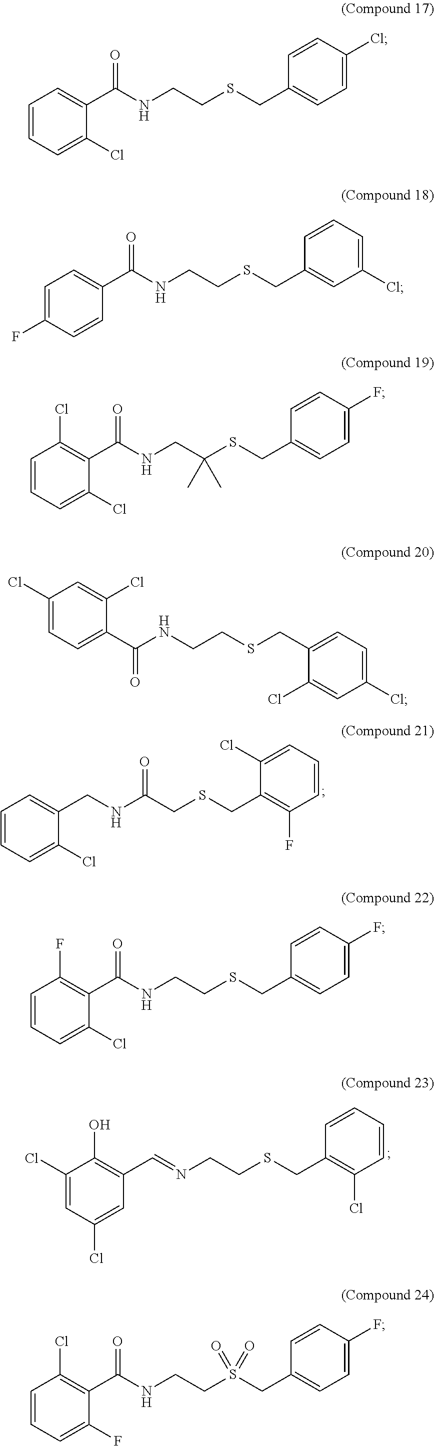

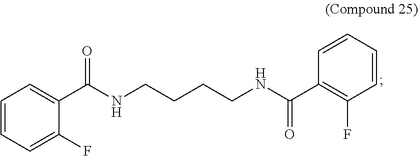 [SMILES: O=C(NCCCCNC(=O)c1ccccc1F)c1ccccc1F]

and pharmaceutically acceptable salts thereof.

In other embodiments, the compounds can be selected from the group consisting of:

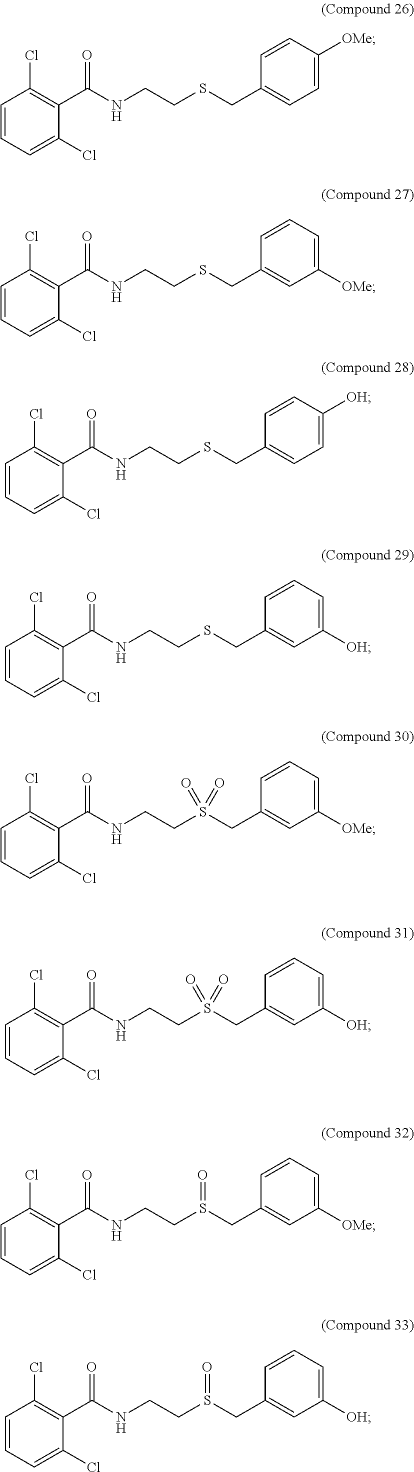

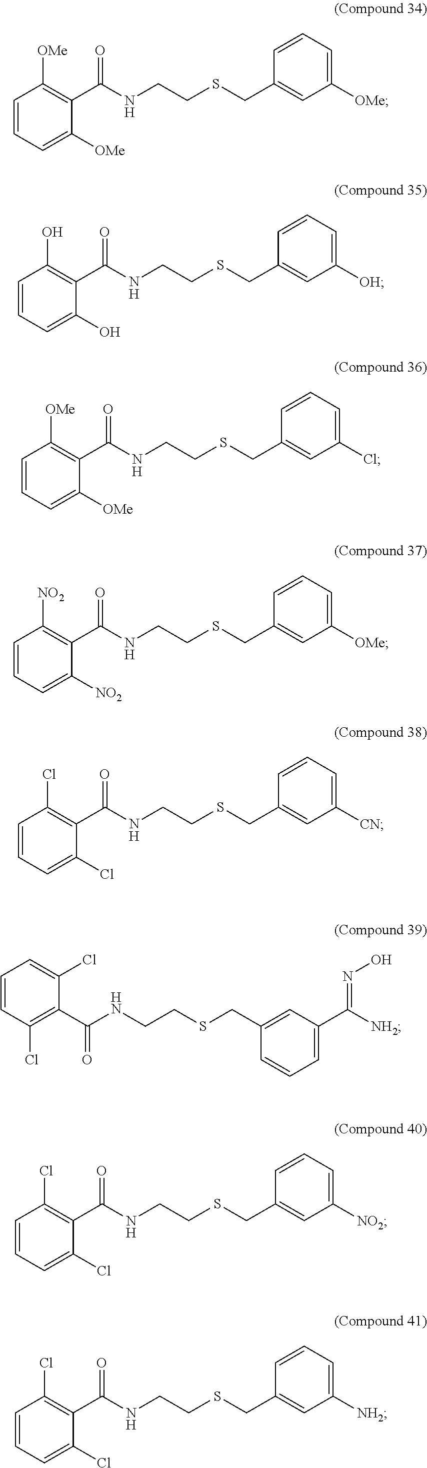

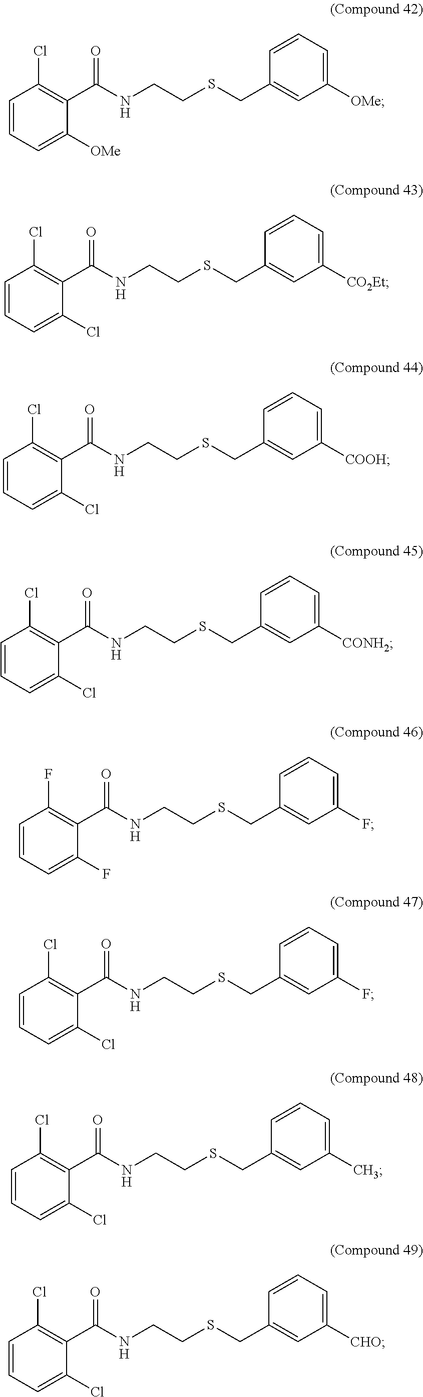

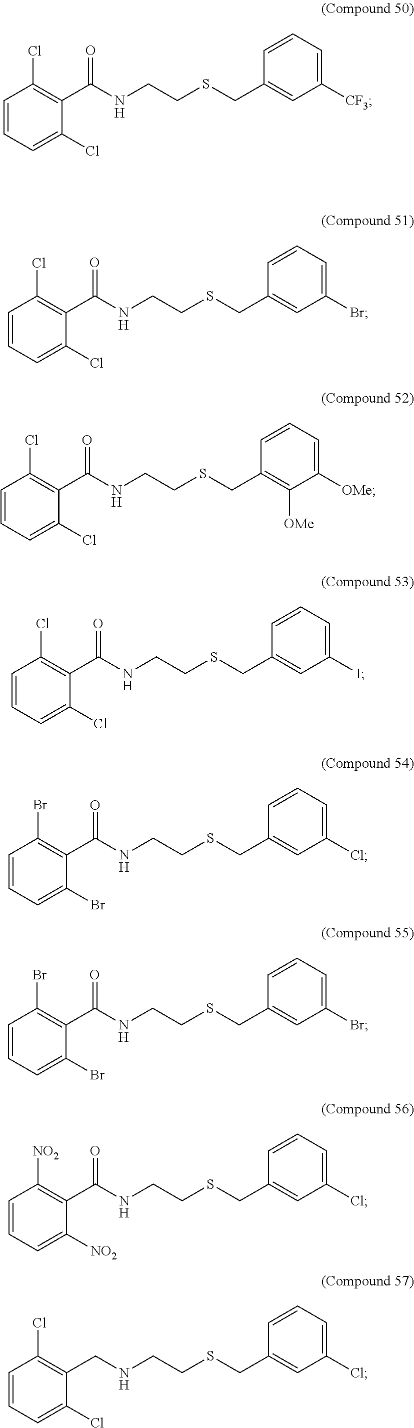

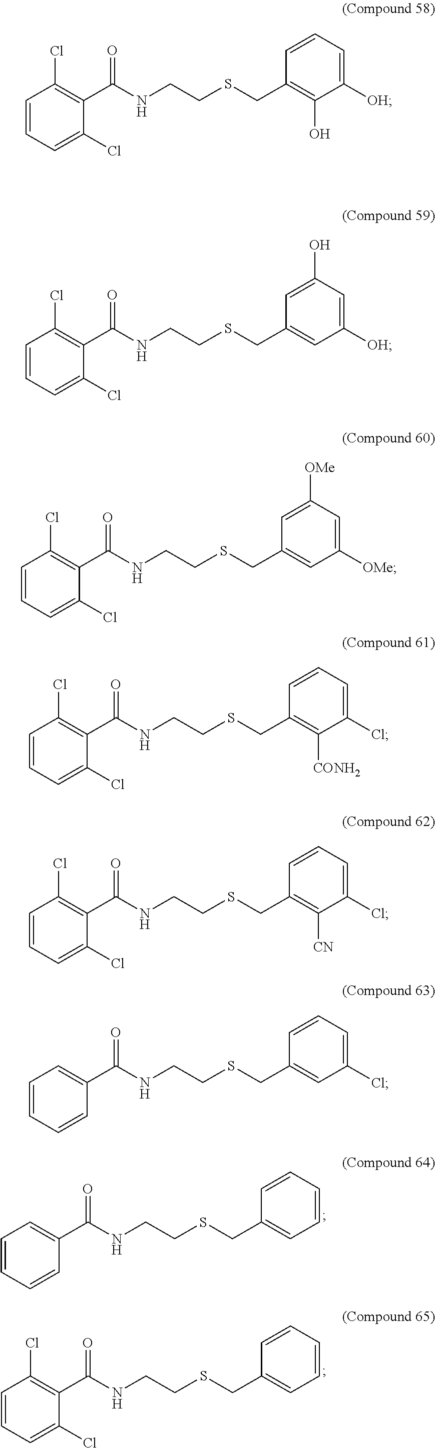

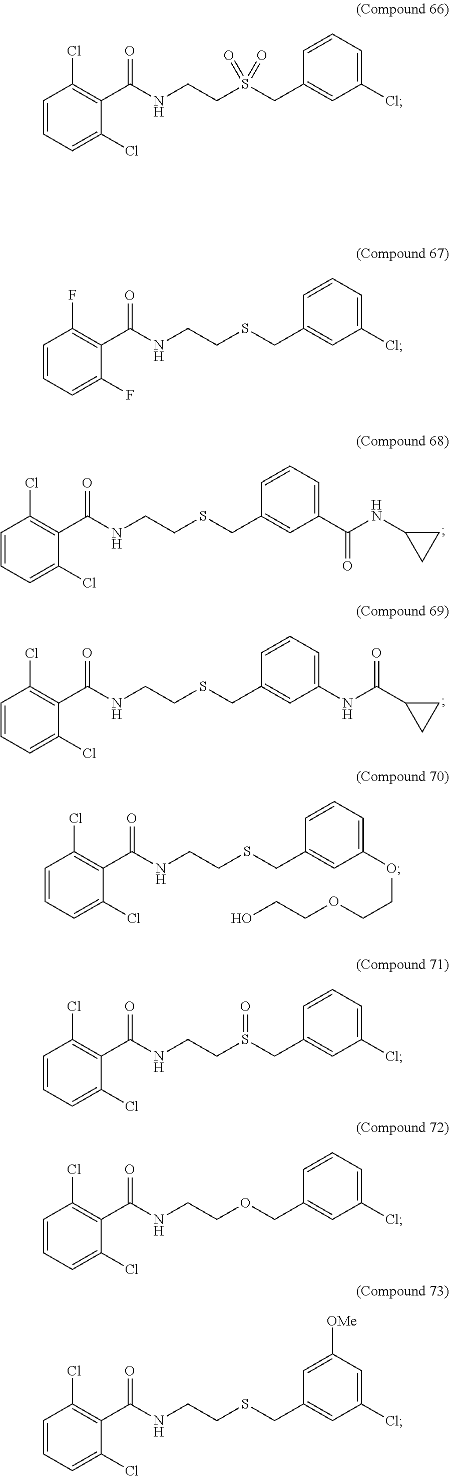

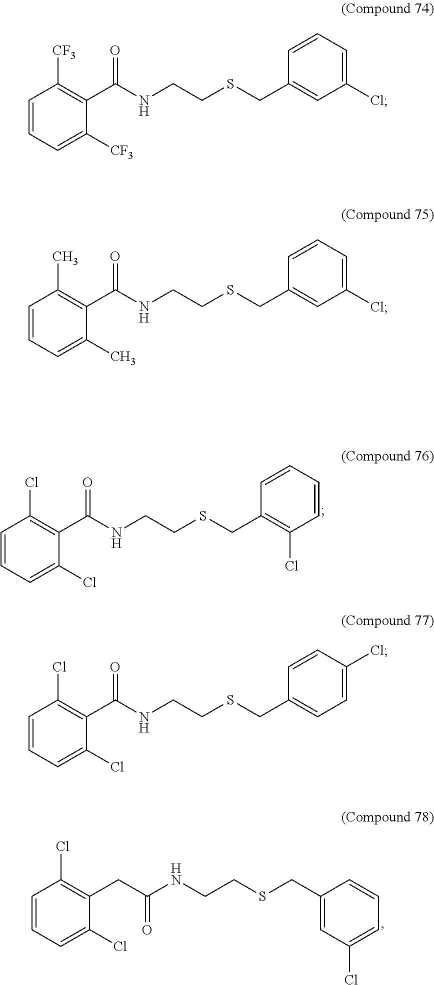 [SMILES: O=C(NCCSCc1cccc(Cl)c1)c1c(C(F)(F)F)cccc1C(F)(F)F ; Cc1cccc(C)c1C(=O)NCCSCc1cccc(Cl)c1 ; O=C(NCCSCc1ccccc1Cl)c1c(Cl)cccc1Cl ; O=C(NCCSCc1ccc(Cl)cc1)c1c(Cl)cccc1Cl ; O=C(Cc1c(Cl)cccc1Cl)NCCSCc1cccc(Cl)c1]

where OMe is a methoxy group; and pharmaceutically acceptable salts thereof.

The compounds described herein can be provided or formulated as a pharmaceutical composition. A pharmaceutical composition including the compound can contain pharmaceutically acceptable carriers, such as excipients and auxiliaries, that facilitate processing of the compounds described herein into compositions that can be used pharmaceutically. The pharmaceutical compositions can be manufactured in a known manner, such as by conventional mixing, granulating, dragee-making, dissolving, lyophilizing processes, and the like. For example, pharmaceutical compositions for oral use can be obtained by combining the hydrophobic amides (or analogs thereof) of the application with solid excipients, optionally grinding the resulting mixture, and processing the mixture of granules after adding auxiliaries (if desired or necessary) to obtain tablets or dragee cores.

Excipients that can be used as part of the pharmaceutical composition can include fillers, such as saccharides (e.g., lactose or sucrose), mannitol or sorbitol, cellulose preparations and/or calcium phosphates, for example, tricalcium phosphate or calcium hydrogen phosphate, as well as binders, such as starch paste using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents can be added, such as the above-mentioned starches and also carboxymethyl-starch, cross-linked polyvinyl pyrrolidone, agar, or alginic acid or a salt thereof, such as sodium alginate. Auxiliaries can include flow-regulating agents and lubricants, such as silica, talc, stearic acid or salts thereof, such as magnesium stearate or calcium stearate, and/or polyethylene glycol. Dragee cores can be provided with coatings that, if desired, are resistant to gastric juices. For this purpose, concentrated saccharide solutions can be used, which may optionally contain gum arabic, talc, polyvinyl pyrrolidone, polyethylene glycol, and/or titanium dioxide, lacquer solutions and suitable organic solvents or solvent mixtures. To produce coatings resistant to gastric juices, solutions of suitable cellulose preparations, such as acetylcellulose phthalate or hydroxypropylmethylcellulose phthalate can be used. Slow-release and prolonged-release formulations may be used with particular excipients, such as methacrylic acid-ethylacrylate copolymers, methacrylic acid-ethyl acrylate copolymers, methacrylic acid-methyl methacrylate copolymers, and methacrylic acid-methyl methylacrylate copolymers. Dye stuffs or pigments can be added to the tablets or dragee coatings, for example, for identification or to characterize combinations of active compound doses.

Other pharmaceutical preparations that can be used orally include push-fit capsules made of gelatin, as well as soft, sealed capsules made of gelatin and a plasticizer, such as glycerol or sorbitol. The push-fit capsules can contain the active compounds in the form of granules that may be mixed with fillers, such as lactose, binders, such as starches, and/or lubricants, such as talc or magnesium stearate and, optionally, stabilizers. In soft capsules, the active compounds can be dissolved or suspended in suitable liquids, such as fatty oils or liquid paraffin. In addition, stabilizers may be added.

Examples of formulations for parenteral administration can include aqueous solutions of the active compounds in water-soluble form, for example, water-soluble salts and alkaline solutions. Especially preferred salts are maleate, fumarate, succinate, S,S tartrate, or R,R tartrate. In addition, suspensions of the active compounds as appropriate oily injection suspensions can be administered. Suitable lipophilic solvents or vehicles can include fatty oils, for example, sesame oil, or synthetic fatty acid esters, for example, ethyl oleate or triglycerides or polyethylene glycol-400 (the compounds are soluble in PEG-400). Aqueous injection suspensions can contain substances that increase the viscosity of the suspension, such as sodium carboxymethyl cellulose, sorbitol, and/or dextran. Optionally, the suspension may also contain stabilizers.

The compounds described herein can be formulated in a nanoparticle delivery system. Such a formulation can be administered systemically (e.g., intravenously) and would cross the blood brain barrier to target cells in the CNS. Examples of nanodelivery-drug complexes include polyethylene glycol (PEG)-coated nanospheres, PEG-treated polyalkylcyanoacrylate nanoparticles, polylactic co-glycolic acid nanoparticles, poly(D,L-lactide)nanoparticles, poly(butylcyanoacrylate) nanoparticles, and polysorbate coated nanoparticles.

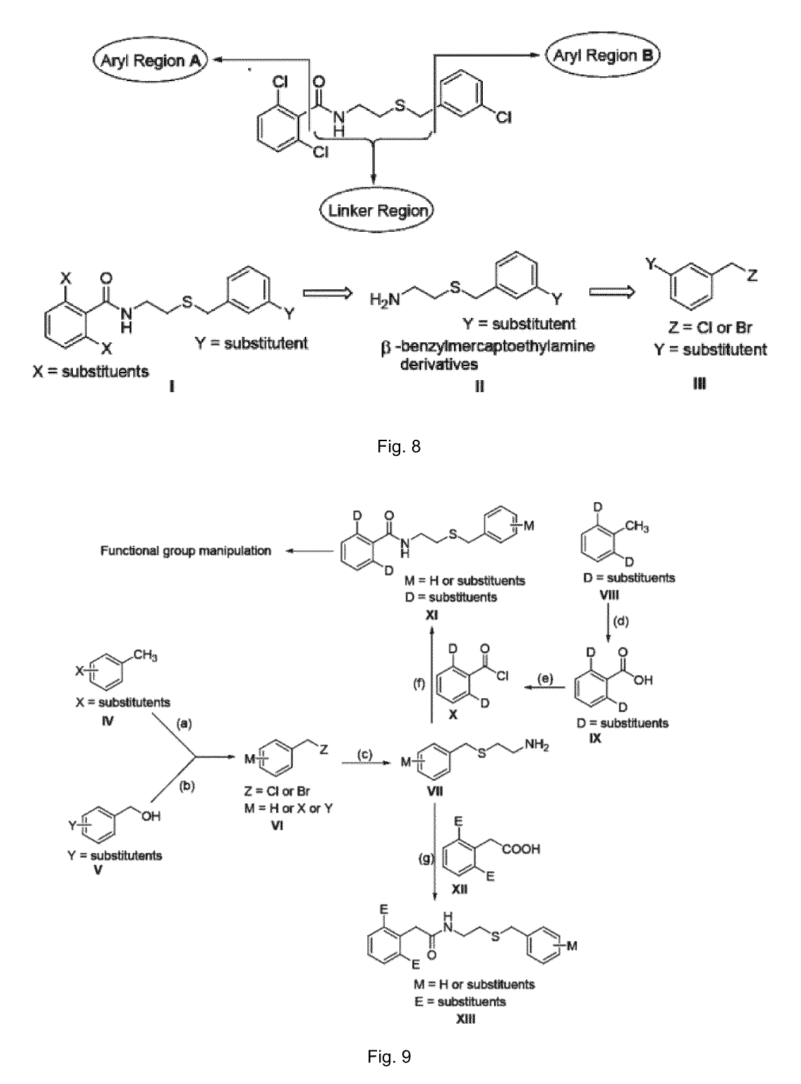

The compounds described herein can be synthesized by iterative modification of three regions of the compound (i.e., the aryl region A, linker region, and aryl region B) starting from commercially available benzyl halides (FIG. 8). As shown in FIG. 8, synthesis can begin with the conversion of the compound to the corresponding β-benzylmercaptoethylamine (β-BMEA) derivatives (compound II). These derivatives can be further be reacted with substituted benzoyl chlorides, giving the desired amides (compound I). By way of example, the compounds can be prepared as shown in FIG. 9 and as discussed in Example 1 below.

The compounds described herein and pharmaceutical compositions including the compounds can be used in a method for inhibiting Aβ secretion in a subject. Due to the key role of α-secretase cleavage activity in the inhibition of Aβ production, the compounds described herein may act to increase α-secretase processing of APP to a neurotrophic and neuroprotective soluble peptide (sAPPα), consequently, decreasing Aβ secretion by precluding Aβ peptide formation in the subject.

In one aspect, the method can include administering to the subject a therapeutically effective amount of a pharmaceutical composition comprising a compound described herein and a pharmaceutical carrier. It will also be appreciated that the pharmaceutical carrier can be selected on the basis of the chosen route of administration and standard pharmaceutical practice. Suitable carriers and their formulation are described, for example, in the book Remington's Pharmaceutical Sciences (Remington's Pharmaceutical Sciences, Mack Publishing Company, Easton, Pa., 1985).

Pharmaceutical compositions of the application can be administered by any means that achieve their intended purpose. For example, administration can be by parenteral, subcutaneous, intravenous, intraarticular, intrathecal, intramuscular, intraperitoneal, or intradermal injections, or by transdermal, enteral, buccal, oromucosal, ocular routes or via inhalation. Alternatively or concurrently, administration can be by the oral route. The dosage administered will be dependent upon the age, health, and weight of the subject, kind of concurrent treatment, if any, frequency of treatment, and the nature of the effect desired. In a particular embodiment, compounds described herein can be administered intravenously to a subject at 100 mg/kg.

The compounds described herein and pharmaceutical compositions including the compounds can be used in the inhibition of Aβ secretion in a subject and treating a neurological or neurodegenerative disorder in a subject. Examples of diseases and disorders that can be treated by the compounds include but are not limited to Alzheimer's disease, Lewy body dementia, AIDS-related dementia, Parkinson's disease, amyotrophic lateral sclerosis, retinitis pigmentosa, spinal muscular atrophy, inclusion body myositis, cerebral amyloid angiopathy, cerebellar degeneration, and nervous system injuries, such as traumatic brain injury, stroke, multiple sclerosis.

Aβ secretion has been implicated in the pathogenesis of several neurodegenerative disorders. The increased expression or processing of APP into Aβ has been reported in a number of rare human mutations and animal models that lead to the production of senile plaques and cognitive impairment. The importance of such observations is substantiated by reports that the presence of high levels of Aβ containing senile plaques has been correlated with the pathogenesis of Alzheimer's disease.

To treat a subject suffering from a neurological disorder, such as Alzheimer's disease, for example, a therapeutically effective amount of a pharmaceutical composition comprising a compound described herein and a pharmaceutical carrier can be administered to the subject via an appropriate route. The pharmaceutical composition can include a compound having the general formulas (I) or (II), and a pharmaceutical carrier. The therapeutically effective amount of the pharmaceutical composition can be administered to the subject via an appropriate route. Upon administration to the subject, the compounds can traverse neuronal cell membranes. In some aspects, the compounds can be formulated in a nanoparticle delivery system in order to aid traversal into neuronal cell membranes. It is contemplated that the compounds can then increase α-secretase activity in the cell to upregulate sAPPα secretion as well as the cellular accumulation of CTF-α that remains in the cell. It is further contemplated that increasing α-secretase activity and sAPPα secretion in a neuronal cell consequently inhibits Aβ peptide secretion, mitigates and/or prevents Aβ aggregation and senile plaque production and promotes neuronal survival.

The following example is for the purpose of illustration only and is not intended to limit the scope of the claims, which are appended hereto.

### Example 1

### Antibodies and Biological Reagents

Mouse anti-Aβ/APP monoclonal antibodies 6E10 (SIG-39320) and 4G8-biotin (SIG-39240), and rabbit anti sAPPβ (SIG-39138) were obtained from Signet (now part of Covance), rabbit polyclonal anti-APP C-terminal antibody (A8717) was obtained from Sigma-Aldrich, rabbit monoclonal anti ADAM-9 (4151) was obtained from Cell Signaling, rabbit polyclonal anti-ADAM-10 (2051) was obtained from ProSci, rabbit polyclonal anti mouse ADAM-17 (AB19026) was obtained from Chemicon, and rabbit anti-GAPDH (ab9485) was obtained from Abcam. Other cell treatment reagents were purchased from Sigma-Aldrich except for the PKC inhibitors (Calbiochem).

### Cell Culture and Drug Treatment

H4βAPP695 wt cells, a human neuroblastoma cell line stably transfected with a wtβAPP695 expression vector construct under the control of a CMV promoter (kindly provided by Dr. Chris Eckman from Mayo Clinic, Florida) were cultured as previously described. Twenty-four hours before drug treatment, the media was replaced with serum-free media. Cells were then incubated with drugs (dissolved in DMSO) or vehicle for 24 h for collection of conditioned media and cell lysates. For the PKC inhibitors experiment, three inhibitors were treated 30 min prior to drug challenge. Primary cortical neurons were prepared from embryonic day 18 APP and presenilin 1 (APPswe, PSEN1dE9) transgenic mice on the C57BL/6 genetic background (Jackson Laboratories, strain no. 5864) as previously described. THLE-3 human hepatoma cells (from ATCC) were plated in 24-well plates that were precoated with 10 μg/mL bovine serum albumin, 10 μg/mL fibronectin, and 30 μg/mL bovine type I collagen. The cells were grown in BEGM media (Clonetics) until confluent and treated with drugs at 20 μM for 24 h. This conditioned media was harvested, diluted 1:1 in serum-free medium, and incubated with H4βAPP695 wt cells for 24 h prior to analysis of secreted Aβ, which was compared to the effect of 10 μM drug treatments without prior incubation with the THLE-3 cells. Caco-2 cells (ATCC) were plated in 12-well plates with Transwell inserts (Costar) at 70000 cells per well and grown for 21 days. The media volume of the apical and basal chambers was 0.5 and 1.5 mL, respectively. Transepithelial resistance was measured in each well and found to be >160 Ω/cm². Drugs at 40 μM and 20 μg/mL of lucifer yellow were added in serum-free medium to the apical chamber. Four hours later, the basal chamber media was harvested and used to measure lucifer yellow fluorescence to confirm monolayer integrity as well as for treatment of H4βAPP695 wt cells for 24 h prior to analysis of secreted Aβ, which was compared to the effect of direct addition of 10 μM of the corresponding drug.

### Assays for Secreted Aβ and Processed APP

Conditioned medium was collected, protease inhibitor cocktail was added, and cell debris was cleared by centrifugation prior to assays for secreted total Aβ by ELISA and sAPPα and sAPPβ by Western blot. The total Aβ ELISA, normalized to total cell lysate protein by BCA assay, was preformed as previously described using the capture antibody 6E10, which recognizes residues 1-16 of human Aβ, a region present in sAPPα but absent in sAPPβ, the long processed forms of secreted APP, and 4G8 for the detection antibody, which is absent in both sAPPα and sAPPβ. For Western blot analysis of sAPPα and sAPPβ, the volume of conditioned media was adjusted by the protein level in the cell lysate prior to SDS gel electrophoresis on 4-12% Bis-Tris SDS gels (Invitrogen). Protein was transferred to PVDF membranes (Invitrogen), blocked with casein blocker (Pierce), and incubated with primary antibodies 6E10, which recognizes sAPPα but not sAPPβ, or with an antibody specific for sAPPβ (both at 1:1000). After washing and incubation with HRP conjugated secondary antibodies, the signals were detected using ECL Western Blotting Detection kit (Pierce).

### Assays for Cell Lysate Proteins

Cells were washed with cold phosphate buffered saline (PBS) and lysed on ice by vigorous mixing over 15 min with RIPA buffer containing 50 mM Tris-HCl (pH 8.0), 150 mM sodium chloride, 1% NP-40, 0.5% sodium deoxycholate, and 0.1% SDS, supplemented with 10% protease inhibitor cocktail and 2 mM PMSF. After centrifugation at 14000 g for 30 min to remove nuclei and other debris at 4° C., the protein content of the supernatants was measured using the BCA assay kit (Pierce). Twenty μg of cell protein was mixed with 4× Nupage loading buffer and with reducing agent (Invitrogen), heated at 70° C. for 10 min, and loaded onto a 4-20% Bis-Tris gel with MES gel running buffer. Blots were prepared and probed with anti APP C-terminus, which binds to both holoAPP, detected at ˜110 KDa, and αCTF detected at ˜10 KDa. An anti GAPDH antibody was also added to detect GAPDH at 38 KDa, which was used as a loading control for normalization. Although this gel system did not routinely detect βCTF, preliminary studies using urea gels demonstrated that the βCTF fragment could be separated from αCTF but is a minor band compared to the αCTF band. For analysis of ADAMS, ADAM10, and ADAM17, the blots were probed with the appropriate antibodies and processed as described above.

### α-Secretase Activity Assay

H4βAPP695 wt were treated with vehicle or 1 for 24 h, washed with ice cold PBS, and lysed in extraction buffer provided with the α-secretase assay kit (R&D Systems) for 10 min on ice and centrifuged at 10000 g for 1 min to remove nuclei. Protein content of the supernatant was determined by the BCA assay, and 50 μg of lysate protein were added to wells of a 96-well assay plate and the volume was adjusted to 100 μL using the lysis buffer. 100 μL of reaction buffer and 10 μL of the fluorogenic substrate were added, and the plates were incubated at 37° C. in the dark with gentle agitation for 2 h. Activity was detected in a fluorescence plate reader using an excitation of 355 nm and an emission of 510 nm after subtraction of fluorescence in the absence of cell lysate.

### In Vivo Drug Administration and Cerebral Aβ Assay

Transgenic mice expressing humanized mutant versions of APP and presenilin 1 (APPswe, PSEN1dE9) on the C57BL/6 genetic background were obtained from Jackson Laboratories (strain no. 5864) and bred to wild type C57BL/6 mice in order to obtain ˜50% transgenic that were used in these studies. These mice develop Aβ senile plaques by 6 months of age. To prepare the drug stock, 17.5 mg of compound 48 was dissolved in 40 μL of DMSO, which was mixed sequentially with 110 μL of Cremophor EL (Sigma) and 900 μL of sterile saline. Two month old female transgenic mice weighing 20-25 g were anesthetized with ketamine/xylazine and injected iv via the retroorbital plexus with vehicle control or with 2.5 mg of compound 48 in 150 μL of the drug stock (yielding a dose of ˜100 to 125 mg/kg). Six h later, the mice were euthanized by CO₂ inhalation, the brains were removed, and the cerebellum discarded. One brain hemisphere was homogenized for 45 s on ice using a tissue grinder in 1 mL of solution containing 5 M guanadine HCl, 50 mM Tris (final pH adjusted to 8.3), and 5% protease inhibitor cocktail. Any debris was removed by centrifugation for 10 min at 2000 g. The supernatant was diluted 1:10 in 20% casein blocker in PBS with 5% protease inhibitor cocktail. The AB1-40 standards were diluted to have an equal concentration of homogenization buffer and other reagents. The protein concentration of the brain homogenate was determined with the BCA assay, and the total Aβ levels were normalized to brain protein levels.

### Statistical Analyses

Western blot band intensities were quantified by densitometric analyses using a BioRad GelDoc 2000 with Quantity One software. Data are presented as the mean±SD. Statistical analyses were carried out using a two-tailed Student's t test using GraphPad Prism software, with p<0.05 regarded as statistically significant.

### Chemistry

Flash chromatography as performed on silica gel (60 Å, 230-400 mesh) purchased from Dynamics Adsorbents (Atlanta, Ga.). Purity of synthesized compounds was assessed using an Agilent 1200 HPLC equipped with an Agilent Eclipse XDB-C18 column (4.6 mm×150 mm) monitoring absorbance at 254 nM. All final products were >95% pure unless otherwise noted in the experimental details. Purities were confirmed by NMR. All purchased compounds tested were at least 95% pure as judged by TLC analysis via densitometry using either anisaldehyde, 2,4-dinitrophenylhydrazine, or iodine as a stain. TLC was done on hard layer, organic binder TLC-plates with a fluorescent indicator and visualized by UV light (254 nm). Solvents and reagents were of commercially available analytical grade quality and used as received without any further purification. ¹H and ¹³C NMR spectra were recorded on a Varian Inova spectrometer (at the Department of Chemistry, CWRU) operating at 400 and 100 MHz for the ¹H and ¹³C NMR spectra, respectively. The internal references were TMS (δ 0.00) and CDCl₃ (δ77.2) for ¹H and ¹³C spectra, respectively. NMR data are presented in the following order: chemical shift, peak multiplicity (s=singlet, d=doublet, t=triplet, q=quartet, m=multiplet, dd=doublet of doublet, bs=broad singlet), coupling constant, proton number. Mass spectra were obtained on a Kratos MS 25 mass spectrometer (at the Department of Chemistry, Case Western Reserve University) using FAB ionization method in m-nitrobenzyl alcohol or glycerol matrices. Melting points were determined with a MEL-TEMP capillary apparatus and are uncorrected. β-Benzylmercaptoethylamine (β-BMEA) derivatives were prepared following our own procedure. 1-Chloromethyl-3-iodo-benzene, 2-bromomethyl-6-chloro-benzonitrile, 2-benzyloxy-ethylamine, and 2,6-dibromobenzoic acid were prepared by following literature procedures. All other chemicals were purchased from commercial sources (Aldrich, Acros, A. K. Scientific, Matrix Scientific, Milestone Pharm Tech, and Wako) and used without any further purification.

### Typical Procedure for the Preparation of β-BMEA Derivatives

LiOH (0.245 g, 10.2 mmol) was dissolved in 5 mL of water, and 15 mL of ethanol was added. The resulting solution was added to a flask containing cysteamine hydrochloride (0.568 g, 5 mmol), followed by the dropwise addition of benzyl halides (5 mmol) with continuous stirring. The reaction mixture was stirred for 40 min at 35° C., and ethanol was removed in vacuo. Subsequently, 20 mL of water was added, and the mixture was extracted with dichloromethane (3×30 mL), dried over anhydrous Na₂SO₄, concentrated in vacuo, and purified via column chromatography over silica gel) using a mobile phase consisting of a suitable mixture of DCM-methanol to afford the chromatographically pure desired β-benzylmercaptoethylamine derivatives.

### Typical Procedure for the Coupling of β-BMEA Derivatives and 2,6-Disubstituted Benzoyl Chlorides

(Synthesis of 2,6-dichloro-N-[2-(3-methoxy-benzylsulfanyl)-ethyl]-benzamide, 27 as representative example). To a solution of 2-(3-methoxy-benzylsulfanyl)-ethylamine (0.394 g, 2 mmol) in dry DCM (15 mL) and Et₃N (0.505 g 5 mmol) was added 2,6-dichloro-benzoyl chloride (0.420 g, 2 mmol) at 0° C. under nitrogen atmosphere. The reaction mixture was further stirred for ˜1 h, during which time it was allowed to warm to room temperature. Solvent evaporation in vacuo gave the crude product, which was purified via column chromatography over silica gel using a mobile phase consisting of a mixture of hexane-ethyl acetate (gradient from 15% v/v ethyl acetate/hexane to 35% v/v ethyl acetate/hexane) to afford the chromatographically pure desired amidated product, 27 in 92% yield (0.68 g), 90% pure by HPLC: mp=60-62° C. ¹H NMR (CDCl₃, 400 MHz): δ 2.65 (t, J=6.4 Hz, 2H), 3.54 (q, J=6.4 Hz, 2H), 3.68 (s, 2H), 3.76 (s, 3H), 6.36 (t, J=5.6 Hz, 1H), 6.72-6.74 (m, 1H), 6.84-6.88 (m, 2H), 7.15-7.25 (m, 4H). ¹³C NMR (CDCl₃, 100 MHz): δ 30.67, 35.82, 38.59, 55.21, 112.78, 114.24, 121.17, 127.98, 129.58, 130.62, 132.13, 135.79, 139.57, 159.71, 164.50. HRMS calculated for C₁₇H₁₈Cl₂NO₂S (M+H)⁺ 370.04353, found 370.04491.

### Typical Procedure for Demethylation of Methoxy Derivatives

(Synthesis of 2,6-dichloro-N-[2-(3-hydroxy-benzylsulfanyl)-ethyl]-benzamide, 29 as representative example). 2,6-Dichloro-N-[2-(3-methoxy-benzylsulfanyl)-ethyl]-benzamide, 27 (370 mg, 1 mmol), was dissolved in anhydrous dichloromethane (10 mL) under a nitrogen atmosphere, and the solution was subsequently cooled to −78° C. A solution of BBr₃ in hexane (2.5 mL of a 1 M solution, 2.5 mmol) was added dropwise. The reaction mixture was stirred for 30 min at −78° C., warmed slowly to room temperature, and stirred an additional 3 h at room temperature. The reaction was quenched by dropwise addition of 1 M HCl (5 mL) at 0° C. followed by the addition of 15 mL of room temperature water, the layers were separated, and the aqueous phase was extracted with EtOAc (3×20 mL). The combined organic layers were washed with water followed by brine, dried over Na₂SO₄, and then concentrated in vacuo. The crude product was purified via column chromatography over silica gel using a mobile phase consisting of a mixture of hexane/ethyl acetate (gradient from 20% v/v ethyl acetate/hexane to 45% v/v ethyl acetate/hexane) as eluent to obtain chromatographically pure desired 29 as off-white solid (0.3 g, 85%): mp=108-110° C. ¹H NMR (CDCl₃, 400 MHz): δ 2.63 (t, J=6.4 Hz, 2H), 3.55 (q, J=6.4 Hz, 2H), 3.61 (s, 2H), 6.43 (t, J=6.0 Hz, 1H), 6.63-6.66 (m, 1H), 6.78-6.8 (m, 2H), 7.07 (t, J=8 Hz, 1H), 7.19-7.29 (m, 3H), 7.31 (bs, 1H). ¹³C NMR (CDCl₃, 100 MHz): δ 30.25, 35.58, 38.97, 114.45, 116.07, 120.64, 128.17, 129.89, 130.99, 132.24, 135.43, 139.52, 156.36, 165.29. HRMS calculated for C₁₆H₁₆Cl₂NO₂S (M+H)⁺ 356.02788, found 356.02903.

### Typical Procedure for the Preparation of Sulfone Derivatives

(Synthesis of 2,6-dichloro-N-[2-(3-methoxy-phenylmethanesulfonyl)-ethyl]-benzamide, 30 as representative example). To a solution of 2,6-dichloro-N-[2-(3-methoxy-benzylsulfanyl)-ethyl]-benzamide, 27 (370 mg, 1 mmol), in glacial acetic acid (6 mL) was added hydrogen peroxide (2 mL, 30% solution). After 24 h stirring at room temperature, the acetic acid was removed under vacuum and the crude sulfone was purified by recrystallization from ethanol (0.3 g, 75%): mp=172-173° C. ¹H NMR (CDCl₃, 400 MHz): δ 3.19-3.22 (m, 2H), 3.83 (s, 3H), 3.91-3.96 (m, 2H), 4.27 (s, 2H), 6.55 (bs, 1H), 6.93-6.99 (m, 3H), 7.23-7.35 (m, 4H). ¹³C NMR (CDCl₃, 100 MHz): δ 32.98, 50.72, 55.59, 60.81, 115.19, 116.35, 123.04, 128.30, 128.93, 130.51, 131.14, 132.39, 135.45, 160.31, 164.95. LRMS calculated for C₁₇H₁₈Cl₂NO₄S (M+H)⁺ 402.0, found 402.1.

### Typical Procedure for the Preparation of Sulfoxide Derivatives

(Synthesis of 2,6-dichloro-N-[2-(3-methoxy-phenylmethanesulfinyl)-ethyl]-benzamide, 32 as representative example). To a solution of 2,6-dichloro-N-[2-(3-methoxy-benzylsulfanyl)-ethyl]-benzamide, 27 (370 mg, 1 mmol), in chloroform at −10° C. was added m-chloroperbenzoic acid (1 mmol) and the resulting mixture was stirred at −10° C. for 12 h. Subsequently, a saturated solution of NaHCO₃ (15 mL) was added, the reaction was stirred for 5 min, followed by extracting with chloroform (2×20 mL). The organic layer was washed with water, dried over Na₂SO₄, concentrated in vacuo, and the crude product was purified via column chromatography over silica gel using a mobile phase consisting of a mixture of hexane/ethyl acetate (gradient from 20% v/v ethyl acetate/hexane to 50% v/v ethyl acetate/hexane) as eluent to obtain chromatographically pure desired product, 32 as white solid (0.247 g, 64%): mp=144-146° C. ¹H NMR (CDCl₃, 400 MHz): δ 2.71-2.76 (m, 1H), 3.03-3.09 (m, 1H), 3.81 (s, 3H), 3.82-3.89 (m, 1H), 3.92 (s, 2H), 3.94-4.01 (m, 1H), 6.77 (t, J=2.0 Hz, 1H), 6.81 (d, J=8.0 Hz, 1H), 6.87-6.89 (m, 1H), 7.21-7.30 (m, 4H), 7.35 (t, J=5.6 Hz, 1H). ¹³C NMR (CDCl₃, 100 MHz): δ 34.56, 49.55, 55.53, 58.46, 114.32, 115.84, 122.51, 128.20, 130.35, 130.49, 130.88, 132.45, 135.96, 160.19, 165.15. HRMS calculated for C₁₇H₁₈Cl₂NO₃S (M+H)⁺ 386.03844, found 386.03650.

### Typical Procedure for Amidation of 2,6-Disubstituted Benzoic Acids Through Acid Chloride Formation

(Synthesis of N-[2-(3-chloro-benzylsulfanyl)-ethyl]-2,6-dimethyl-benzamide, 75 as representative example). One drop of DMF was added to a solution of 2,6-dimethylbenzoic acid (0.3 g, 2 mmol) in thionyl chloride (2 mL), and the mixture was refluxed for 2 h. It was then cooled to room temperature and the excess thionyl chloride was removed in vacuo. The solid residue was dissolved in dry DCM (5 mL) and cooled to 0° C. A mixture of β-(3-chlorobenzyl)mercaptoethylamine (0.404 g, 2 mmol) and triethylamine (1 mL, 7 mmol) in dry DCM (5 mL) was added in dropwise manner at 0° C. under nitrogen atmosphere, and the mixture was allowed to stir at room temperature for 1.5 h. After the completion of reaction (as judged by TLC) the solvent was removed in vacuo and the solid residue was dissolved in DCM (30 mL). The organic phase was washed with brine (15 mL) and water (15 mL), dried over Na₂SO₄, and concentrated in vacuo. The crude residue was purified by silica gel column chromatography using a mixture of hexane/ethyl acetate (gradient from 15% v/v ethyl acetate/hexane to 35% v/v ethyl acetate/hexane) as eluent to obtain the desired amidated product, 75 (0.507 g, 76%): mp=53-55° C. ¹H NMR (CDCl₃, 400 MHz): δ 2.27 (s, 6H), 2.65 (t, J=6.4 Hz, 2H), 3.57 (q, J=6.4 Hz, 2H), 3.69 (s, 2H), 6.06 (bs, 1H), 6.99 (d, J=7.6 Hz, 2H), 7.14 (t, J=7.6 Hz, 1H), 7.19-7.23 (m, 3H), 7.32 (s, 1H). ¹³C NMR (CDCl₃, 100 MHz): δ 19.41, 31.42, 35.46, 38.11, 127.27, 127.65, 127.71, 128.96, 129.12, 130.07, 134.39, 134.65, 137.61, 140.32, 170.61. HRMS calculated for C₁₈H₂₁ClNOS (M+H)⁺ 334.10324, found 334.10221.

### Procedure for DCC-Mediated Coupling of Carboxylic Acids

(Synthesis of N-[2-(3-chloro-benzylsulfanyl)-ethyl]-2-(2,6-dichloro-phenyl)-acetamide, 78 as representative example) To a solution of 2,6-dichlorophenylacetic acid (0.41 g, 2 mmol) in dry DCM (10 mL) was added DCC (0.435 g, 2.1 mmol) and resultant mixture was stirred under N₂ for 30 min. A mixture of β-(3-chlorobenzyl)mercaptoethylamine (0.404 g, 2 mmol) and triethylamine (0.252 g, 2.5 mmol) in dry DCM (4 mL) was added and the resultant mixture was left for stirring at room temperature under N₂ for 1 h. The reaction mixture was then filtered to remove the precipitated dicyclohexylurea (DHU). The filtrate was concentrated in vacuo and the crude product was dissolved with cold EtOAc to effect the precipitation of more DHU, which was removed by further filtration. The organic layer was concentrated in vacuo and the crude product was purified by silica gel column chromatography using a mixture of hexane/ethyl acetate (gradient from 15% v/v ethyl acetate/hexane to 35% v/v ethyl acetate/hexane) as eluent to obtain the desired amidated product, 78 (0.62 g, 80%): mp=116-118° C. ¹H NMR (CDCl₃, 400 MHz): δ 2.54 (bs, 2H), 3.38-3.39 (m, 2H), 3.63 (s, 2H), 3.92 (s, 2H), 5.88 (bs, 1H), 7.16-7.36 (m, 7H). ¹³C NMR (CDCl₃, 100 MHz): δ 31.35, 35.49, 38.39, 39.06, 127.24, 127.59, 128.64, 129.09, 129.44, 130.04, 131.78, 134.59, 136.52, 140.36, 168.53. HRMS calculated for C₁₇H₁₇Cl₃NOS (M+H)⁺ 388.00964, found 388.00983.

Specific Procedures for Reactions Deviating from Typical Conditions

2,6-Dichloro-N-(2-[3-(N-hydroxycarbamimidoyl)-benzylsulfanyl]-ethyl)-benzamide (39) 2,6-Dichloro-N-[2-(3-cyano-benzylsulfanyl)-ethyl]-benzamide, 38 (0.365 g, 1 mmol), hydroxylamine hydrochloride (0.278 g, 4 mmol) and K₂CO₃ (0.276 g, 2 mmol) were dissolved in a mixture of water (6 mL) and ethanol (9 mL). The solution was gently stirred for 10 min and then was heated under reflux for 5 h. The resulting solution was cooled, concentrated in vacuo, and the residue was partitioned between water and DCM. The organic extracts were dried over Na₂SO₄, evaporated in vacuo, and the crude product was purified via column chromatography over silica gel using a mobile phase consisting of a mixture of hexane/ethyl acetate (gradient from 25% v/v ethyl acetate/hexane to 80% v/v ethyl acetate/hexane) as eluent to obtain chromatographically pure desired, 39 (0.165 g, 42%) as a white solid: mp=126-127° C. ¹H NMR (DMSO-d₆, 400 MHz): δ 2.54 (t, J=6.8 Hz, 2H), 3.39 (q, J=6.4 Hz, 2H), 3.78 (s, 2H), 5.75 (bs, 2H), 7.27-7.52 (m, 6H), 7.63 (s, 1H), 8.82 (t, J=5.2 Hz, 1H), 9.59 (s, 1H). ¹³C NMR (DMSO-d₆, 100 MHz): δ 30.38, 35.39, 39.36, 124.62, 126.60, 128.74, 128.81, 130.10, 131.65, 131.77, 134.16, 137.07, 139.06, 151.32, 164.24. HRMS calculated for C₁₇H₁₈Cl₂N₃O₂S (M+H)⁺ 398.04968, found 398.04691.

### N-[2-(3-Amino-benzylsulfanyl)-ethyl]-2,6-dichloro-benzamide (41)

2,6-Dichloro-N-[2-(3-nitro-benzylsulfanyl)-ethyl]-benzamide, 40 (0.385 g, 1 mmol), and sodium acetate (0.41 g, 5 mmol) were added to 15 mL of EtOH, and the mixture was heated to 75° C. until the solid dissolved completely. SnCl₂.2H₂O (1.12 g, 5 mmol) was added, and the reaction mixture was subsequently stirred at 75° C. for 40 min. After removal of solvent in vacuo, the residue was dissolved in 40 mL EtOAc and washed with 45% K₂CO₃ (25 mL), dried over anhydrous Na₂SO₄, concentrated in vacuo, and purified via column chromatography over silica gel using a mobile phase consisting of a mixture of hexane/ethyl acetate (gradient from 25% v/v ethyl acetate/hexane to 70% v/v ethyl acetate/hexane) as eluent to obtain chromatographically pure 41 (0.265 g, 75%): mp=55-56° C. ¹H NMR (CDCl₃, 400 MHz): δ 2.67 (t, J=6.4 Hz, 2H), 3.57 (q, J=6.4 Hz, 2H), 3.62 (s, 2H), 3.69 (bs, 2H), 6.29 (bs, 1H), 6.49-6.52 (m, 1H), 6.64-6.68 (m, 2H), 7.03 (t, J=7.6 Hz, 1H), 7.21-7.31 (m, 3H). ¹³C NMR (CDCl₃, 100 MHz): δ 30.82, 35.99, 38.83, 114.22, 115.67, 119.18, 128.25, 129.74, 130.87, 132.42, 136.09, 139.27, 146.97, 164.73. LRMS calculated for C₁₆H₁₇Cl₂N₂OS (M+H)⁺ 355.0, found 355.2.

### 3-[2-(2,6-Dichloro-benzoylamino)-ethylsulfanylmethyl]-benzoic Acid (44)

To a solution of 3-[2-(2,6-dichloro-benzoylamino)-ethylsulfanylmethyl]-benzoic acid methyl ester, 43 (0.398 g, 1 mmol), in THF (7 mL) was added 5 mL of aqueous LiOH (0.072 g, 3 mmol) solution and the resulting mixture was stirred at 60° C. (˜2 h) until the ester starting material was consumed completely (as judged by TLC). The mixture was then allowed to cool to room temperature, and solvent was removed in vacuo followed by dilution of the residue with water. The resulting aqueous mixture was extracted once with EtOAc. The pH of the aqueous phase was adjusted to approximately 1 via the addition of 1 M HCl. The precipitate was filtered off, washed with water, and dried, yielding 44 (0.29 g, 75%) as a white solid, 90% pure by HPLC: mp=165-166° C. ¹H NMR (acetone-d₆, 400 MHz): δ 2.69-2.73 (m, 2H), 3.57-3.62 (m, 2H), 3.92 (s, 2H), 7.37-7.47 (m, 4H), 7.65-7.67 (m, 1H), 7.89-7.92 (m, 2H), 8.05 (t, J=2.0 Hz, 1H). ¹³C NMR (acetone-d₆, 100 MHz): δ 30.48, 35.09, 39.17, 128.21, 128.37, 128.79, 130.32, 130.95, 131.06, 132.00, 133.79, 136.97, 139.69, 164.05, 166.87. HRMS calculated for C₁₇H₁₆Cl₂NO₃S (M+H)⁺ 384.02279, found 384.02352.

### N-[2-(3-Carbamoyl-benzylsulfanyl)-ethyl]-2,6-dichloro-benzamide (45)

To a stirred solution of 2,6-dichloro-N-[2-(3-cyano-benzylsulfanyl)-ethyl]-benzamide, 38 (0.365 g, 1 mmol), in DMSO (1.5 mL) at 0° C. was added 30% H₂O₂ (0.12 mL) and anhydrous K₂CO₃ (0.03 g). The reaction mixture was allowed to warm to room temperature and stirred for an additional 10 min. Then 30 mL of water was added to the reaction mixture followed by stifling for an additional 10 min. The aqueous phase was extracted with EtOAc (3×20 mL), dried over anhydrous Na₂SO₄, concentrated in vacuo, and the crude product was purified via column chromatography over silica gel using a mobile phase consisting of a mixture of hexane/ethyl acetate (gradient from 25% v/v ethyl acetate/hexane to 75% v/v ethyl acetate/hexane) as eluent to obtain chromatographically pure 45 (0.25 g, 65%) as a white solid: mp=148-150° C. ¹H NMR (CD₃OD, 400 MHz): δ 2.61-2.65 (m, 2H), 3.50-3.53 (m, 2H), 3.84 (s, 2H), 7.32-7.43 (m, 4H), 7.55-7.57 (m, 1H), 7.73-7.75 (m, 1H), 7.86-7.87 (m, 1H). ¹³C NMR (CD₃OD, 100 MHz): δ 29.59, 34.94, 39.06, 126.23, 128.02, 128.12, 128.61, 130.95, 132.01, 132.47, 133.98, 136.08, 139.39, 165.98, 171.03. HRMS calculated for C₁₇H₁₇Cl₂N₂O₂S (M+H)⁺ 383.03878, found 383.03804.

### 2,6-Dichloro-N-[2-(3-formyl-benzylsulfanyl)-ethyl]-benzamide (49)

To a stirred solution of 2,6-dichloro-N-[2-(3-cyano-benzylsulfanyl)-ethyl]-benzamide, 38 (0.365 g, 1 mmol), in dry THF (10 mL) at −78° C. under nitrogen atmosphere was added 1 M solution of DIBAL-H in toluene (1.2 mL, 1.2 mmol) dropwise. The reaction mixture was stirred and gradually warmed from −78° C. to room temperature over 2 h. The reaction mixture was then quenched by addition of a mixture of saturated aqueous NH₄Cl solution and 6 M aqueous HCl (5:1, v/v) at 0° C. The organic solvent was removed in vacuo, and the residue was partitioned between ethyl acetate and brine. The organic solvent was dried over anhydrous Na₂SO₄, concentrated in vacuo, and the crude product was purified via column chromatography over silica gel using a mobile phase consisting of a mixture of hexane/ethyl acetate (gradient from 20% v/v ethyl acetate/hexane to 45% v/v ethyl acetate/hexane) as eluent to obtain chromatographically pure 49 as white solid (0.19 g, 52%): mp=63-65° C. ¹H NMR (CDCl₃, 400 MHz): δ 2.71 (t, J=6.4 Hz, 2H), 3.63 (q, J=6.4 Hz, 2H), 3.83 (s, 2H), 6.28 (bs, 1H), 7.23-7.31 (m, 3H), 7.49 (t, J=7.6 Hz, 1H), 7.63 (d, J=7.6 Hz, 1H), 7.76 (d, J=7.6 Hz, 1H), 7.85 (s, 1H), 9.99 (s, 1H). ¹³C NMR (CDCl₃, 100 MHz): δ 31.03, 35.56, 38.76, 128.29, 128.86, 129.61, 130.19, 130.96, 132.39, 135.19, 135.94, 136.92, 139.51, 164.78, 192.38. HRMS calculated for C₁₇H₁₆Cl₂NO₂S (M+H)⁺ 368.02788, found 368.02880.

### [2-(3-Chloro-benzylsulfanyl)-ethyl]-(2,6-dichloro-benzyl)-amine (27)

To a stirred solution of β-(3-chlorobenzyl)mercaptoethylamine (0.202 g, 1 mmol) and K₂CO₃ (0.276 g, 2 mmol) in 5 mL of dry DMF was added 2,6-dichlorobenzyl bromide (0.24 g, 1 mmol) in a dropwise manner. The resulting mixture was allowed to stir for 18 h at room temperature. The reaction mixture was poured into 35 mL of water and stirred for 10 min. The aqueous layer was extracted with DCM (2×25 mL), the combined organic extracts was dried over anhydrous Na₂SO₄, concentrated in vacuo, and purified via column chromatography over silica gel using a mobile phase consisting of a mixture of hexane/ethyl acetate (gradient from 25% v/v ethyl acetate/hexane to 80% v/v ethyl acetate/hexane) to yield chromatographically pure 57 as colorless liquid (0.216 g, 60%). ¹H NMR (CDCl₃, 400 MHz): δ 1.96 (s, 1H), 2.59 (t, J=6.4 Hz, 2H), 2.77 (t, J=6.4 Hz, 2H), 3.60 (s, 2H), 4.08 (s, 2H), 7.12-7.22 (m, 4H), 7.27 (s, 1H), 7.31 (d, J=8.0 Hz, 2H). ¹³C NMR (CDCl₃, 100 MHz): δ 31.95, 35.63, 47.09, 48.18, 127.19, 127.44, 128.63, 129.12, 129.19, 129.94, 134.50, 135.81, 136.16, 140.63. HRMS calculated for C₁₆H₁₇Cl₃NS (M+H)⁺ 360.01473, found 360.01422.

### 2,6-Dichloro-N-(2-[3-(cyclopropanecarbonyl-amino)-benzylsulfanyl]-ethyl)-benzamide (69)

To a solution of N-[2-(3-amino-benzylsulfanyl)-ethyl]-2,6-dichloro-benzamide, 41 (0.423 g, 1 mmol), and Et₃N (0.303 g, 3 mmol) in dry CH₂Cl₂ (10.0 mL) was added cyclopropanecarbonyl chloride (0.105 g, 1 mmol) at room temperature. The resulting solution was refluxed for 12 h, cooled to room temperature, and the solvent was removed in vacuo to afford a thick oily residue which was purified by column chromatography over silica gel using a mobile phase consisting of a mixture of hexane/ethyl acetate (gradient from 25% v/v ethyl acetate/hexane to 50% v/v ethyl acetate/hexane) as eluent to obtain chromatographically pure 69 (0.21 g, 50%) as a white solid: mp=46-47° C. ¹H NMR (CDCl₃, 400 MHz): δ 0.63-0.65 (m, 2H), 0.74 (bs, 2H), 1.47-1.49 (m, 1H), 2.61 (t, J=6.8 Hz, 2H), 3.44 (q, J=6.8 Hz, 2H), 3.64 (s, 2H), 6.98 (d, J=7.6 Hz, 1H), 7.16-7.27 (m, 6H), 7.57 (s, 1H), 8.57 (s, 1H). ¹³C NMR (CDCl₃, 100 MHz): δ 8.05, 15.46, 30.32, 35.83, 38.97, 118.88, 121.24, 124.42, 128.15, 129.47, 130.76, 132.33, 136.09, 138.49, 138.79, 165.16, 172.95. HRMS calculated for C₂₀H₂₁Cl₂N₂O₂S (M+H)⁺ 423.07008, found 423.07069.

### 2,6-Dichloro-N-(2-(3-[2-(2-hydroxy-ethoxy)-ethoxy]-benzylsulfanyl)-ethyl)-benzamide (70)

To a stirred solution of 2,6-dichloro-N-[2-(3-hydroxy-benzylsulfanyl)-ethyl]-benzamide, 29 (0.356 g, 1 mmol) and K₂CO₃ (0.414 g, 3 mmol) in 4 mL of dry DMF was added ethylene glycol mono-2-chloroethyl ether (0.13 g, 1.05 mmol). The resulting mixture was allowed to stir for 12 h at 100° C. After cooling to room temperature, water was added (25 mL) and the reaction mixture was extracted with DCM (2×25 mL). The combined organic extracts were dried over anhydrous Na₂SO₄, concentrated in vacuo, and purified via column chromatography over silica gel using a mobile phase consisting of a mixture of hexane/ethyl acetate (gradient from 25% v/v ethyl acetate/hexane to 80% v/v ethyl acetate/hexane) to yield chromatographically pure 70 as colorless liquid (0.230 g, 52%), 86% pure by HPLC. ¹H NMR (CDCl₃, 400 MHz): δ 2.68 (t, J=6.4 Hz, 2H), 3.54-3.63 (m, 4H), 3.66-3.70 (m, 4H), 3.82-3.84 (m, 2H), 4.11-4.13 (m, 2H), 6.76-6.83 (m, 2H), 6.90-6.92 (m, 2H), 7.17-7.29 (m, 4H). ¹³C NMR (CDCl₃, 100 MHz): δ 30.73, 35.97, 38.90, 61.77, 67.55, 69.81, 72.77, 113.59, 115.26, 121.73, 128.14, 129.81, 130.76, 132.34, 136.17, 139.88, 159.02, 164.79. HRMS calculated for C₂₀H₂₄Cl₂NO₄S (M+H)⁺ 444.08031, found 444.07924.

### 2,6-Dichloro-N-[2-(4-methoxy-benzylsulfanyl)-ethyl]-benzamide (26)

The compound was prepared using 2-(4-methoxy-benzylsulfanyl)-ethylamine as the amine component and following the procedure described for 27: mp=80-81° C. ¹H NMR (CDCl₃, 400 MHz): δ 2.68 (t, J=6.4 Hz, 2H), 3.60 (q, J=6.0 Hz, 2H), 3.69 (s, 3H), 3.78 (s, 3H), 6.13 (bs, 1H), 6.80-6.83 (m, 2H), 7.22-7.31 (m, 5H). ¹³C NMR (CDCl₃, 100 MHz): δ 30.75, 35.27, 38.56, 55.38, 114.13, 128.15, 129.91, 130.05, 130.77, 132.33, 135.96, 158.84, 164.59. HRMS calculated for C₁₇H₁₈Cl₂NO₂S (M+H)⁺ 370.04353, found 370.04217.

### 2,6-Dichloro-N-[2-(4-hydroxy-benzylsulfanyl)-ethyl]-benzamide (28)

The compound was prepared from 26 using the procedure described for 29: mp=138-140° C. ¹H NMR (acetone-d₆, 400 MHz): δ 2.66-2.69 (m, 2H), 3.54-3.59 (m, 2H), 3.74 (s, 2H), 6.76-6.79 (m, 2H), 7.19-7.23 (m, 2H), 7.36-7.43 (m, 3H), 7.85 (bs, 1H), 8.32 (s, 1H). ¹³C NMR (acetone-d₆, 100 MHz): δ 30.15, 34.93, 39.26, 115.39, 128.22, 129.43, 130.36, 130.95, 132.01, 137.01, 156.64, 164.03. HRMS calculated for C₁₆H₁₆Cl₂NO₂S (M+H)⁺ 356.02788, found 356.02820.

### 2,6-Dichloro-N-[2-(3-hydroxy-phenylmethanesulfonyl)-ethyl]-benzamide (31)

The compound was prepared from 30 using the procedure described for 29: mp=139-141° C. ¹H NMR (acetone-d₆, 400 MHz): δ 3.34-3.38 (m, 2H), 3.82-3.87 (m, 2H), 4.43 (s, 2H), 6.85-6.88 (m, 1H), 6.99 (d, J=7.6 Hz, 2H), 7.22 (t, J=7.6 Hz, 1H), 7.41-7.43 (m, 3H), 7.98 (bs, 1H), 8.56 (s, 1H). ¹³C NMR (acetone-d₆, 100 MHz): δ 33.38, 50.63, 59.29, 115.87, 118.21, 122.40, 128.28, 129.88, 130.21, 131.25, 131.98, 136.45, 157.76, 164.46. LRMS calculated for C₁₆H₁₆Cl₂NO₄S (M+H)⁺ 388.0, found 388.2.

### 2,6-Dichloro-N-[2-(3-hydroxy-phenylmethanesulfinyl)-ethyl]-benzamide (33)

The compound was prepared from 29 using the procedure described for 32: mp=165-166° C. ¹H NMR (CD₃OD, 400 MHz): δ 2.91-2.95 (m, 1H), 3.13-3.20 (m, 1H), 3.68-3.75 (m, 1H), 3.81-3.85 (m, 1H), 4.06-4.16 (m, 2H), 6.76-6.84 (m, 3H), 7.19 (t, J=8.0 Hz, 1H), 7.37-7.42 (m, 3H). ¹³C NMR (DMSO-d₆, 100 MHz): δ 33.77, 50.96, 58.53, 115.70, 117.42, 121.16, 128.14, 129.99, 130.84, 131.73, 132.17, 136.66, 158.00, 164.89. HRMS calculated for C₁₆H₁₆Cl₂NO₃S (M+H)⁺ 372.02279, found 372.02187.

### 2,6-Dimethoxy-N-[2-(3-methoxy-benzylsulfanyl)-ethyl]-benzamide (34)

The compound was prepared using 2,6-dimethoxybenzoyl chloride as acid chloride and following the procedure described for 27: mp=41-43° C. ¹H NMR (CDCl₃, 400 MHz): δ 2.69 (t, J=6.4 Hz, 2H), 3.60 (q, J=6.4 Hz, 2H), 3.73 (s, 2H), 3.79 (s, 9H), 6.13 (bs, 1H), 6.55 (d, J=8.4 Hz, 2H), 6.78 (dd, J=1.6 Hz, J=7.6 Hz, 1H), 6.89-6.92 (m, 2H), 7.19-7.29 (m, 2H). ¹³C NMR (CDCl₃, 100 MHz): δ 31.33, 35.99, 38.78, 55.45, 56.18, 104.22, 113.01, 114.49, 115.89, 121.46, 129.79, 130.96, 139.97, 157.66, 159.99, 166.17. HRMS calculated for C₁₉H₂₄NO₄S (M+H)⁺ 362.14260, found 362.14429.

### 2,6-Dihydroxy-N-[2-(3-hydroxy-benzylsulfanyl)-ethyl]-benzamide (35)

The compound was prepared from 34 using the procedure described for 29: mp=61-62° C. ¹H NMR (acetone-d₆, 400 MHz): δ 2.77 (t, J=6.4 Hz, 2H), 3.84 (q, J=6.4 Hz, 2H), 4.11 (s, 2H), 6.68-6.75 (m, 3H), 6.82 (d, J=8.8 Hz, 1H), 6.93 (s, 1H), 7.02 (t, J=8.0 Hz, 1H), 7.67 (t, J=8.4 Hz, 1H), 10.49 (bs, 1H). ¹³C NMR (acetone-d₆, 100 MHz): δ 35.39, 41.24, 57.24, 103.83, 112.16, 114.30, 116.14, 119.87, 129.47, 139.11, 139.71, 157.87, 159.86, 165.52. LRMS calculated for C₁₆H₁₈NO₄S (M+Na)⁺ 342.1, found 342.2.

### N-[2-(3-Chloro-benzylsulfanyl)-ethyl]-2,6-dimethoxy-benzamide (36)

The compound was prepared following the procedure described for 27. 2,6-Dimethoxybenzoyl chloride was used as acid chloride and 2-(3-chloro-benzylsulfanyl)-ethylamine as the amine component: mp=90-91° C. ¹H NMR (CDCl₃, 400 MHz): δ 2.68 (t, J=6.4 Hz, 2H), 3.60 (q, J=6.4 Hz, 2H), 3.72 (s, 2H), 3.79 (s, 6H), 6.13 (bs, 1H), 6.55 (d, J=8.4 Hz, 2H), 7.19-7.29 (m, 4H), 7.33 (s, 1H). ¹³C NMR (CDCl₃, 100 MHz): δ 31.38, 35.51, 38.81, 56.19, 104.23, 115.81, 127.34, 127.55, 129.15, 130.05, 130.99, 134.59, 140.55, 157.65, 166.18. HRMS calculated for C₁₈H₂₁ClNO₃S (M+H)⁺ 366.09307, found 366.09277.

### N-[2-(3-Methoxy-benzylsulfanyl)-ethyl]-2,6-dinitro-benzamide (37)

The compound was prepared using 2,6-dinitrobenzoyl chloride as acid chloride and following the procedure described for 27: mp=69-71° C. ¹H NMR (CDCl₃, 400 MHz): δ 2.71 (t, J=6.4 Hz, 2H), 3.54 (q, J=6.0 Hz, 2H), 3.69 (s, 2H), 3.77 (s, 3H), 6.45 (bs, 1H), 6.71 (d, J=7.2 Hz, 1H), 6.85-6.89 (m, 2H), 7.17 (t, J=7.6 Hz, 1H), 7.73 (t, J=8.4 Hz, 1H), 8.33 (d, J=8.0 Hz, 2H). ¹³C NMR (CDCl₃, 100 MHz): δ 30.61, 36.07, 39.15, 55.44, 113.09, 114.39, 121.39, 128.01, 129.68, 129.81, 130.91, 139.79, 147.53, 159.96, 161.90. LRMS calculated for C₁₇H₁₈N₃O₆S (M+H)⁺ 392.1, found 392.3.

### 2,6-Dichloro-N-[2-(3-cyano-benzylsulfanyl)-ethyl]-benzamide (38)

The compound was prepared using 3-(2-amino-ethylsulfanylmethyl)-benzonitrile as the amine component and following the procedure described for 27: mp=123-125° C. ¹H NMR (CDCl₃, 400 MHz): δ 2.69 (t, J=6.4 Hz, 2H), 3.63 (q, J=6.4 Hz, 2H), 3.78 (s, 2H), 6.26 (bs, 1H), 7.24-7.32 (m, 3H), 7.43 (t, J=7.6 Hz, 1H), 7.54 (d, J=7.6 Hz, 1H), 7.60 (d, J=7.6 Hz, 1H), 7.65 (s, 1H). ¹³C NMR (CDCl₃, 100 MHz): δ 31.05, 35.33, 38.74, 112.94, 118.81, 128.31, 129.67, 131.00, 131.15, 132.39, 132.55, 133.67, 135.89, 139.91, 164.82. HRMS calculated for C₁₇H₁₅Cl₂N₂OS (M+H)⁺ 365.02821, found 365.02794.

### 2,6-Dichloro-N-[2-(3-nitro-benzylsulfanyl)-ethyl]-benzamide (40)

The compound was prepared using 2-(3-nitro-benzylsulfanyl)-ethylamine as the amine component and following the procedure described for 27: mp=76-78° C. ¹H NMR (CDCl₃, 400 MHz): δ 2.72 (t, J=6.4 Hz, 2H), 3.64 (q, J=6.4 Hz, 2H), 3.86 (s, 2H), 6.27 (bs, 1H), 7.23-7.32 (m, 3H), 7.50 (t, J=7.6 Hz, 1H), 7.71 (d, J=7.6 Hz, 1H), 8.11 (d, J=8.0 Hz, 1H), 8.22 (s, 1H). ¹³C NMR (CDCl₃, 100 MHz): δ 31.15, 35.45, 38.79, 122.53, 123.92, 128.30, 129.80, 130.99, 132.38, 135.29, 135.87, 140.51, 148.63, 164.80. HRMS calculated for C₁₆H₁₅Cl₂N₂O₃S (M+H)⁺ 385.01804, found 385.02043.

### 2-Chloro-6-methoxy-N-[2-(3-methoxy-benzylsulfanyl)-ethyl]-benzamide (42)

The compound was prepared using 2-chloro-6-methoxy-benzoyl chloride as the acid chloride and following the procedure described for 27: mp=58-59° C. ¹H NMR (CDCl₃, 400 MHz): δ 2.68 (t, J=6.4 Hz, 2H), 3.59 (q, J=6.4 Hz, 2H), 3.71 (s, 2H), 3.78 (s, 3H), 3.79 (s, 3H), 6.17 (s, 1H), 6.75-6.81 (m, 2H), 6.88-6.91 (m, 2H), 6.97 (d, J=8.0 Hz, 1H), 7.18-7.27 (m, 2H). ¹³C NMR (CDCl₃, 100 MHz): δ 31.13, 36.00, 38.74, 55.74, 56.30, 109.67, 113.02, 114.86, 121.41, 121.86, 126.38, 129.80, 130.89, 132.11, 139.85, 157.43, 159.97, 165.37. HRMS calculated for C₁₈H₂₁ClNO₃S (M+H)⁺ 366.09307, found 366.09276.

### 3-[2-(2,6-Dichloro-benzoylamino)-ethylsulfanylmethyl]-benzoic Acid Ethyl Ester (43)

The compound was prepared using 3-(2-amino-ethylsulfanylmethyl)-benzoic acid ethyl ester as the amine component and following the procedure described for 27: mp=43-45° C. ¹H NMR (CDCl₃, 400 MHz): δ 1.39 (t, J=7.2 Hz, 3H), 2.71 (t, J=6.4 Hz, 2H), 3.62 (q, J=6.4 Hz, 2H), 3.79 (s, 2H), 4.37 (q, J=7.2 Hz, 2H) 6.22 (bs, 1H), 7.22-7.31 (m, 3H), 7.38 (t, J=7.6 Hz, 1H), 7.54 (d, J=7.6 Hz, 1H), 7.91 (d, J=8.0 Hz, 1H), 7.98 (s, 1H). ¹³C NMR (CDCl₃, 100 MHz): δ 14.55, 31.14, 35.77, 38.69, 61.33, 128.26, 128.67, 128.93, 130.04, 130.90, 131.09, 132.43, 133.50, 135.99, 138.61, 164.73, 166.56. HRMS calculated for C₁₉H₂₀Cl₂NO₃S (M+H)⁺ 412.05409, found 412.05431.

### 2,6-Difluoro-N-[2-(3-fluoro-benzylsulfanyl)-ethyl]-benzamide (46)

The compound was prepared following the procedure described for 27. 2,6-Difluorobenzoyl chloride was used as acid chloride and 2-(3-fluoro-benzylsulfanyl)-ethylamine as the amine component, 92% pure by HPLC: mp=39-41° C. ¹H NMR (CDCl₃, 400 MHz): δ 2.68 (t, J=6.4 Hz, 2H), 3.59 (q, J=6.4 Hz, 2H), 3.74 (s, 2H), 6.39 (bs, 1H), 6.92-6.96 (m, 3H), 7.06-7.11 (m, 2H), 7.24-7.29 (m, 1H), 7.33-7.39 (m, 1H). ¹³C NMR (CDCl₃, 100 MHz): δ 31.09, 35.64, 38.77, 112.12, 112.17, 112.38, 114.33, 114.54, 115.79, 116.02, 124.73, 124.76, 130.24, 130.33, 131.88, 131.98, 132.08, 140.74, 140.82, 158.91, 158.97, 160.67, 161.42, 161.48, 161.89, 164.35. HRMS calculated for C₁₆H₁₅F₃NOS (M+H)⁺ 326.08264, found 326.08419.

### 2,6-Dichloro-N-[2-(3-fluoro-benzylsulfanyl)-ethyl]-benzamide (47)

The compound was prepared using 2-(3-fluoro-benzylsulfanyl)-ethylamine as the amine component and following the procedure described for 27: mp=70-72° C. ¹H NMR (CDCl₃, 400 MHz): δ 2.70 (t, J=6.4 Hz, 2H), 3.61 (q, J=6.4 Hz, 2H), 3.74 (s, 2H), 6.18 (bs, 1H), 6.91-6.96 (m, 1H), 7.04-7.11 (m, 2H), 7.23-7.32 (m, 4H). ¹³C NMR (CDCl₃, 100 MHz): δ 31.05, 35.61, 35.62, 38.67, 114.35, 114.57, 115.82, 116.03, 124.74, 124.77, 128.28, 130.27, 130.35, 130.92, 132.43, 135.98, 140.71, 140.79, 161.89, 164.34, 164.74. HRMS calculated for C₁₆H₁₅Cl₂FNOS (M+H)⁺ 358.02354, found 358.02334.

### 2,6-Dichloro-N-[2-(3-methyl-benzylsulfanyl)-ethyl]-benzamide (48)

The compound was prepared using 2-(3-methyl-benzylsulfanyl)-ethylamine as the amine component and following the procedure described for 27: mp=56-57° C. ¹H NMR (CDCl₃, 400 MHz): δ 2.32 (s, 3H), 2.69 (t, J=6.4 Hz, 2H), 3.59 (t, J=6.4 Hz, 2H), 3.70 (s, 2H), 6.15 (bs, 1H), 7.02 (d, J=7.2 Hz, 1H), 7.09-7.19 (m, 3H), 7.24-7.33 (m, 3H). ¹³C NMR (CDCl₃, 100 MHz): δ 21.59, 31.02, 36.00, 38.70, 126.09, 128.22, 128.26, 128.74, 129.79, 130.87, 132.47, 136.09, 138.05, 138.59, 164.69. HRMS calculated for C₁₇H₁₈Cl₂NOS (M+H)⁺ 354.04861, found 354.04895.

### 2,6-Dichloro-N-[2-(3-trifluoromethyl-benzylsulfanyl)-ethyl]-benzamide (50)

The compound was prepared using 2-(3-trifluoromethyl-benzylsulfanyl)-ethylamine as the amine component and following the procedure described for 27: mp=54-55° C. ¹H NMR (CDCl₃, 400 MHz): δ 2.70 (t, J=6.4 Hz, 2H), 3.62 (q, J=6.4 Hz, 2H), 3.80 (s, 2H), 6.23 (bs, 1H), 7.23-7.31 (m, 3H), 7.43 (t, J=7.6 Hz, 1H), 7.49-7.54 (m, 2H), 7.59 (s, 1H). ¹³C NMR (CDCl₃, 100 MHz): δ 31.00, 35.62, 38.76, 122.87, 124.23, 124.26, 125.58, 125.71, 125.75, 128.22, 129.31, 130.89, 130.96, 131.29, 132.34, 132.52, 135.92, 139.32, 164.83. HRMS calculated for C₁₇H₁₅Cl₂F₃NOS (M+H)⁺ 408.02035, found 408.01914.

### N-[2-(3-Bromo-benzylsulfanyl)-ethyl]-2,6-dichloro-benzamide (51)

The compound was prepared using 2-(3-bromo-benzylsulfanyl)-ethylamine as the amine component and following the procedure described for 27: mp=74-76° C. ¹H NMR (CDCl₃, 400 MHz): δ 2.69 (t, J=6.4 Hz, 2H), 3.61 (q, J=6.4 Hz, 2H), 3.71 (s, 2H), 6.19 (bs, 1H), 7.17 (t, J=8.0 Hz, 1H), 7.23-7.38 (m, 5H), 7.49 (s, 1H). ¹³C NMR (CDCl₃, 100 MHz): δ 31.07, 35.48, 38.67, 122.88, 127.75, 128.29, 130.37, 130.59, 130.93, 132.02, 132.43, 135.97, 140.55, 164.75. HRMS calculated for C₁₆H₁₅BrCl₂NOS (M+H)⁺ 417.94347, found 417.94308.

### 2,6-Dichloro-N-[2-(2,3-dimethoxy-benzylsulfanyl)-ethyl]-benzamide (52)

The compound was prepared using 2-(2,3-dimethoxy-benzylsulfanyl)-ethylamine as the amine component and following the procedure described for 27: mp=108-110° C. ¹H NMR (CDCl₃, 400 MHz): δ 2.74 (t, J=6.4 Hz, 2H), 3.66 (q, J=6.4 Hz, 2H), 3.77 (s, 2H), 3.83 (s, 3H), 3.85 (s, 3H), 6.28 (bs, 1H), 6.80 (d, J=8.0 Hz, 1H), 6.90 (d, J=7.6 Hz, 1H), 6.99 (t, J=8.0 Hz, 1H), 7.22-7.31 (m, 3H). ¹³C NMR (CDCl₃, 100 MHz): δ 29.94, 31.09, 39.03, 55.93, 61.21, 111.68, 122.38, 124.32, 128.24, 130.84, 132.26, 132.47, 136.18, 147.27, 152.91, 164.69.

HRMS calculated for C₁₈H₂₀Cl₂NO₃S (M+H)⁺ 400.05409, found 400.05331.

### 2,6-Dichloro-N-[2-(3-iodo-benzylsulfanyl)-ethyl]-benzamide (53)

The compound was prepared using 2-(3-iodo-benzylsulfanyl)-ethylamine as the amine component and following the procedure described for 27: mp=75-78° C. ¹H NMR (CDCl₃, 400 MHz): δ 2.69 (t, J=6.4 Hz, 2H), 3.61 (q, J=6.4 Hz, 2H), 3.67 (s, 2H), 6.20 (bs, 1H), 7.03 (t, J=8.0 Hz, 1H), 7.03-7.32 (m, 4H), 7.56 (d, J=8.0 Hz, 1H), 7.69 (s, 1H). ¹³C NMR (CDCl₃, 100 MHz): δ 31.11, 35.38, 38.69, 94.75, 128.29, 128.38, 130.51, 130.93, 132.44, 135.99, 136.53, 137.94, 140.59, 164.73. HRMS calculated for C₁₆H₁₅Cl₂₁NOS (M+H)⁺ 465.92961, found 465.92940.

### 2,6-Dibromo-N-[2-(3-chloro-benzylsulfanyl)-ethyl]-benzamide (54)

The compound was prepared using 2,6-dibromobenzoic acid as the acid component and following the procedure described for 75: mp=55-57° C. ¹H NMR (CDCl₃, 400 MHz): δ 2.72 (t, J=6.4 Hz, 2H), 3.62 (q, J=6.4 Hz, 2H), 3.72 (s, 2H), 6.15 (bs, 1H), 7.10 (t, J=8.0 Hz, 1H), 7.22-7.23 (m, 3H), 7.34 (s, 1H), 7.51 (d, J=8.0 Hz, 2H). ¹³C NMR (CDCl₃, 100 MHz): δ 30.95, 35.54, 38.65, 120.66, 127.31, 127.68, 129.15, 130.10, 131.58, 131.97, 134.68, 139.75, 140.27, 166.57. HRMS calculated for C₁₆H₁₅Br₂ClNOS (M+H)⁺ 461.89296, found 461.88998.

### 2,6-Dibromo-N-[2-(3-bromo-benzylsulfanyl)-ethyl]-benzamide (55)

The compound was prepared following the procedure described for 75. 2,6-Dibromobenzoic acid and 2-(3-bromo-benzylsulfanyl)-ethylamine were used as acid and amine component respectively: mp=70-72° C. ¹H NMR (CDCl₃, 400 MHz): δ 2.68-2.73 (m, 2H), 3.57-3.62 (m, 2H), 3.71 (s, 2H), 6.23 (bs, 1H), 7.07-7.12 (m, 1H), 7.15-7.19 (m, 1H), 7.25-7.27 (m, 1H), 7.35-7.37 (m, 1H), 7.48-7.52 (m, 3H). ¹³C NMR (CDCl₃, 100 MHz): δ 30.93, 35.49, 38.69, 120.65, 122.88, 127.78, 130.39, 130.58, 131.57, 131.96, 132.04, 139.74, 140.56, 166.57. HRMS calculated for C₁₆H₁₅Br₃NOS (M+H)⁺ 505.84244, found 505.83892.

### N-[2-(3-Chloro-benzylsulfanyl)-ethyl]-2,6-dinitro-benzamide (56)

The compound was prepared following the procedure described for 27. 2,6-Dinitrobenzoyl chloride was used as acid chloride and 2-(3-chloro-benzylsulfanyl)-ethylamine as the amine component: mp=139-140° C. ¹H NMR (CDCl₃, 400 MHz): δ 2.74 (t, J=6.0 Hz, 2H), 3.61 (q, J=6.4 Hz, 2H), 3.71 (s, 2H), 6.35 (bs, 1H), 7.19-7.25 (m, 3H), 7.33 (s, 1H), 7.77 (t, J=8.0 Hz, 1H), 8.38 (d, J=8.0 Hz, 2H). ¹³C NMR (DMSO-d₆, 100 MHz): δ 29.74, 34.54, 39.60, 127.50, 127.92, 128.29, 129.34, 130.09, 130.87, 132.01, 133.62, 141.97, 147.92, 161.90. HRMS calculated for C₁₆H₁₅ClN₃O₅S (M+H)⁺ 396.04209, found 396.04216.

### 2,6-Dichloro-N-[2-(2,3-dihydroxy-benzylsulfanyl)-ethyl]-benzamide (58)

The compound was prepared from 52 using the procedure described for 29. Highly viscous liquid. ¹H NMR (CDCl₃, 400 MHz): δ 2.66 (t, J=6.4 Hz, 2H), 3.59 (q, J=6.4 Hz, 2H), 3.76 (s, 2H), 6.48 (s, 1H), 6.58-6.72 (m, 4H), 7.04 (bs, 1H), 7.17-7.26 (m, 3H). ¹³C NMR (CDCl₃, 100 MHz): δ 30.66, 39.15, 114.82, 120.82, 121.98, 124.30, 128.28, 131.15, 132.34, 135.43, 142.69, 144.86, 165.74. HRMS calculated for C₁₆H₁₆Cl₂NO₃S (M+H)⁺ 372.02279, found 372.02221.

### 2,6-Dichloro-N-[2-(3,5-dihydroxy-benzylsulfanyl)-ethyl]-benzamide (59)

The compound was prepared from 60 using the procedure described for 29: mp=55-57° C. ¹H NMR (acetone-d₆, 400 MHz): δ 2.68-2.72 (m, 2H), 3.55-3.59 (m, 2H), 3.65 (s, 2H), 6.23 (t, J=2.4 Hz, 1H), 6.36 (d, J=2.0 Hz, 2H), 7.35-7.42 (m, 3H), 7.93 (bs, 1H), 8.28 (s, 2H). ¹³C NMR (acetone-d₆, 400 MHz): δ 30.44, 35.62, 39.27, 107.64, 128.22, 130.99, 132.01, 136.89, 141.02, 158.73, 164.22. HRMS calculated for C₁₆H₁₆Cl₂NO₃S (M+H)⁺ 372.02279, found 372.02313.

### 2,6-Dichloro-N-[2-(3,5-dimethoxy-benzylsulfanyl)-ethyl]-benzamide (60)

The compound was prepared using 2-(3,5-dimethoxy-benzylsulfanyl)-ethylamine as the amine component and following the procedure described for 27: mp=46-47° C. ¹H NMR (CDCl₃, 400 MHz): δ 2.72 (t, J=6.4 Hz, 2H), 3.61 (q, J=6.4 Hz, 2H), 3.67 (s, 2H), 3.77 (s, 6H), 6.19 (bs, 1H), 6.31 (t, J=2.0 Hz, 1H), 6.48 (d, J=2.0 Hz, 2H), 7.22-7.31 (m, 3H). ¹³C NMR (CDCl₃, 100 MHz): δ 31.07, 36.32, 38.74, 55.56, 99.51, 106.93, 128.25, 130.88, 132.41, 136.05, 140.53, 161.10, 164.69. HRMS calculated for C₁₈H₂₀Cl₂NO₃S (M+H)⁺ 400.05409, found 400.05415.

### 2-Chloro-6-((2-(2,6-dichlorophenyl)-2-oxoethylthio)methyl)benzamide (61)

The compound was prepared from 62 using the procedure described for 45: mp=64-66° C. ¹H NMR (CDCl₃, 400 MHz): δ 2.67 (t, J=7.2 Hz, 2H), 3.45 (q, J=7.2 Hz, 2H), 3.77 (s, 2H), 6.29 (bs, 1H), 6.74 (s, 2H), 7.21-7.29 (m, 6H), 7.38-7.39 (m, 1H). ¹³C NMR (CDCl₃, 100 MHz): δ 30.89, 33.39, 39.63, 128.22, 128.63, 130.44, 130.77, 130.89, 132.29, 135.88, 136.07, 137.65, 164.99, 169.11. HRMS calculated for C₁₇H₁₆Cl₃N₂O₂S (M+H)⁺ 416.99980, found 417.00006.91% pure by HPLC.

### 2,6-Dichloro-N-[2-(3-chloro-2-cyano-benzylsulfanyl)-ethyl]-benzamide (62)

The compound was prepared using 2-(2-amino-ethylsulfanylmethyl)-6-chloro-benzonitrile as the amine component and following the procedure described for 27: mp=166-168° C. ¹H NMR (CDCl₃, 400 MHz): δ 2.78 (t, J=6.8 Hz, 2H), 3.71 (q, J=6.8 Hz, 2H), 3.95 (s, 2H), 6.33 (bs, 1H), 7.25-7.33 (m, 3H), 7.41-7.49 (m, 3H). ¹³C NMR (CDCl₃, 100 MHz): δ 31.39, 34.39, 38.92, 113.72, 115.11, 128.30, 128.51, 128.87, 130.99, 132.41, 133.73, 135.87, 137.86, 144.84, 164.89. HRMS calculated for C₁₇H₁₄Cl₃N₂OS (M+H)⁺ 398.98924, found 398.98777.

### N-[2-(3-Chloro-benzylsulfanyl)-ethyl]-benzamide (63)

The compound was prepared following the procedure described for 27. Benzoyl chloride was used as acid chloride and 2-(3-chloro-benzylsulfanyl)-ethylamine as the amine component: mp=75-76° C. ¹H NMR (CDCl₃, 400 MHz): δ 2.67 (t, J=6.4 Hz, 2H), 3.59 (q, J=6.4 Hz, 2H), 3.69 (s, 2H), 6.70 (bs, 1H), 7.18-7.26 (m, 3H), 7.33 (s, 1H), 7.39-7.43 (m, 2H), 7.47-7.51 (m, 1H), 7.74-7.76 (m, 2H). ¹³C NMR (CDCl₃, 100 MHz): δ 31.43, 35.55, 38.64, 127.18, 127.26, 127.64, 128.82, 129.11, 130.09, 131.80, 134.53, 134.64, 140.42, 167.78. HRMS calculated for C₁₆H₁₇ClNOS (M+H)⁺ 306.07194, found 306.07133.

### N-(2-Benzylsulfanyl-ethyl)-benzamide (64)

The compound was prepared following the procedure described for 27. Benzoyl chloride was used as acid chloride and 2-benzylsulfanyl-ethylamine as the amine component: mp=65-67° C. ¹H NMR (CDCl₃, 400 MHz): δ 2.67 (t, J=6.4 Hz, 2H), 3.58 (q, J=6.4 Hz, 2H), 3.73 (s, 2H), 6.65 (bs, 1H), 7.21-7.25 (m, 1H), 7.28-7.33 (m, 4H), 7.39-7.45 (m, 2H), 7.46-7.50 (m, 1H), 7.73-7.75 (m, 2H). ¹³C NMR (CDCl₃, 100 MHz): δ 31.33, 36.02, 38.59, 127.19, 127.44, 128.79, 128.88, 129.07, 131.75, 134.62, 138.29, 167.72. HRMS calculated for C₁₆H₁₈NOS (M+H)⁺ 272.11091, found 272.11101.

### N-(2-Benzylsulfanyl-ethyl)-2,6-dichloro-benzamide (65)

The compound was prepared using 2-benzylsulfanyl-ethylamine as the amine component and following the procedure described for 27: mp=96-97° C. ¹H NMR (CDCl₃, 400 MHz): δ 2.69 (t, J=6.4 Hz, 2H), 3.59 (q, J=6.4 Hz, 2H), 3.74 (s, 2H), 6.16 (bs, 1H), 7.19-7.33 (m, 8H). ¹³C NMR (CDCl₃, 100 MHz): δ 30.95, 36.04, 38.70, 127.45, 128.26, 128.87, 129.09, 130.89, 132.45, 136.07, 138.16, 164.71. HRMS calculated for C₁₆H₁₆Cl₂NOS (M+H)⁺ 340.03296, found 340.03318.

### 2,6-Dichloro-N-[2-(3-chloro-phenylmethanesulfonyl)-ethyl]-benzamide (66)

The compound was prepared from 1 following the procedure described for 30: mp=156-158° C. ¹H NMR (DMSO-d₆, 400 MHz): δ 3.29 (t, J=6.8 Hz, 2H), 3.63 (q, J=6.4 Hz, 2H), 4.62 (s, 2H), 7.37-7.49 (m, 7H), 8.99 (t, J=5.6 Hz, 1H). ¹³C NMR (DMSO-d₆, 100 MHz): δ 33.14, 50.92, 58.15, 128.79, 129.17, 130.49, 131.06, 131.47, 131.77, 131.93, 133.69, 136.59, 164.64. HRMS calculated for C₁₆H₁₅Cl₃NO₃S (M+H)⁺ 405.98382, found 405.98338.

### N-[2-(3-Chloro-benzylsulfanyl)-ethyl]-2,6-difluoro-benzamide (67)

The compound was prepared following the procedure described for 27. 2,6-difluorobenzoyl chloride was used as acid chloride and 2-(3-chloro-benzylsulfanyl)-ethylamine as the amine component: mp=41-43° C. ¹H NMR (CDCl₃, 400 MHz): δ 2.67 (t, J=6.4 Hz, 2H), 3.59 (q, J=6.4 Hz, 2H), 3.71 (s, 2H), 6.44 (bs, 1H), 6.90-6.96 (m, 2H) 7.19-7.26 (m, 3H), 7.32-7.39 (m, 2H). ¹³C NMR (CDCl₃, 100 MHz): δ 31.08, 35.34, 38.77, 112.11, 112.37, 114.27, 127.28, 127.64, 129.11, 130.06, 131.88, 131.99, 132.08, 134.64, 140.29, 158.88, 160.69, 161.40, 161.47. HRMS calculated for C₁₆H₁₅ClF₂NOS (M+H)⁺ 342.05309, found 342.05252.

### 2,6-Dichloro-N-[2-(3-cyclopropylcarbamoyl-benzylsulfanyl)-ethyl]-benzamide (68)

The compound was prepared from 44 following the procedure described for 78: mp=42-43° C. ¹H NMR (CDCl₃, 400 MHz): δ 0.35-0.38 (m, 2H), 0.47-0.49 (m, 2H), 2.48-2.52 (m, 2H), 2.71-2.73 (m, 1H), 3.58 (q, J=6.4 Hz, 2H), 3.74 (s, 2H), 7.18-7.37 (m, 5H), 7.45 (d, J=7.6 Hz, 1H), 7.67-7.75 (m, 3H). ¹³C NMR (CDCl₃, 100 MHz): δ 5.80, 23.41, 28.25, 34.68, 39.41, 127.07, 127.69, 128.13, 129.35, 130.91, 132.03, 132.21, 134.36, 135.71, 138.21, 165.37, 169.05. HRMS calculated for C₂₀H₂₁Cl₂N₂O₂S (M+H)⁺ 423.07008, found 423.07114.

### 2,6-Dichloro-N-[2-(3-chloro-phenylmethanesulfinyl)-ethyl]-benzamide (71)

The compound was prepared from 1 following the procedure described for 32: mp=174-176° C. ¹H NMR (DMSO-d₆, 400 MHz): δ 2.74-2.79 (m, 1H), 2.97-3.00 (m, 1H), 3.52-3.55 (m, 1H), 3.59-3.61 (m, 1H), 4.07 (d, J=12.8 Hz, 1H), 4.23 (d, J=12.8 Hz, 1H), 7.27-7.29 (m, 1H), 7.38-7.41 (m, 4H), 7.45-7.47 (m, 2H), 8.98 (t, J=5.6 Hz, 1H). ¹³C NMR (DMSO-d₆, 100 MHz): δ 33.60, 50.71, 56.49, 128.45, 128.77, 129.71, 130.69, 130.99, 131.78, 133.68, 134.44, 136.81, 164.55. HRMS calculated for C₁₆H₁₅Cl₃NO₂S (M+H)⁺ 389.98890, found 389.98985.

### 2,6-Dichloro-N-[2-(3-chloro-benzyloxy)-ethyl]-benzamide (72)

The compound was prepared using 2-(3-chloro-benzyloxy)-ethylamine as the amine component and following the procedure described for 27: mp=70-72° C. ¹H NMR (CDCl₃, 400 MHz): δ 3.66-3.73 (m, 4H), 4.50 (s, 2H), 6.26 (bs, 1H), 7.18-7.32 (m, 7H). ¹³C NMR (CDCl₃, 100 MHz): δ 39.89, 69.18, 72.64, 125.94, 127.95, 128.17, 128.28, 129.98, 130.87, 132.43, 134.60, 136.14, 140.09, 164.75. HRMS calculated for C₁₆H₁₅Cl₃NO₂ (M+H)⁺ 358.01684, found 358.01726.

### 2,6-Dichloro-N-[2-(3-chloro-5-methoxy-benzylsulfanyl)-ethyl]-benzamide (73)

The compound was prepared using 2-(3-chloro-5-methoxy-benzyloxy)-ethylamine as the amine component and following the procedure described for 27: mp=83-84° C. ¹H NMR (CDCl₃, 400 MHz): δ 2.69 (bs, 2H), 3.59 (bs, 2H), 3.66 (s, 2H), 3.78 (s, 3H), 6.33 (bs, 1H), 6.76 (s, 2H), 6.91 (s, 1H), 7.27 (s, 3H). ¹³C NMR (CDCl₃, 100 MHz): δ 31.05, 35.59, 38.61, 55.77, 113.27, 121.39, 128.15, 128.25, 130.90, 132.38, 135.16, 135.97, 141.15, 160.58, 164.76. HRMS calculated for C₁₇H₁₇Cl₃NO₂S (M+H)⁺ 404.00455, found 404.00305.

### N-[2-(3-Chloro-benzylsulfanyl)-ethyl]-2,6-bis-trifluoromethyl-benzamide (74)

The compound was prepared following the procedure described for 27. 2,6-Bis-trifluoromethylbenzoyl chloride was used as acid chloride and 2-(3-chloro-benzylsulfanyl)-ethylamine as the amine component. The reaction was carried out at refluxing condition. mp=93-94° C. ¹H NMR (CDCl₃, 400 MHz): δ 2.64 (t, J=6.8 Hz, 2H), 3.58 (q, J=6.8 Hz, 2H), 3.69 (s, 2H), 6.28 (bs, 1H), 7.19-7.22 (m, 3H), 7.32 (s, 1H), 7.66 (t, J=8.4 Hz, 1H), 7.89 (d, J=8.0 Hz, 2H). ¹³C NMR (CDCl₃, 100 MHz): δ 30.44, 35.36, 38.86, 121.79, 124.53, 127.27, 127.65, 128.83, 129.08, 129.15, 129.47, 129.79, 130.02, 130.06, 130.12, 130.17, 134.02, 134.66, 140.18, 164.38. HRMS calculated for C₁₈H₁₅ClF₆NOS (M+H)⁺ 442.04670, found 442.04687.

### 2,6-Dichloro-N-[2-(2-chloro-benzylsulfanyl)-ethyl]-benzamide (76)

The compound was prepared using 2-(2-chloro-benzylsulfanyl)-ethylamine as the amine component and following the procedure described for 27: mp=110-112° C. ¹H NMR (CDCl₃, 400 MHz): δ 2.75 (t, J=6.4 Hz, 2H), 3.65 (q, J=6.4 Hz, 2H), 3.87 (s, 2H), 6.28 (bs, 1H), 7.16-7.32 (m, 5H), 7.34-7.39 (m, 2H). ¹³C NMR (CDCl₃, 100 MHz): δ 31.31, 33.53, 38.89, 127.22, 128.26, 128.91, 130.09, 130.90, 131.12, 132.44, 134.14, 135.93, 136.01, 164.73. HRMS calculated for C₁₆H₁₅Cl₃NOS (M+H)⁺ 373.99399, found 373.99368.

### 2,6-Dichloro-N-[2-(4-chloro-benzylsulfanyl)-ethyl]-benzamide (77)

The compound was prepared using 2-(4-chloro-benzylsulfanyl)-ethylamine as the amine component and following the procedure described for 27: mp=104-106° C. ¹H NMR (CDCl₃, 400 MHz): δ 2.67 (t, J=6.8 Hz, 2H), 3.59 (q, J=6.4 Hz, 2H), 3.71 (s, 2H), 6.25 (bs, 1H), 7.22-7.31 (m, 7H). ¹³C NMR (CDCl₃, 100 MHz): δ 30.86, 35.29, 38.71, 128.28, 128.99, 130.44, 130.93, 132.39, 133.19, 135.90, 136.71, 169.75. HRMS calculated for C₁₆H₁₅Cl₃NOS (M+H)⁺ 373.99399, found 373.99380.

### Drug Library Screen and Lead Hit Selection

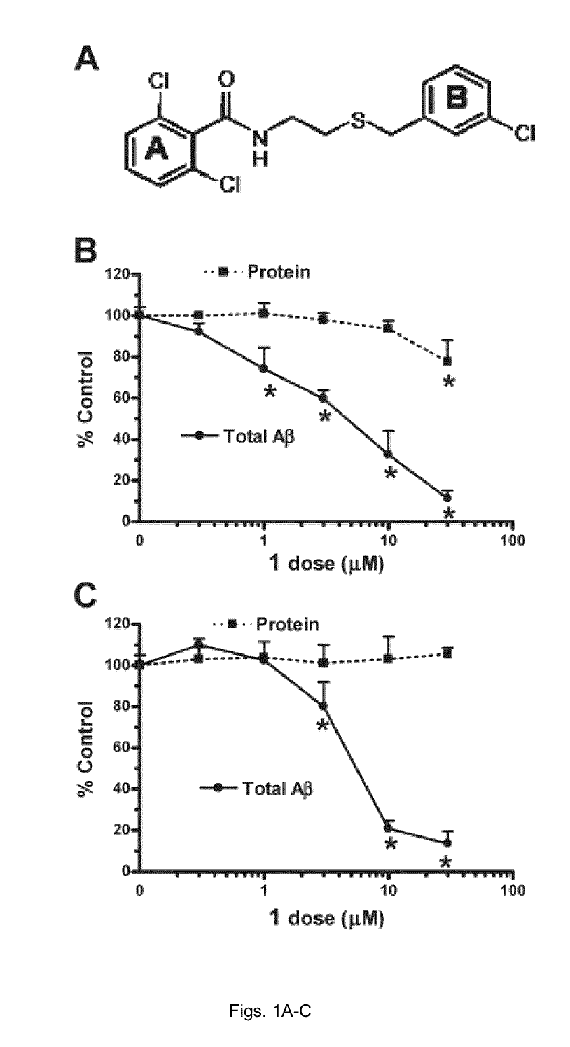

We screened 17200 small drug-like molecules obtained from ChemBridge (San Diego, Calif.) for their ability to inhibit Aβ secretion into media conditioned by stably transfected H4βAPP695 wt human CNS derived cells. The initial screen was performed in 96-well plates with drugs distributed by the hanging drop method yielding estimated drug levels of 10-25 μM. Total Aβ levels in the 24 h conditioned media were measured by a sandwich ELISA assay, and cytotoxicity was assessed by methylene blue staining of the cells in the wells, as previously described. After assay validation, we screened each compound in a single well and identified 80 compounds that both reduced Aβ levels in the conditioned media by at least 40%, and did not reduce methylene blue staining by 25% or more. Each of the 80 drugs was rescreened in duplicate, and 29 positive hits were individually ordered and validated in dose response studies, as previously described, yielding a confirmed hit rate of 0.17% of the screened compounds. We selected 1 (FIG. 1A, ChemBridge no. 5538506) as the lead hit for three reasons: (1) It displayed Aβ lowering activity at 1 μM in H4βAPP695 wt cells (FIG. 1B), (2) it did not have significant cytotoxicity, measured by total cell protein content, at 10 μM (FIG. 1B), and (3) it had the lowest mi Log P value of 5.165, an indicator of hydrophobicity associated with blood-brain barrier penetration, of the hits with similar potency. Compound 1 was also effective in reducing secreted Aβ levels in cultured primary neurons derived from APP transgenic mice (FIG. 1C).

### Mechanism of Action of Lead Hit

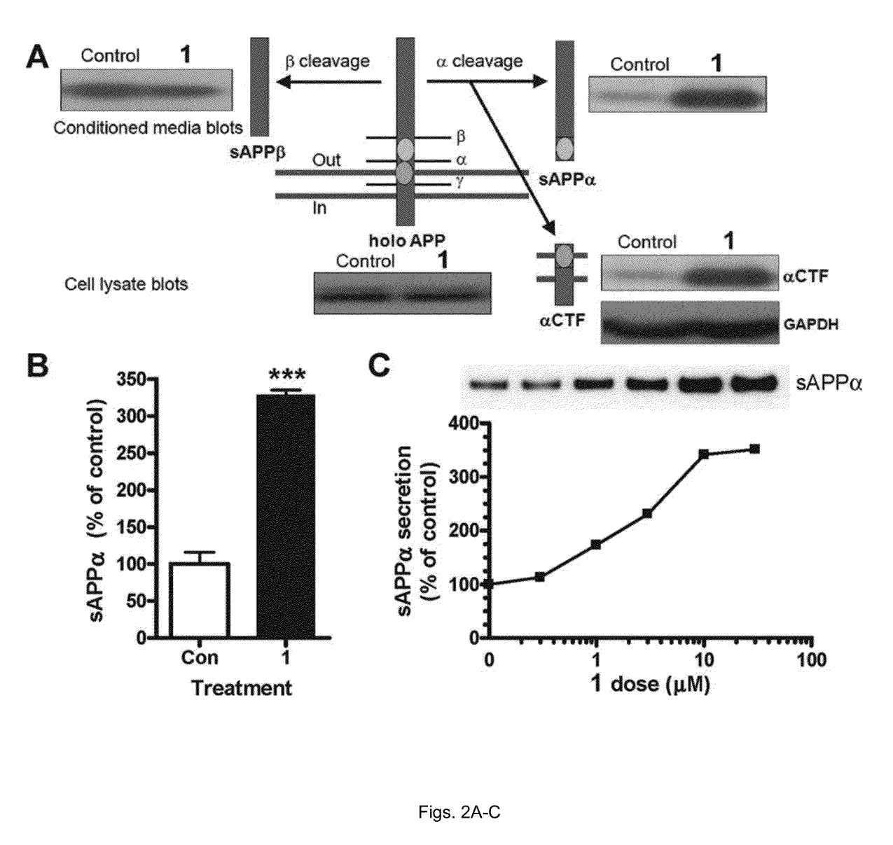

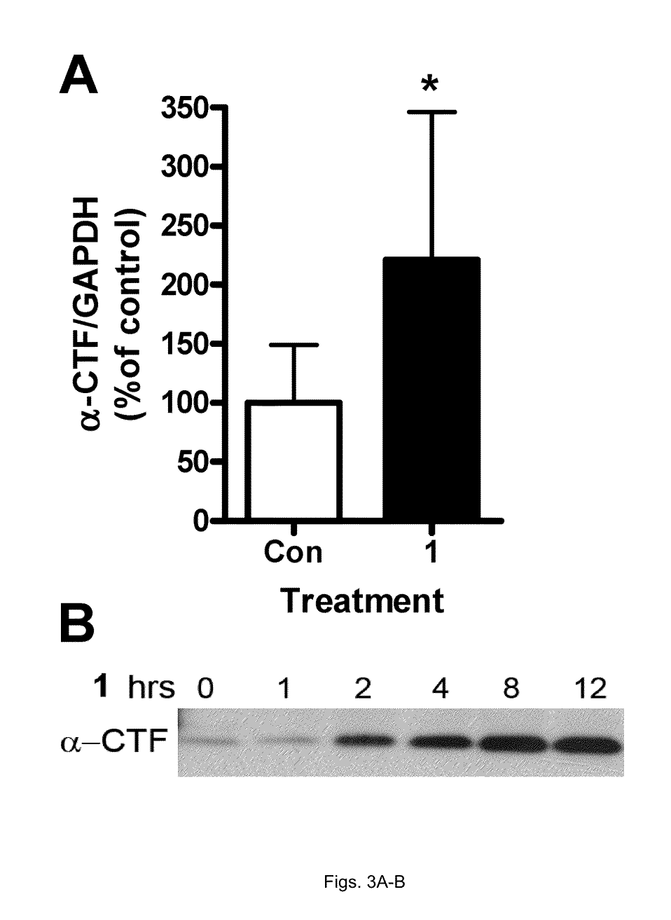

The alternate processing of holoAPP by either the β-secretase (a precursor to Aβ production) or α-secretase (which clips in the middle of the Aβ region) is shown in FIG. 2A. Compound 1 induced decrease in total Aβ secretion may be due to its effect on APP processing or due to decreased production of its precursor APP. To probe its mechanism, H4βAPP695 wt cells were treated with 10 μM 1 for 24 h. Using Western blot assays, we measured secreted APPα (sAPPα) and APPβ (sAPPβ) in the conditioned media, and full-length holo-APP and the 83 residue long α-C-terminal fragment (α-CTF) in cell lysate. Compound 1 caused an increase in sAPPα and α-CTF level without altering sAPPβ or cellular full length APP (FIG. 2A). Compound 1 treatment significantly increased sAPPα levels by over 3-fold, with a dose response similar to that observed for inhibition of Aβ secretion (FIG. 2B, C). Compound 1 also raised cellular α-CTF levels by over 2-fold (FIG. 3A), an effect that was observed in as little as 2 h after addition of compound 1, and reached maximal levels by 8 h (FIG. 3B). The effect of a 4 h treatment with compound 1 on the increase in cellular of α-CTF was not blocked by the protein synthesis inhibitor cyclohexamide, indicating that new protein synthesis was not required for this effect (data not shown). Thus, 1 appears to decrease Aβ secretion by activating the α-secretase pathway, a favorable pathway as the large secreted sAPPα protein is reported to have neurotrophic and neuroprotective activities.

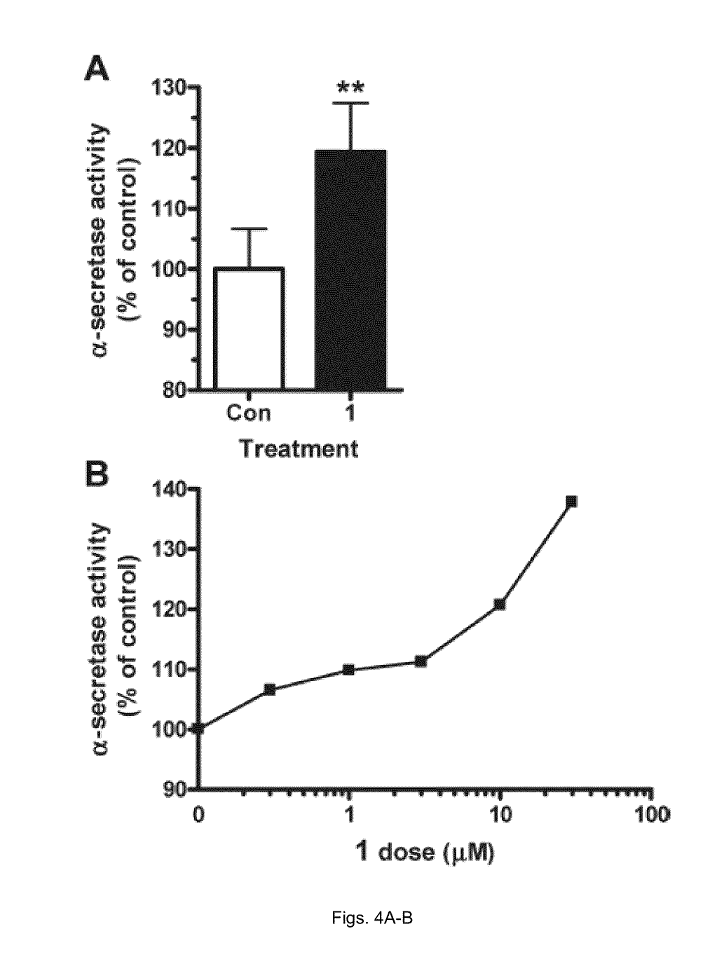

We measured the activity of α-secretase using a fluorogenic peptide substrate in lysates of H4βAPP695 wt cells. Pretreatment of the cells with 10 μM of compound 1 for 24 h led to a significant 20% increase in α-secretase activity (FIG. 4A), with a dose response similar to that observed for inhibition of Aβ secretion (FIG. 4B). However, addition of compound 1 directly to the cell lysates, rather than to the live cells, had no effect on α-secretase activity (data not shown), implying an indirect cellular activation of this pathway rather than direct activation of the α-secretase protease by compound 1.

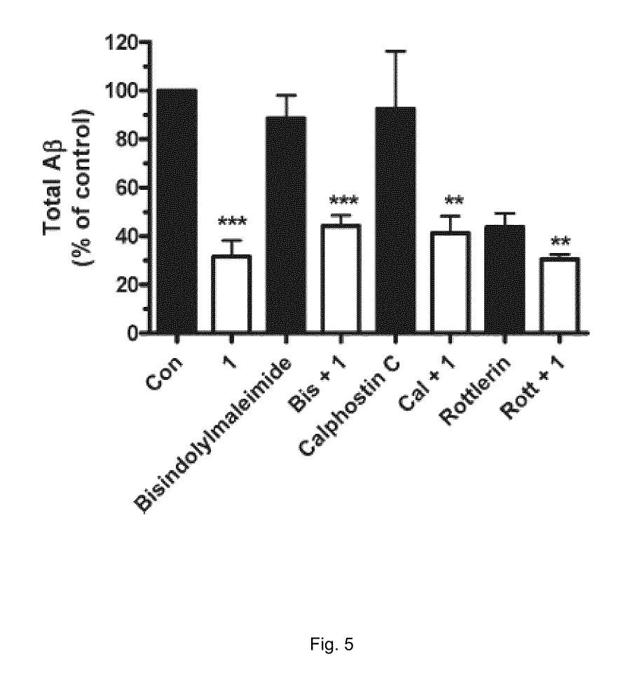

The tumor promoter phorbal myristate acetate is an activator of protein kinase C (PKC) that has been reported to activate α-secretase activity. Thus, we wanted to determine if protein kinase C inhibitors could block the effect of compound 1 on Aβ secretion. The PKC inhibitors Bisindolylmaleimide I and Calphostin C had no effect on basal or compound 1 inhibited Aβ secretion, while the PKC inhibitor Rottlerin itself reduced Aβ secretion but did not significantly alter the effect of compound 1 (FIG. 5).

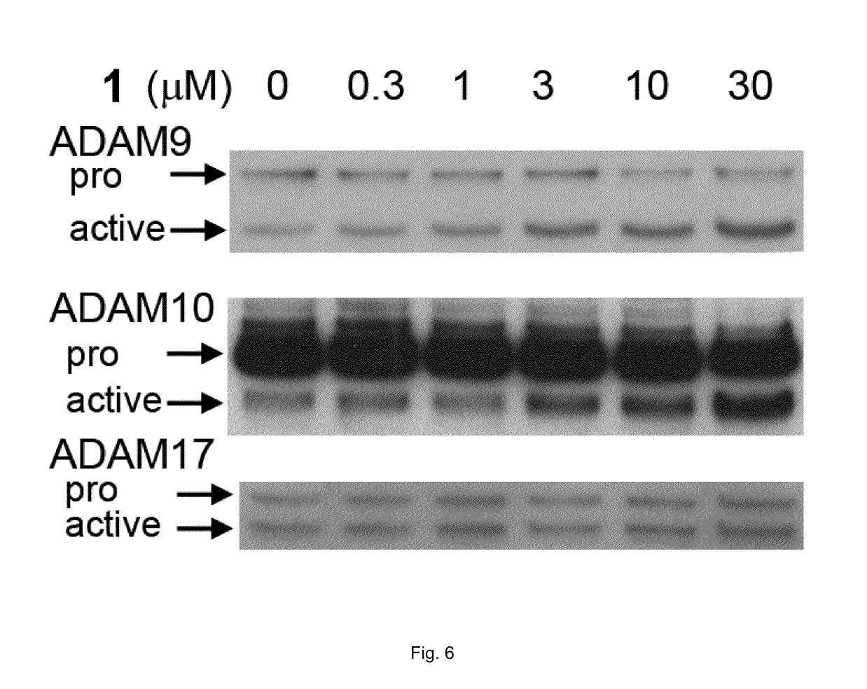

Several members of the ADAM (a disintegrin and metalloproteinase) family, ADAM9, ADAM10, and ADAM17, have been reported to have α-secretase activity. These proteases are made in inactive pro forms and must be proteolytically cleaved into their active forms. Using antibodies specific for these three ADAM proteases, we assessed the levels of the pro and active forms in cells cultured with increasing concentrations of compound 1. We observed a dose dependent increase in active ADAM9 and ADAM10 but did not observe much effect on the active form of ADAM17 (FIG. 6). At the 10 μM dose of compound 1, we observed a 31% increase in the ratio of active/pro ADAM9 (p<0.05), N=3 each control and compound 1) and a 42% increase in the ratio active/pro ADAM10 (p<0.05). Although the direct target for compound 1 has not been identified, it seems likely that its mechanism is via the increased ADAM 9 and ADAM10 processing into active α-secretase, which cleaves APP in a manner that precludes Aβ production.

### Commercial Analogue Evaluation

We searched for compounds having at least 60% similarity to compound 1 using the ChemBridge Hit2lead.com and PubChem Web sites. We were able to obtain 24 compounds (compounds 2-25, described above) commercially which were tested for their ability to decrease Aβ secretion from H4βAPP695 wt cells. Each drug, along with compound 1, was tested in 1-4 independent experiments, each in quadruplicate, at the 10 μM dose. Table 1 lists compound 1 and the 24 analogues, their source, product number, molecular weight, and the mean % reduction in Aβ secretion compared to the 0.02% DMSO vehicle control. In these experiments, 10 μM of compound 1 yielded a 75.3±11.6% decrease in Aβ secretion (N=4±SD). None of the 24 analogues were as effective as 1. Visual inspection of these compounds revealed that removing one of the two ortho-chlorines on aryl region A led to a large loss in activity.

TABLE 1
Commercial Analogues Inhibition of Aβ Secretion

| Analogue | MW | % β Decrease | Vendor** | Product # |
|---|---|---|---|---|
| 1 | 375 | 75.3 | C | 5538506 |
| 2 | 375 | 5.3 | C | 6460634 |
| 3 | 340 | 4.7 | C | 6478444 |
| 4 | 454 | 7.9 | C | 5333392 |
| 5 | 375 | 0.7 | C | 6757682 |
| 6 | 409 | 4.3 | C | 6842391 |
| 7 | 275 | 15.8 | C | 5736522 |
| 8 | 262 | 6.9 | C | 5730201 |
| 9 | 290 | 10.3 | C | 5924186 |
| 10 | 303 | 5.1 | C | 5739287 |
| 11 | 292 | −4.4 | C | 6465442 |
| 12 | 264 | 3.8 | C | 7272827 |
| 13 | 406 | 7.6 | C | 7243118 |
| 14 | 434 | 4.0 | C | 7287552 |
| 15 | 261 | 6.6 | C | 7478133 |
| 16 | 340 | 4.0 | C | 6399827 |
| 17 | 340 | −12.0 | C | 6408972 |
| 18 | 324 | 6.4 | C | 7739577 |
| 19 | 386 | 37.0 | R | 9M-709 |
| 20 | 409 | 39.8 | C | 6698964 |
| 21 | 458 | 47.8 | C | 7991391 |
| 22 | 342 | 27.3 | M | SEW06618 |
| 23 | 375 | 5.3 | M | SEW06549 |
| 24 | 374 | 39.2 | M | SEW06625 |
| 25 | 332 | 0.2 | C | 5328293 |

*All drugs tested at 10 μM, % decrease relative to vehicle treated control cells.
**Vendors: C, ChemBridge; M, Maybridge; R, Ryan Scientific.

### Design, Synthesis, and Evaluation of Novel Analogues

Fifty-three novel analogues (compounds 26-78 described above) were synthesized to examine their efficacy in reducing Aβ secretion. In examining compound 1, the molecule can be parsed into three regions: aryl region A, linker region, and aryl region B. These regions along with our synthetic approach to constructing this small library are shown in FIG. 8. We then took the strategy of iterative modification of the three regions starting from commercially available benzyl halides (III). Conversion of III to the corresponding β-benzylmercaptoethylamine (β-BMEA) derivatives (II) gave derivatives that could further be reacted with substituted benzoyl chlorides, giving the desired amides (I).

In general, various β-BMEA derivatives were synthesized from the corresponding benzyl halides using our newly developed procedure. The commercially unavailable benzyl halides were synthesized from the corresponding benzyl alcohols or toluene derivatives, whereas the commercially unavailable 2,6-disubstituted benzoyl chlorides were synthesized from the corresponding acid derivatives (FIG. 9). In particular, 2,6-dibromobenzoic acid was synthesized from 2,6-dibromotoulene using a KMNO₄ mediated oxidation strategy.

By replacing 2,6-dichlorobenzoyl moiety of aryl region A, we synthesized seven new analogues (compounds 36, 54, 56, 63, 67, 74, and 75), whereas the replacement of 3-chloro moiety of aryl region B gave us a library of 30 new analogues (compounds 26-29, 38-41, 43-45, 47-53, 58-62, 65, 68-70, 73, 76, and 77). Modification at the linker region yielded five new analogues (compounds 57, 66, 71, 72, and 78). Finally, on the basis of initial findings in conjunction with the above chemistry, several analogues were synthesized whereby more than one region was diversified, yielding an additional 11 analogues (compounds 30-35, 37, 42, 46, 55, and 64). Although the majority of the molecules synthesized followed the route outlined in Scheme 2, several required specific manipulations.

The compounds and their effects on reducing Aβ secretion and total protein levels (cytotoxicity) are shown in Table 2.

TABLE 2
Newly Synthesized Analogues and Inhibition of Aβ Secretions^{a}

| | | | Total | | | | Cell protein (% | | | |
|---|---|---|---|---|---|---|---|---|---|---|
| | | | Aβ (% decrease) | | | | decrease) | | | |
| | MW | | 10 μM | | 30 μM | | 10 μM | | 30 μM | |
| Compound | (MP) | miLogP | Mean | SD | Mean | SD | Mean | SD | Mean | SD |
| 26 | 370 | 4.45 | 1.6 | 14.0 | 1.1 | 1 | 6.7 | 10.1 | 12.7 | 1.1 |
| | (80) | | | | | | | | | |
| 27 | 370 | 3.56 | 60.6 | 5.0 | 73.9 | 3.7 | 8.4 | 8.8 | 13.2 | 8.3 |
| | (60) | | | | | | | | | |
| 28 | 356 | 3.93 | 13.1 | 4.0 | 56.7 | 4.1 | 8.7 | 5.5 | 4.4 | 4.1 |
| | (138) | | | | | | | | | |
| 29 | 356 | 5.03 | 56.4 | 4.5 | 67.7 | 6.8 | 6.3 | 1.6 | 6.6 | 6.34 |
| | (108) | | | | | | | | | |
| 30 | 402 | 4.40 | 5.3 | 21.5 | 4.9 | 9.8 | 1.2 | 8.0 | 8.9 | 4.9 |
| | (172) | | | | | | | | | |
| 31 | 386 | 3.31 | −10.1 | 22.5 | 9.6 | 8.2 | 7.7 | 19.1 | 4.4 | 7.4 |
| | (144) | | | | | | | | | |
| 32 | 372 | 3.62 | −13.6 | 11.0 | −15.8 | 23 | 1.6 | 7.7 | 9.2 | 4.9 |
| | (165) | | | | | | | | | |
| 33 | 361 | 4.65 | −23.7 | 27.8 | 15.9 | 11 | 2.8 | 3.7 | 4.4 | 7.4 |
| | (41) | | | | | | | | | |
| 34 | 319 | 4.94 | 6.2 | 27.1 | −0.6 | 12 | 0.5 | 16.7 | 3.9 | 6.7 |
| | (60) | | | | | | | | | |
| 35 | 366 | 3.83 | −22.5 | 17.9 | −25.2 | 23 | 10.5 | 9.6 | 14.3 | 5.6 |
| | (90) | | | | | | | | | |
| 36 | 391 | 5.59 | −13.1 | 18.6 | 11.2 | 7.3 | 7.1 | 5.2 | −0.6 | 6.3 |
| | (69) | | | | | | | | | |
| 37 | 365 | 5.30 | −14.1 | 7.0 | −2.8 | 12 | 3.7 | 7.7 | −3.8 | 10.8 |
| | (123) | | | | | | | | | |
| 38 | 398 | 4.43 | 20 | 8.9 | 38.7 | 6.7 | −4.8 | 4.8 | 4.3 | 12.3 |
| | (126) | | | | | | | | | |
| 39 | 385 | 5.57 | −1.5 | 13.8 | 1.6 | 12 | 9.2 | 13.6 | 5.9 | 15.1 |
| | (76) | | | | | | | | | |
| 40 | 355 | 4.45 | 58.5 | 6.9 | 72.5 | 4.5 | 7.5 | 14.0 | 24.6 | 7.1 |
| | (55) | | | | | | | | | |
| 41 | 366 | 3.56 | 0.1 | 7.8 | 17 | 11 | 15.3 | 9.7 | 13.1 | 12.1 |
| | (58) | | | | | | | | | |
| 42 | 412 | 3.92 | −2.9 | 16.0 | −4.7 | 16 | 1.9 | 21.4 | 9.3 | 9.5 |
| | (43) | | | | | | | | | |
| 43 | 384 | 5.03 | 3 | 16.0 | 10.1 | 13 | 5.8 | 17.4 | 5.4 | 9 |
| | (165) | | | | | | | | | |
| 44 | 383 | 4.40 | −11.4 | 22.2 | −3.8 | 15 | 7.1 | 15.6 | 3.2 | 13.7 |
| | (148) | | | | | | | | | |
| 45 | 325 | 3.31 | 6.2 | 15.1 | 32.3 | 8.3 | −2.8 | 19.7 | −8.6 | 15.2 |
| | (39) | | | | | | | | | |
| 46 | 358 | 3.62 | −16.8 | 12.9 | −15.5 | 8.6 | 0.1 | 12.1 | 1.8 | 7.7 |
| | (70) | | | | | | | | | |
| 47 | 354 | 4.65 | 10.2 | 30.1 | 36.1 | 8.6 | 2.6 | 9.2 | 3.7 | 6.6 |
| | (56) | | | | | | | | | |
| 48 | 369 | 4.94 | 70.5 | 4.3 | 85.8 | 4.2 | 11.7 | 5.6 | 16.6 | 8.9 |
| | (63) | | | | | | | | | |
| 49 | 408 | 3.83 | 19.9 | 12.0 | 36.4 | 11 | 1.5 | 12.8 | −2.1 | 11.5 |
| | (54) | | | | | | | | | |
| 50 | 419 | 5.59 | 73.9 | 4.7 | 93.4 | 2.9 | 19 | 6.5 | 47.8 | 15.4 |
| | (74) | | | | | | | | | |
| 51 | 400 | 5.30 | 75 | 3.6 | 90.1 | 3.1 | 12.3 | 9.2 | 38.6 | 12 |
| | (108) | | | | | | | | | |
| 52 | 466 | 4.43 | 57.8 | 16.3 | 72.8 | 8.3 | −0.1 | 10.3 | 8.7 | 5.8 |
| | (75) | | | | | | | | | |
| 53 | 463 | 5.57 | 84.4 | 5.3 | 88.3 | 6.4 | 9.2 | 7.5 | 19 | 7.7 |
| | (55) | | | | | | | | | |
| 54 | 508 | 5.43 | 74.2 | 11.8 | 83.4 | 6.7 | 6.7 | 8.3 | 20.8 | 5.3 |
| | (70) | | | | | | | | | |
| 55 | 395 | 5.56 | 78.6 | 6.4 | 87.3 | 5.9 | 9.8 | 7.1 | 23.9 | 6.6 |
| | (139) | | | | | | | | | |
| 56 | 361 | 3.73 | 28.8 | 18.2 | 52.9 | 15 | 0.1 | 9.3 | 17.8 | 7.6 |
| | (liq) | | | | | | | | | |
| 57 | 372 | 5.53 | 45.2 | 15.6 | 45.6 | 21 | −3.6 | 11.3 | 6.6 | 10.8 |
| | (liq) | | | | | | | | | |
| 58 | 372 | 3.75 | −4.3 | 22.1 | 33.1 | 8.2 | −4.8 | 21.0 | 14 | 9.8 |
| | (liq) | | | | | | | | | |
| 59 | 372 | 3.48 | 3.1 | 13.0 | 17.2 | 13 | −0.4 | 12.9 | −0.7 | 10.4 |
| | (55) | | | | | | | | | |
| 60 | 400 | 4.55 | 47.9 | 6.3 | 80.4 | 4.1 | 2 | 9.8 | −8.7 | 22.6 |
| | (46) | | | | | | | | | |
| 61 | 417 | 3.91 | 44.7 | 10.6 | 49.7 | 6.1 | 2.5 | 12.8 | −5.6 | 16.2 |
| | (64) | | | | | | | | | |
| 62 | 400 | 5.06 | 48.7 | 6.8 | 52.3 | 14 | 3.5 | 9.7 | 1.3 | 10.7 |
| | (166) | | | | | | | | | |
| 63 | 306 | 3.91 | 0.3 | 17.6 | 10.7 | 11 | 2.4 | 10.8 | −4.5 | 15.4 |
| | (75) | | | | | | | | | |
| 64 | 271 | 3.25 | −5.7 | 16.4 | 5.5 | 11 | 0.7 | 9.7 | −4.1 | 11.2 |
| | (65) | | | | | | | | | |
| 65 | 340 | 4.51 | 51.6 | 11.7 | 58.6 | 12 | −1.1 | 12.9 | −0.7 | 10.2 |
| | (96) | | | | | | | | | |
| 66 | 407 | 4.04 | 8.9 | 14.0 | 9.3 | 16 | −2.2 | 10.2 | −3.8 | 12.2 |
| | (156) | | | | | | | | | |
| 67 | 342 | 4.14 | 4.9 | 9.9 | 21.6 | 7.7 | 5.1 | 8.8 | 8.1 | 8.6 |
| | (41) | | | | | | | | | |
| 68 | 423 | 4.05 | 1.9 | 7.2 | 11 | 9.9 | 0 | 4.8 | −1 | 9 |
| | (42) | | | | | | | | | |
| 69 | 423 | 4.54 | −21.4 | 34.7 | −2 | 31 | 3 | 11.7 | 3.2 | 20.3 |
| | (46) | | | | | | | | | |
| 70 | 444 | 3.71 | 17.4 | 16.8 | 24.3 | 9.8 | −7.3 | 20.5 | 6.1 | 9.8 |
| | (liq) | | | | | | | | | |
| 71 | 391 | 3.64 | 4 | 21.7 | −16.5 | 11 | −4.9 | 10.2 | 7.3 | 2.7 |
| | (174) | | | | | | | | | |
| 72 | 359 | 4.62 | 10.5 | 18.7 | −5.1 | 14 | −9.9 | 18.4 | 4.2 | 4.4 |
| | (70) | | | | | | | | | |
| 73 | 405 | 5.17 | 67.7 | 5.4 | 81 | 1.8 | 0.9 | 10.3 | 0.4 | 12.1 |
| | (83) | | | | | | | | | |
| 74 | 442 | 6.02 | 6.2 | 13.6 | 13.5 | 12 | −4.2 | 18.6 | −0.4 | 9.8 |
| | (93) | | | | | | | | | |
| 75 | 339 | 4.71 | 29.5 | 5.0 | 51.6 | 14 | 0.9 | 9.3 | −9.4 | 22.9 |
| | (53) | | | | | | | | | |
| 76 | 375 | 5.14 | 75.6 | 4.3 | 80 | 4 | −0.5 | 11.1 | −11 | 29.6 |
| | (110) | | | | | | | | | |
| 77 | 375 | 5.19 | 21.8 | 9.0 | 36.5 | 13 | −6.4 | 17.6 | 3 | 14.9 |
| | (104) | | | | | | | | | |
| 78 | 389 | 5.26 | 6.7 | 9.8 | 13.5 | 24 | −1.3 | 4.9 | −.07 | 16.5 |
| | (116) | | | | | | | | | |

^{a}MP, melting point given in ° C.; liq denotes a viscous liquid at room temperature. 30 and 66 were crystallized from ethanol.

Thirteen compounds (compounds 27, 29, 40, 48, 50, 51, 52, 53, 54, 55, 65, 73, and 76) at a 10 μM dose had roughly similar efficacy as 1 (>50% inhibition of Aβ secretion), but five of these (compounds 40, 50, 51, 54, and 55) displayed cytotoxicity with a decrease in cell protein >20% at the 30 μM dose. Inspection of these 13 active analogues reveals some flexibility of substitutions on aryl region B. Replacing the meta-chorine with meta-methyl ether (27), hydroxyl (29), nitrate (40), methyl (48), carbon trifluoride (50), bromine (51), or iodine (53) all yielded good activity. Other substitutions on aryl region B meta position did not yield good activity such as cyanate (38), amine (41), carboxylate (44), fluorine (47), formyl (49), and other more bulky substitutions (compounds 39, 43, 45, 68, 69, 70). Removing the aryl region B meta-chlorine (65) retained activity, as did substituting an ortho-chlorine (76); however, substitutions with para-chlorine (77), para-methyl ether (26), or para-hydroxyl (28) led to loss of activity. While an aryl region B with two substituents could be tolerated, such as meta-chlorine meta-methyl ether (73), meta-meta-methyl ether (60), ortho-meta-methyl ether (52), and ortho-amide meta-chlorine (61), other compounds with two substitutions such as meta-meta-hydroxyl (59) and ortho-meta-hydroxyl (58) lost activity. On aryl region A, substitutions for one or both of the two chlorines in the ortho-positions, or their removal, were not well tolerated (compounds 34, 35, 36, 37, 56, 63, 67, 74, and 75), with the exception of bromine substitution, which maintained activity (compounds 54 and 55). No tested substitutions in the linker region were well tolerated (compounds 30, 31, 32, 33, 57, 66, 71, 72, and 78).

To screen for resistance to metabolism, 1 and eight noncytotoxic analogues with >50% Aβ reducing activity (compounds 27, 29, 48, 52, 53, 65, 73, and 76) were incubated at 20 μM for 24 h with or without THLE-3 human hepatoma cells. This media was diluted 1:1 with fresh media (drug concentration diluted to 10 μM) and used to treat H4βAPP695 wt cells for 24 h to assay inhibition of Aβ secretion. Table 3 shows that compound 1 and most of the active analogues maintained about 50% of their Aβ inhibiting activity during the 24 h incubation with the hepatoma cells, with the exception of compound 52, which lost virtually all of its activity. The three analogues with the most net activity after incubation with the hepatoma cells were compounds 76, 53, and 48 with 49%, 40%, and 38% inhibition of Aβ secretion, respectively.

TABLE 3
Drug Activity Persistence after Incubation with Hepatoma Cells
hepatoma incubation^{a}

| | No total Aβ | Yes total Aβ | % remaining |
|---|---|---|---|
| compd | (% decrease) | (% decrease) | Activity^{b} |
| 1 | 69.4 | 34.6 | 50 |
| 27 | 60.6 | 22.2 | 37 |
| 29 | 56.4 | 23.1 | 41 |
| 48 | 70.5 | 38.3 | 54 |
| 52 | 57.8 | 1.9 | 3 |
| 53 | 84.4 | 40.0 | 47 |
| 65 | 51.6 | 27.0 | 52 |
| 73 | 67.7 | 33.5 | 49 |
| 76 | 75.6 | 48.8 | 65 |

^{a}Drugs incubated with H4 cells at 10 μM, with or without preincubation with hepatoma cells for 24 h.

To screen for bioavailability of compound 1 and seven active noncytotoxic analogues (compounds 27, 48, 52, 53, 65, 73, and 76), we assessed their transcellular transport through confluent monolayers of Caco-2 cells grown on transwell inserts. Monolayer integrity was confirmed by high levels of transcellular electrical resistance (>160 ohms/cm²) and by transcellular impermeability to the fluorescent dye lucifer yellow. The compounds were placed in the upper chamber, and 4 h later the lower chamber media was withdrawn and used to treat H4βAPP695 wt cells for 24 h to assay inhibition of Aβ secretion. The concentration of each compound added to the upper chamber would lead to 10 μM in the lower chamber if equilibration was complete, and thus the activity of the lower chamber media was compared to the activity of 10 μM drug added directly to the H4βAPP695 wt cells. Table 4 shows the result of this study. Compound 1 crossed the monolayer and the bottom chamber media led to a 27% reduction in Aβ production, yielding 38% of the theoretical activity. The compound with the most activity in the lower chamber was compound 48, which decreased Aβ production by 50%, with 71% of its theoretical activity. The lower chamber media from the other two compounds that were highly active after incubation with hepatocytes, compounds 76 and 53, decreased Aβ secretion by 32% and 1%, respectively. On the basis of the combined activity data from these last two studies, compound 48 was selected as the lead drug for in vivo study.

TABLE 4
Epithelial Transcellular Transport of Drug Activity
transcellular transport^{a}

| | No total Aβ | Yes total Aβ | % theoretical |
|---|---|---|---|
| compd | (% decrease) | (% decrease) | activity^{b} |
| 1 | 69.4 | 26.7 | 38.4 |
| 27 | 60.6 | 17.9 | 29.5 |
| 48 | 70.5 | 49.9 | 70.8 |
| 52 | 57.8 | 26.5 | 45.9 |
| 53 | 84.4 | 1.1 | 1.3 |
| 65 | 51.6 | 31.0 | 60.1 |
| 73 | 67.7 | 2.7 | 4.0 |
| 76 | 75.6 | 31.6 | 41.8 |

^{a}Drugs alone or after incubation with Caco-2 cells to yield 10 μM if in equilibrium.
^{b}% of activity passing through Caco-2 monolayer.

### In Vivo Activity of Lead Drug

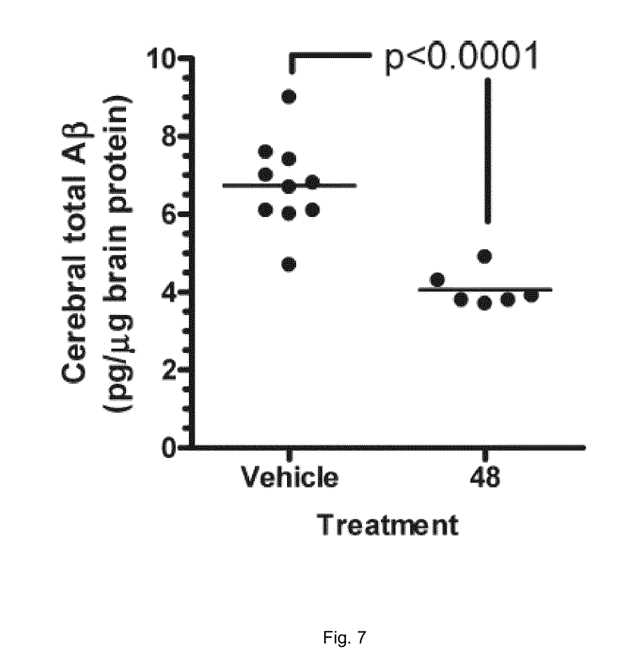

In APP transgenic mice, cerebral Aβ turns over rapidly prior to the onset of plaque formation with a half-life of about 2 h, allowing one to test Aβ lowering drugs acutely after a single administration. Thus, we administered a single high dose of our lead drug 48 (100 mg/kg) or vehicle iv to female APP transgenic mice and sacrificed the mice 6 h later for assay of cerebral Aβ levels. 48 led to a 39.6% decrease in cerebral Aβ levels (FIG. 7, p<0.0001).

### Example 2

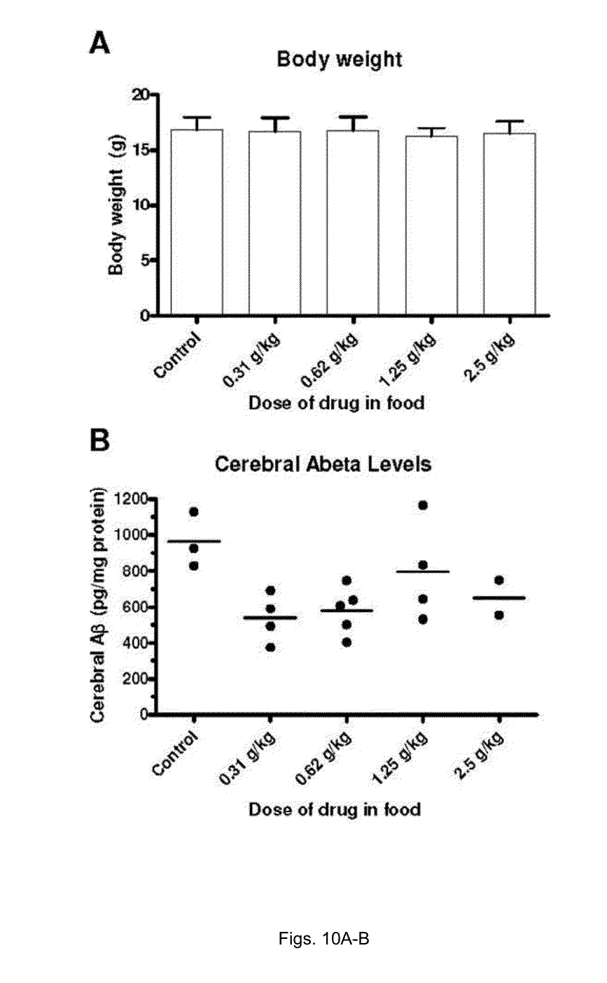

This Example show that oral treatment with compound 48 decreases cerebral Aβ levels in APP transgenic mice. In this Example, mutant APP/PS1 transgenic mice were obtained from Dr. Mathias Jucker (R Radde et al. 2006, EMBO Reports 7:940-946). Compound 48 was synthesized in a batch of about 25 g, and analytical methods showed >95% purity. The drug was milled into the chow diet (Harlan Teklad #2018) at 4 doses: 0.31, 0.62, 1.25, and 2.5 g/kg. Assuming each mouse eats 4 g of per day, the lowest dose should yield 1.24 mg intake per day, or about 75 mg/kg body weight. Six-week old female transgenic mice were fed ad librium with the chow diet or the diet supplemented with the four drug doses. Body weights were measured after the one-week feeding, and there was no significant effect of any drug dose on body weights (FIG. 10A). At the end of the study, brains were isolated, and half brains were homogenized in a buffer containing guanidine hydrochloride and SDS. This extract was diluted and used in assays for total protein and an ELISA for Abeta levels. The oral drug treatment led to decreased cerebral Aβ levels (FIG. 10B), with reductions ranging from 18% to 44% The lowest 2 doses were significantly different from the control by ANOVA with Dunnett's posttest, p<0.05. There was not a dose response in this study, as the effect appeared to plateau out even at the lowest dose of the drug, 0.31 g/kg diet. This oral diet study supported a prior finding using 3 oral gavages of this drug at an estimated dose of 200 mg/kg body weight delivered in Cremaphor over a 1.5 day period in which we observed a 30% decrease in cerebral Aβ levels (data not shown).

From the above description of the application, those skilled in the art will perceive improvements, changes and modifications. Such improvements, changes, and modifications are within the skill of those in the art and are intended to be covered by the appended claims. All patents, patent applications, and publication cited herein are incorporated by reference in their entirety.

Having described the invention, the following is claimed:

1. A pharmaceutical composition for inhibiting amyloid β peptide secretion in a subject, the composition comprising a compound having the formula (I): 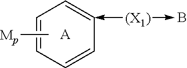
where M is selected from a substituted or unsubstituted alkyl, halo, alkoxy, aryl, cyclic, or heterocyclic group;
p is an integer from 0-3;
X₁ is a 3-9 atoms in length linker connecting A and B;
B is selected from a substituted or unsubstituted aryl, alkoxy or amine group; and
a pharmaceutically acceptable salt thereof; and a pharmaceutical carrier.

2. The pharmaceutical composition of claim 1, the compound comprising the formula (II): 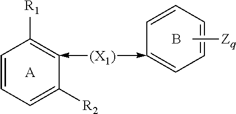
where R₁ and R₂ are individually selected from a substituted or unsubstituted, aryl, cyclic, or heterocyclic group;
X₁ is a 6-7 atoms in length linker connecting A and B;
Z is a substituted or unsubstituted alkyl, halo, alkoxy, or amine group;
q is an integer from 0-3; and pharmaceutically acceptable salts thereof.

3. The pharmaceutical composition of claim 2, X₁ comprising the following structure: 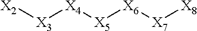
where X₂ is selected from CH₂ and CO;
X₃ is selected from NH, CH₂ and CO;
X₄ is selected from NH and CH₂;
X₅ is CH₂;
X₆ is selected from SH₂, SHO, SO₂ and CH₂;
X₇ is selected from SH₂, SHO, SO₂ and CH₂; and
X₈ is selected from nothing and CH₂.

4. The pharmaceutical composition of claim 2, where R₁ and R₂ are independently selected from H, Cl, CH₃, OH, NO₂, F, Br, CF₃, or an alkoxy group.

5. The pharmaceutical composition of claim 2, where Z is selected from Cl, alkoxy, OH, CN, C(NH₂)NOH, NO₂, NH₂, CO₂Et, COOH, CONH, F, CH₂, CHO, CF₃, BR, I, CONHC₃H₅, NHCOC₃H₅ and OCH₂CH₂OCH₂CH₂OH.

6. The pharmaceutical composition of claim 1, the compound is selected from the group consisting of: 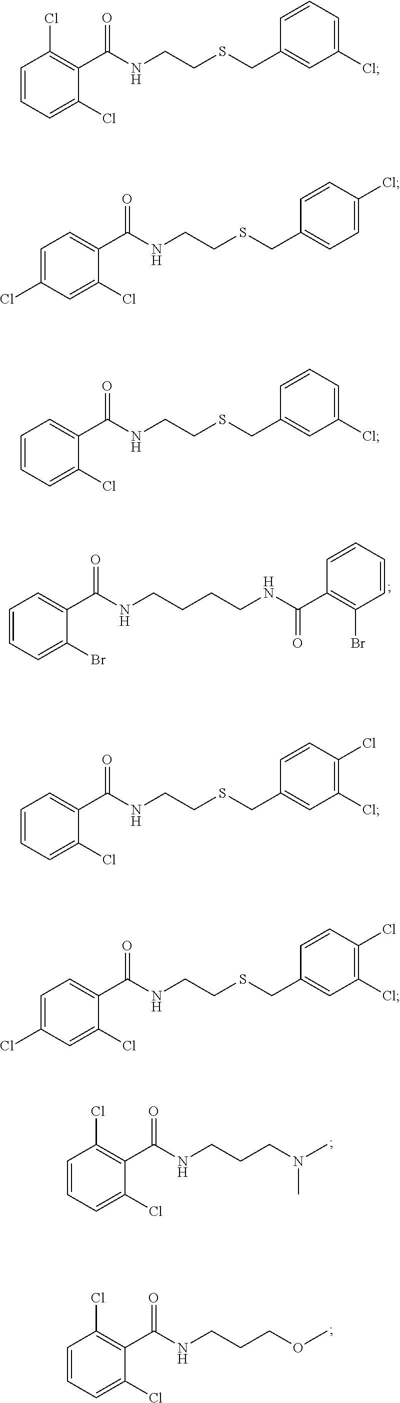 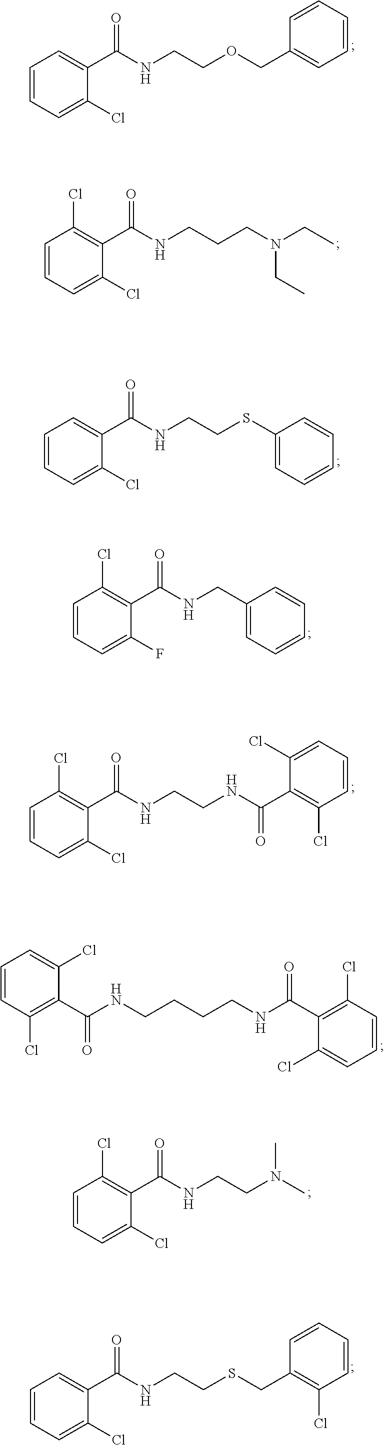 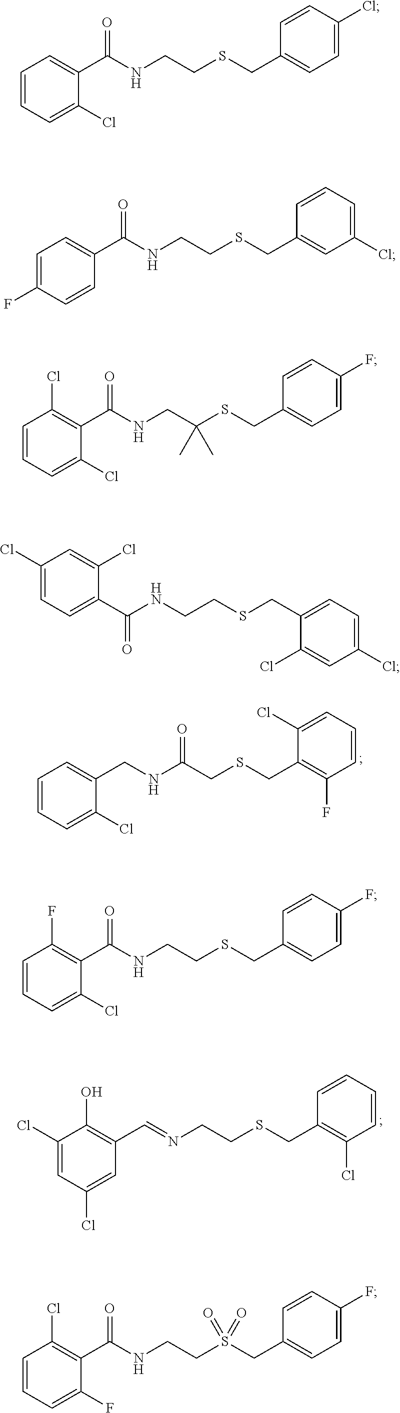 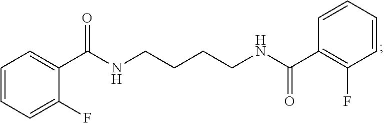 [SMILES: O=C(NCCCCNC(=O)c1ccccc1F)c1ccccc1F]
and pharmaceutically acceptable salts thereof.

7. The pharmaceutical composition of claim 1, the compound comprising: 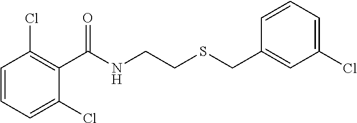 [SMILES: O=C(NCCSCc1cccc(Cl)c1)c1c(Cl)cccc1Cl]
and pharmaceutically acceptable salts thereof.

8. The pharmaceutical composition of claim 1, the compound is selected from the group consisting of: 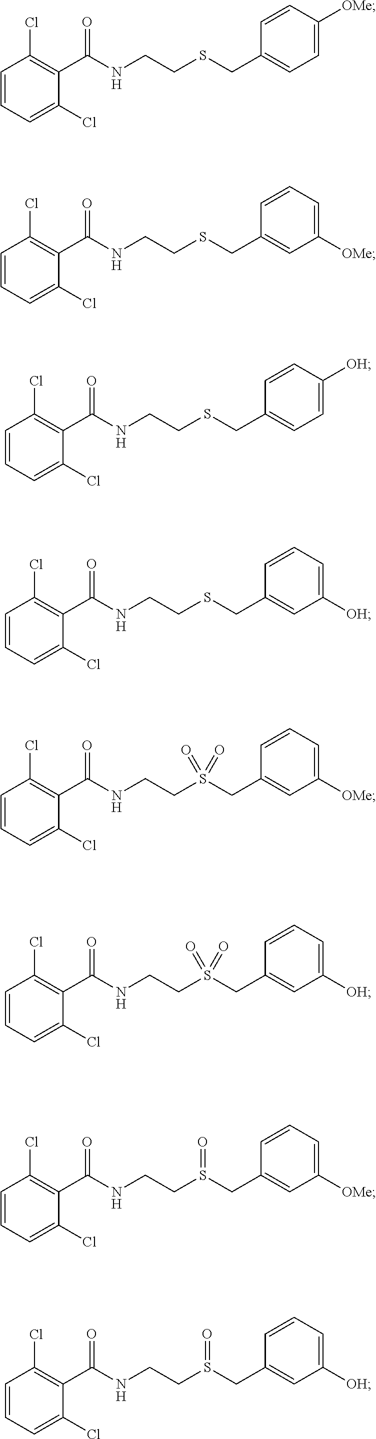 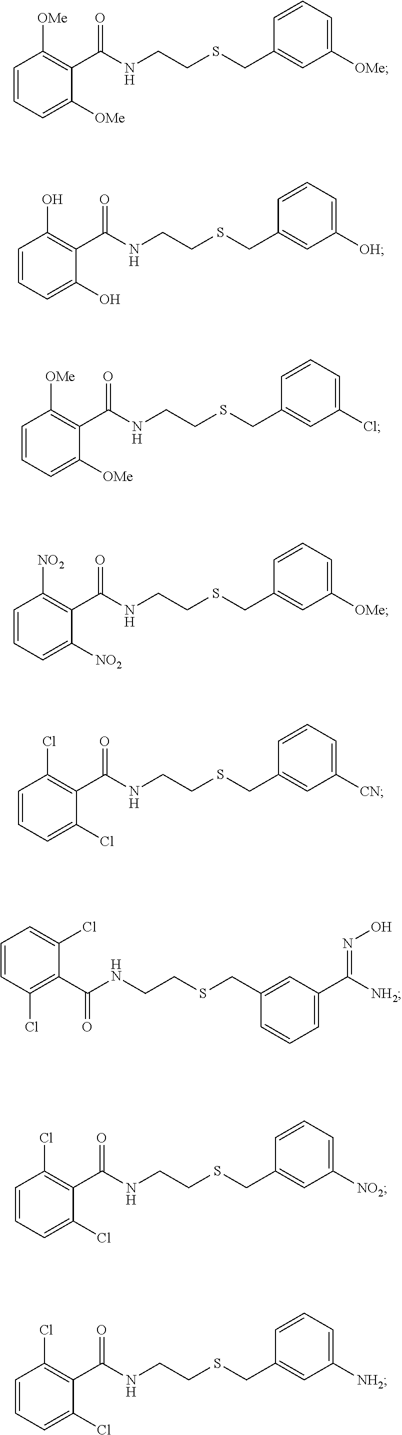 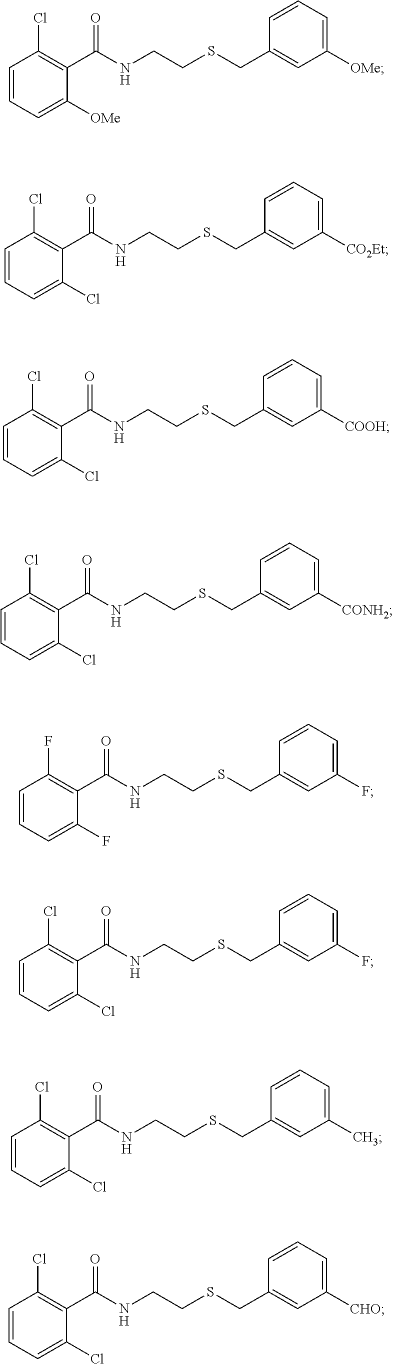 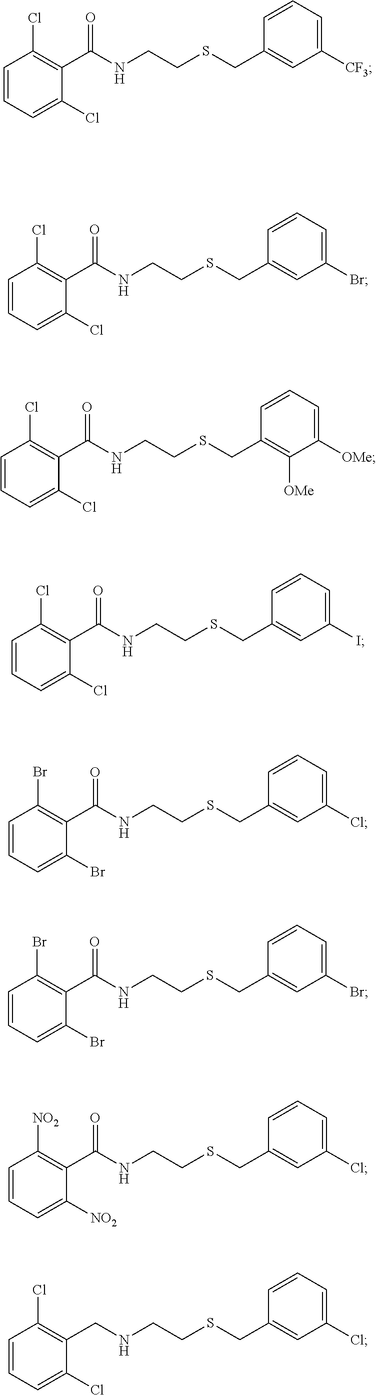 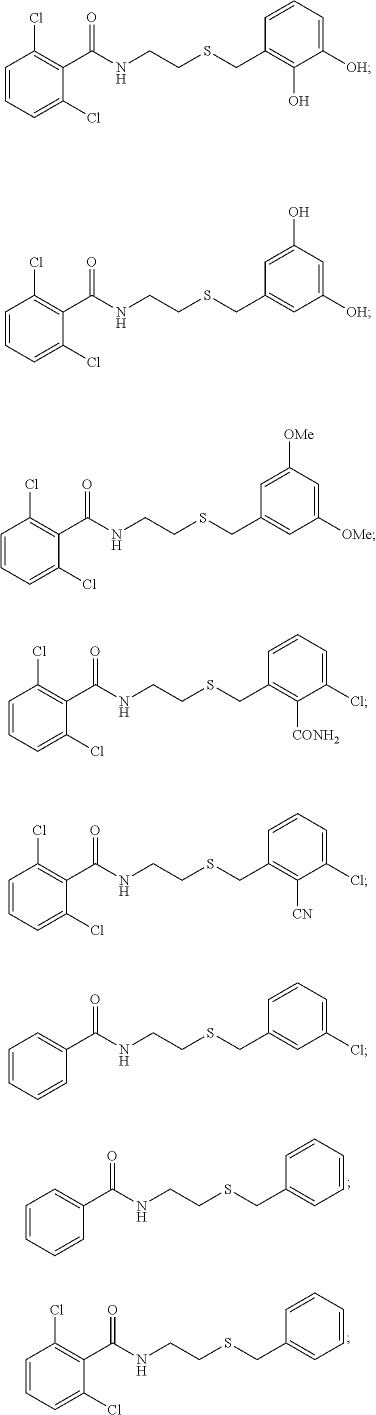 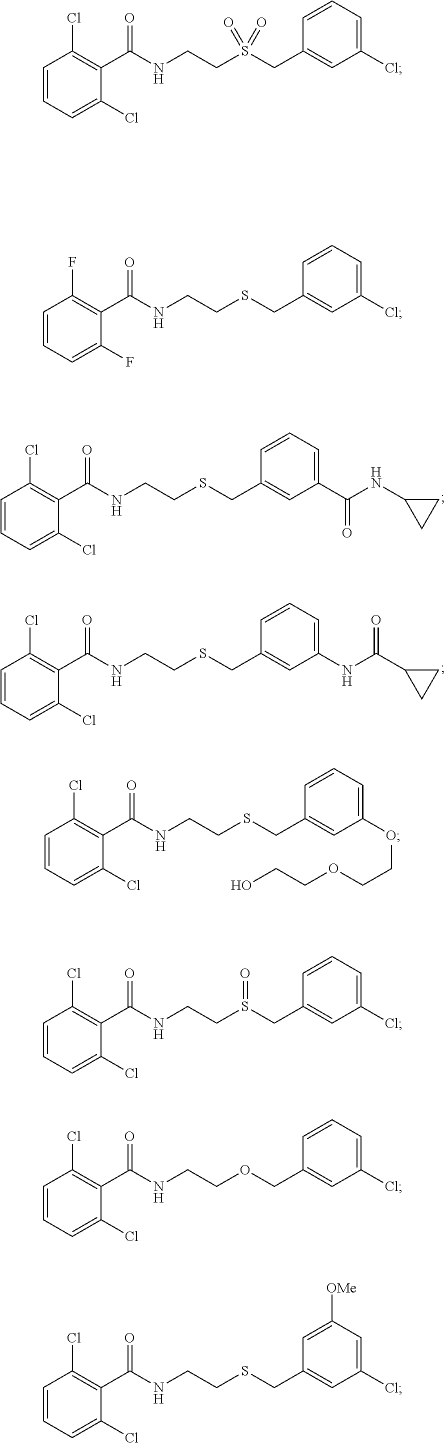 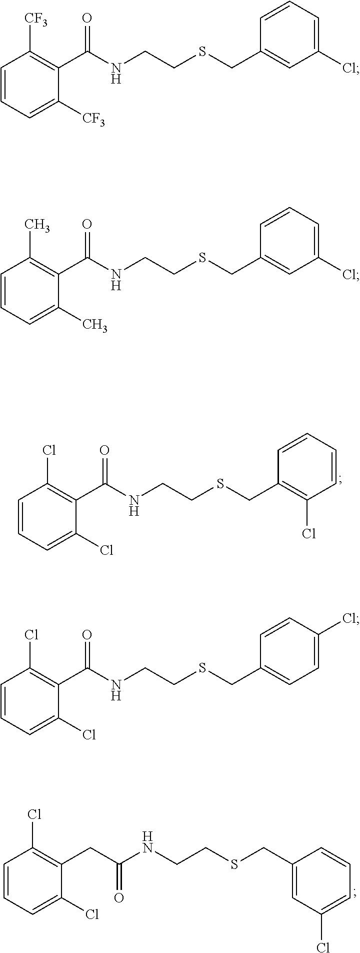 [SMILES: O=C(NCCSCc1cccc(Cl)c1)c1c(C(F)(F)F)cccc1C(F)(F)F ; Cc1cccc(C)c1C(=O)NCCSCc1cccc(Cl)c1 ; O=C(NCCSCc1ccccc1Cl)c1c(Cl)cccc1Cl ; O=C(NCCSCc1ccc(Cl)cc1)c1c(Cl)cccc1Cl ; O=C(Cc1c(Cl)cccc1Cl)NCCSCc1cccc(Cl)c1]
and pharmaceutically acceptable salts thereof.

9. A method for inhibiting amyloid β peptide secretion in a subject, the method comprising:
administering to the subject a therapeutically effective amount of a pharmaceutical composition, the pharmaceutical composition including a compound having the formula (I): 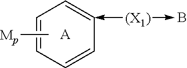
where M is selected from a substituted or unsubstituted alkyl, halo, alkoxy, aryl, cyclic, or heterocyclic group;
p is an integer from 0-3;
X₁ is a 3-9 atoms in length linker connecting A and B;
B is selected from a substituted or unsubstituted aryl, alkoxy or amine group; and
a pharmaceutically acceptable salt thereof; and a pharmaceutical carrier.

10. The method of claim 9, the compound comprising the formula (II): 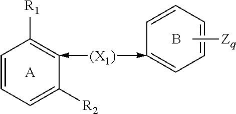
where R₁ and R₂ are individually selected from a substituted or unsubstituted, aryl, cyclic, or heterocyclic group;
X₁ is a 6-7 atoms in length linker connecting A and B;
Z is a substituted or unsubstituted alkyl, halo, alkoxy, or amine group;
q is an integer from 0-3; and pharmaceutically acceptable salts thereof.

11. The method of claim 10, X₁ comprising the following structure: 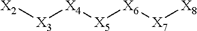
where X₂ is selected from CH₂ and CO;
X₃ is selected from NH, CH₂ and CO;
X₄ is selected from NH and CH₂;
X₅ is CH₂;
X₆ is selected from SH₂, SHO, SO₂ and CH₂;
X₇ is selected from SH₂, SHO, SO₂ and CH₂; and
X₈ is selected from nothing and CH₂.

12. The method of claim 10 where R₃ and R₄ are independently selected from H, Cl, CH₃, OH, NO₂, F, Br, CF₃ and an alkoxy group.

13. The method of claim 10, where Z is selected from Cl, alkoxy, OH, CN, C(NH₂)NOH, NO₂, NH₂, CO₂Et, COOH, CONH, F, CH₂, CHO, CF₃, BR, I, CONHC₃H₅, NHCOC₃H₅ and OCH₂CH₂OCH₂CH₂OH.

14. The method of claim 9, the compound is selected from the group consisting of: 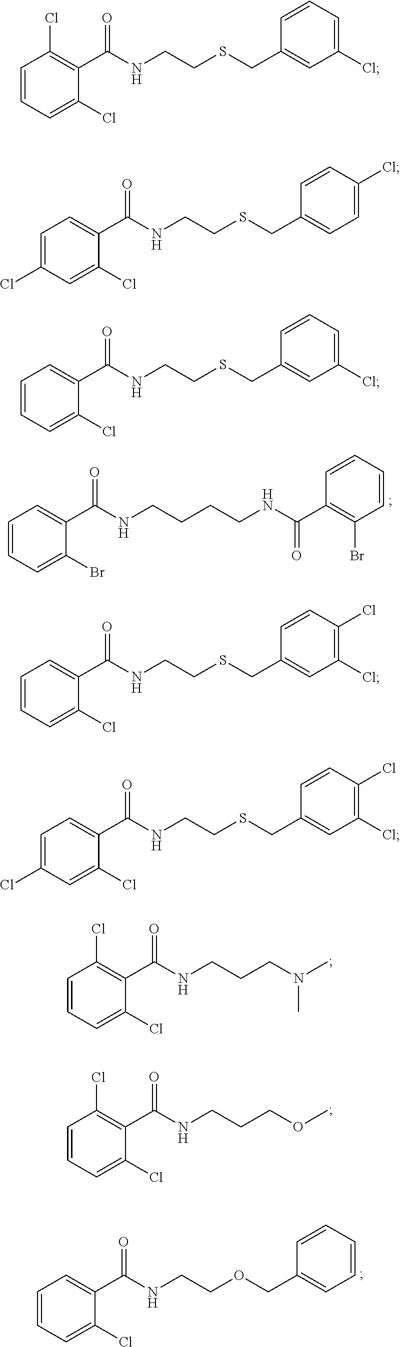 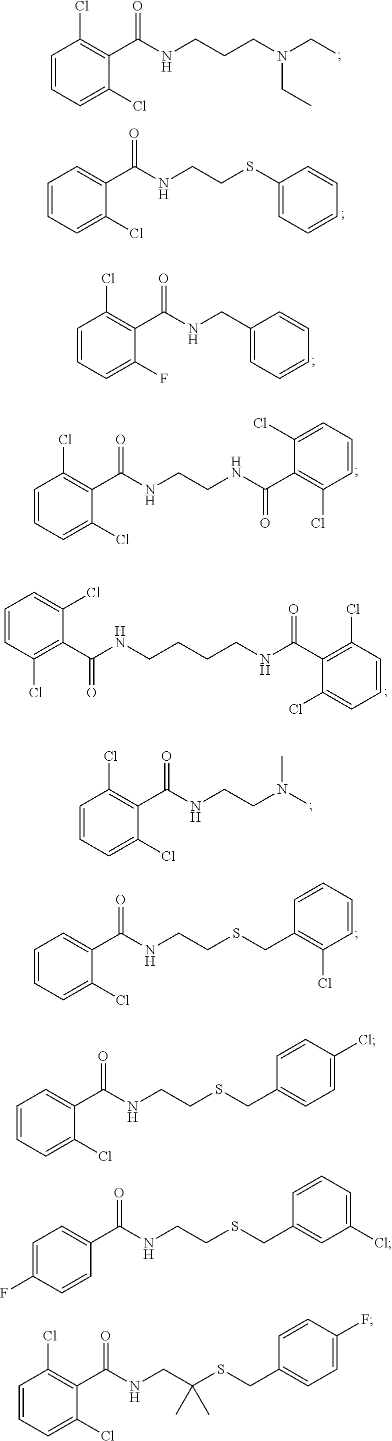 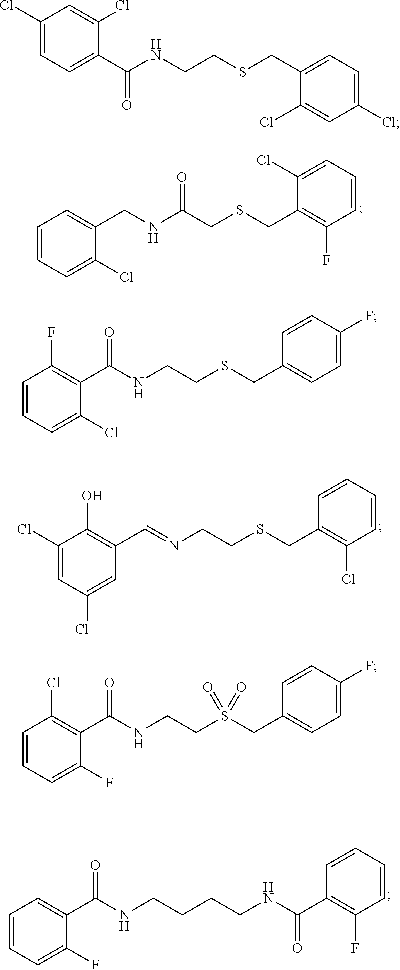
and pharmaceutically acceptable salts thereof.

15. The method of claim 9, the compound is selected from the group consisting of: 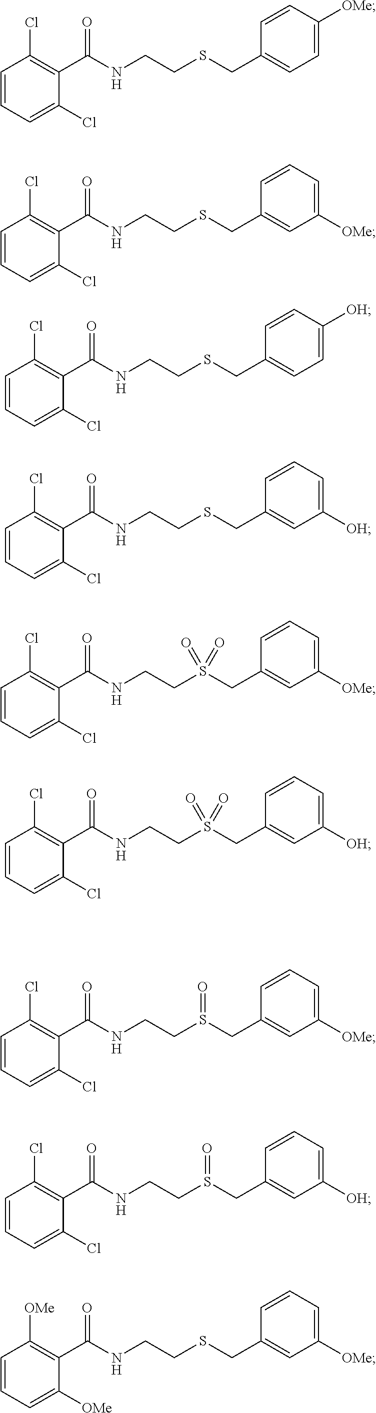 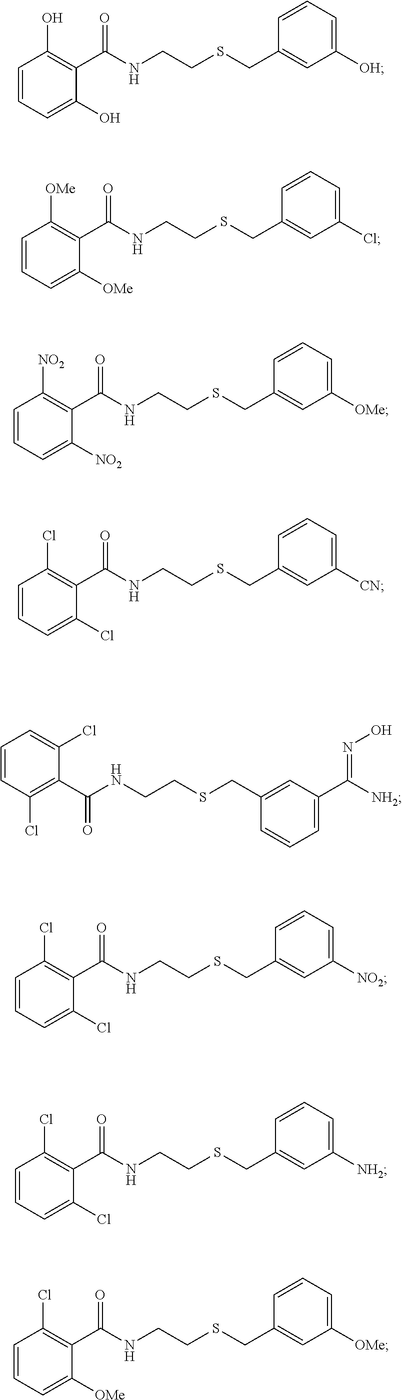 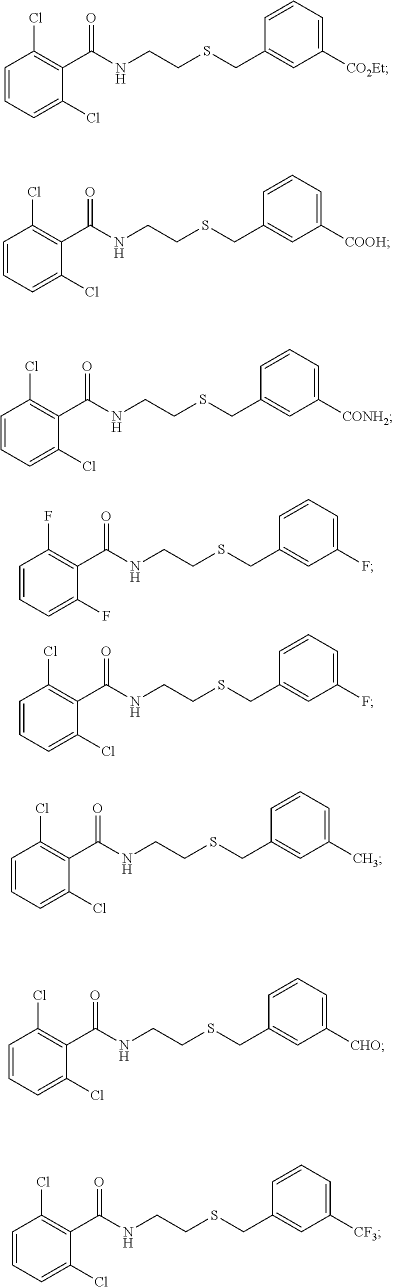 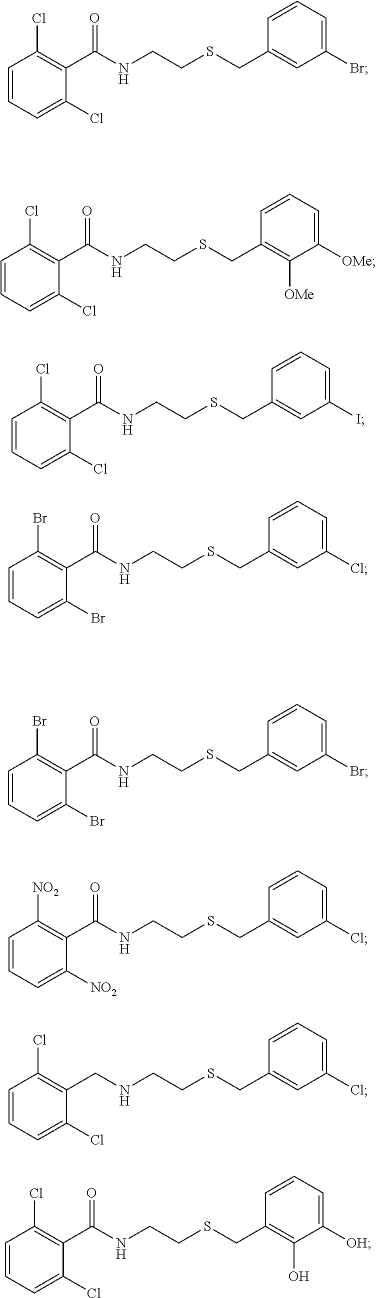 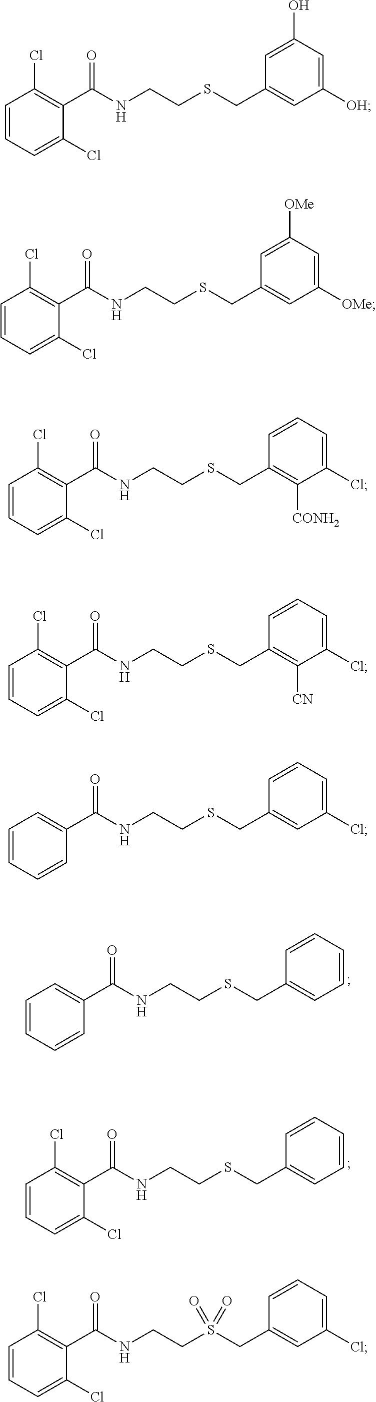 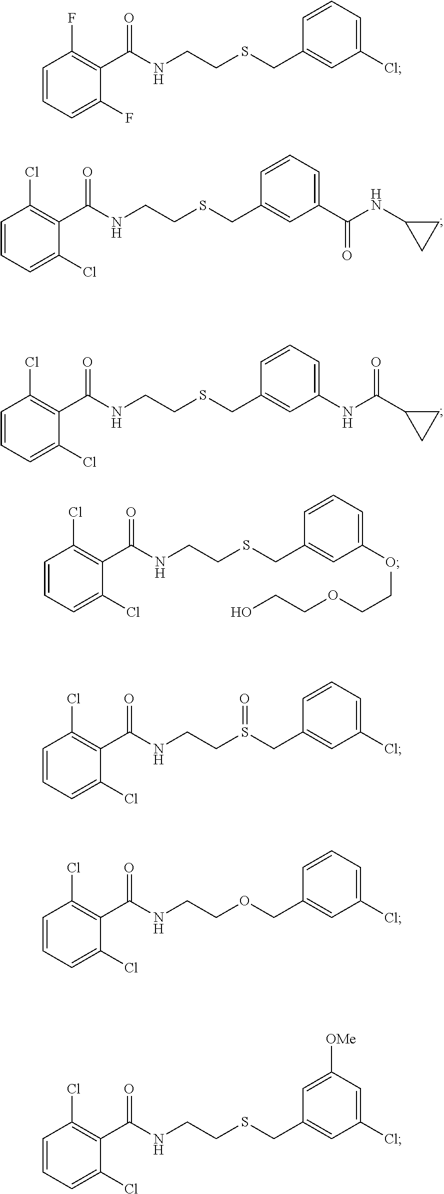 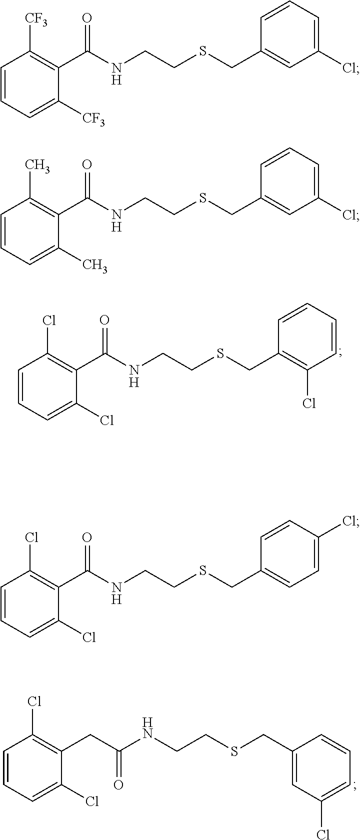 [SMILES: O=C(NCCSCc1cccc(Cl)c1)c1c(C(F)(F)F)cccc1C(F)(F)F ; Cc1cccc(C)c1C(=O)NCCSCc1cccc(Cl)c1 ; O=C(NCCSCc1ccccc1Cl)c1c(Cl)cccc1Cl ; O=C(NCCSCc1ccc(Cl)cc1)c1c(Cl)cccc1Cl ; O=C(Cc1c(Cl)cccc1Cl)NCCSCc1cccc(Cl)c1]
and pharmaceutically acceptable salts thereof.

16. A method for treating a neurological or neurodegenerative disorder in a subject, the method comprising:
administering to the subject a therapeutically effective amount of a pharmaceutical composition, the pharmaceutical composition including a compound having the formula (I): 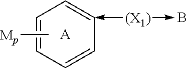
where M is selected from a substituted or unsubstituted alkyl, halo, alkoxy, aryl, cyclic, or heterocyclic group;
p is an integer from 0-3;
X₁ is a 3-9 atoms in length linker connecting A and B;
B is selected from a substituted or unsubstituted aryl, alkoxy or amine group; and
a pharmaceutically acceptable salt thereof; and a pharmaceutical carrier.

17. The method of claim 16, the compound comprising the formula (II): 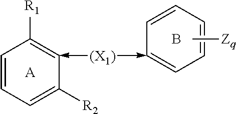
where R₁ and R₂ are individually selected from a substituted or unsubstituted, aryl, cyclic, or heterocyclic group;
X₁ is a 6-7 atoms in length linker connecting A and B;
Z is a substituted or unsubstituted alkyl, halo, alkoxy, or amine group;
q is an integer from 0-3; and pharmaceutically acceptable salts thereof.

18. The method of claim 17, X₁ comprising the following structure: 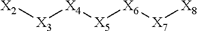
where X₂ is selected from CH₂ and CO;
X₃ is selected from NH, CH₂ and CO;
X₄ is selected from NH and CH₂;
X₅ is CH₂;
X₆ is selected from SH₂, SHO, SO₂ and CH₂;
X₇ is selected from SH₂, SHO, SO₂ and CH₂; and
X₈ is selected from nothing and CH₂.

19. The method of claim 17 where R₃ and R₄ are independently selected from H, Cl, CH₃, OH, NO₂, F, Br, CF₃ and an alkoxy group.

20. The method of claim 17, where Z is selected from Cl, alkoxy, OH, CN, C(NH₂)NOH, NO₂, NH₂, CO₂Et, COOH, CONH, F, CH₂, CHO, CF₃, BR, I, CONHC₃H₅, NHCOC₃H₅ and OCH₂CH₂OCH₂CH₂OH.

21. The method of claim 16, the compound is selected from the group consisting of: 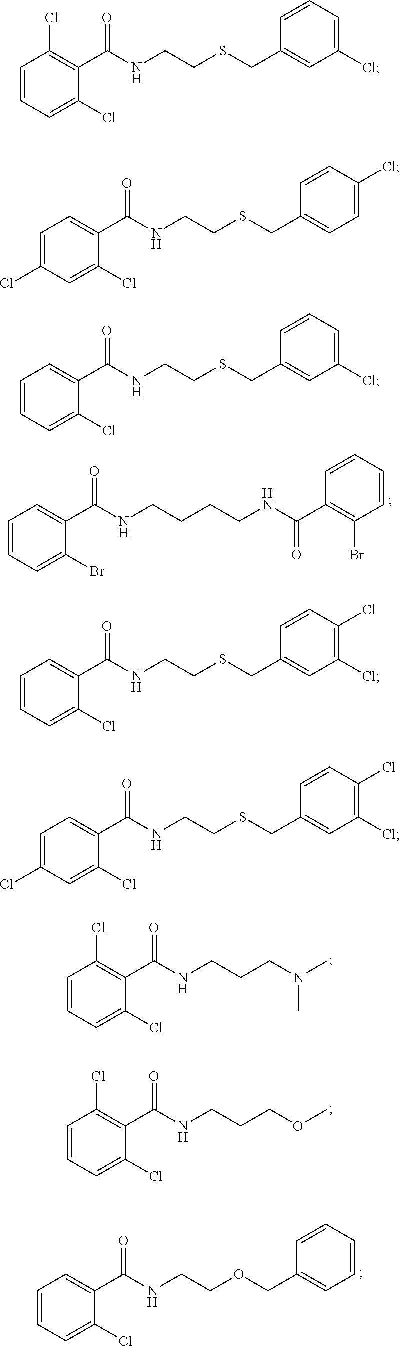 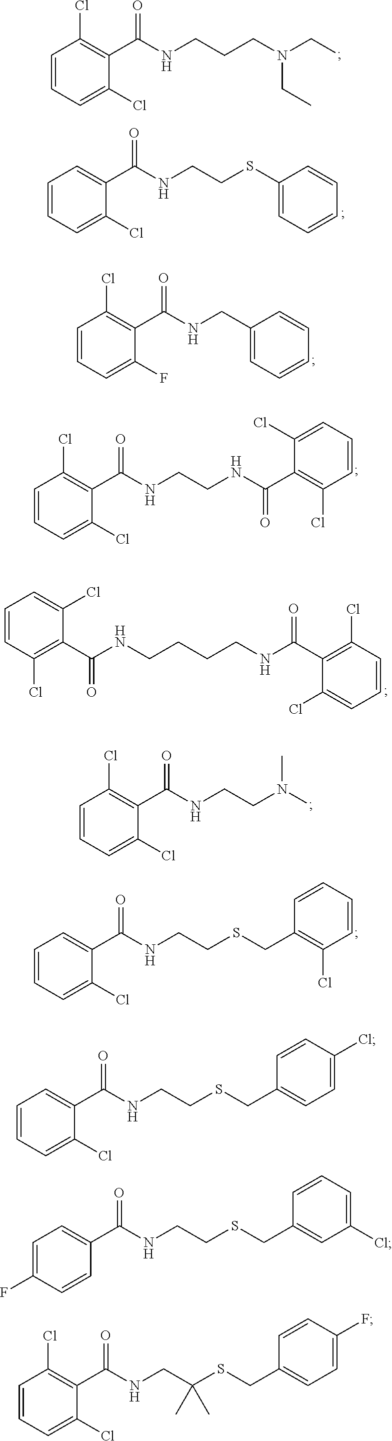 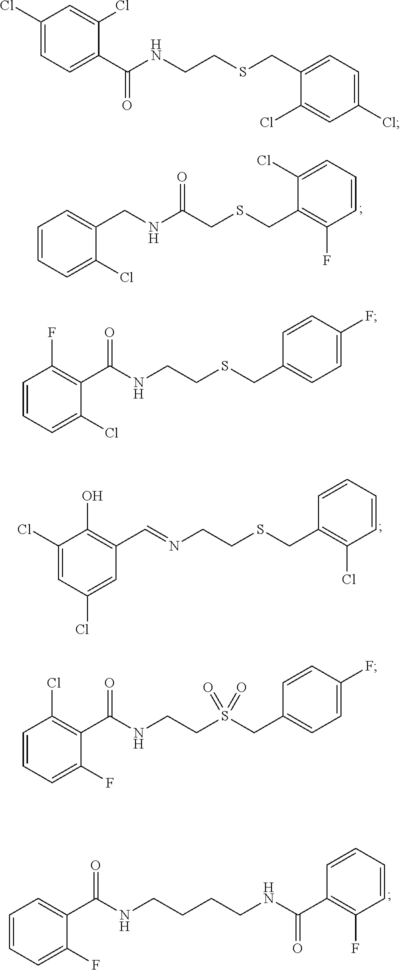
and pharmaceutically acceptable salts thereof.

22. The method of claim 16, the compound is selected from the group consisting of: 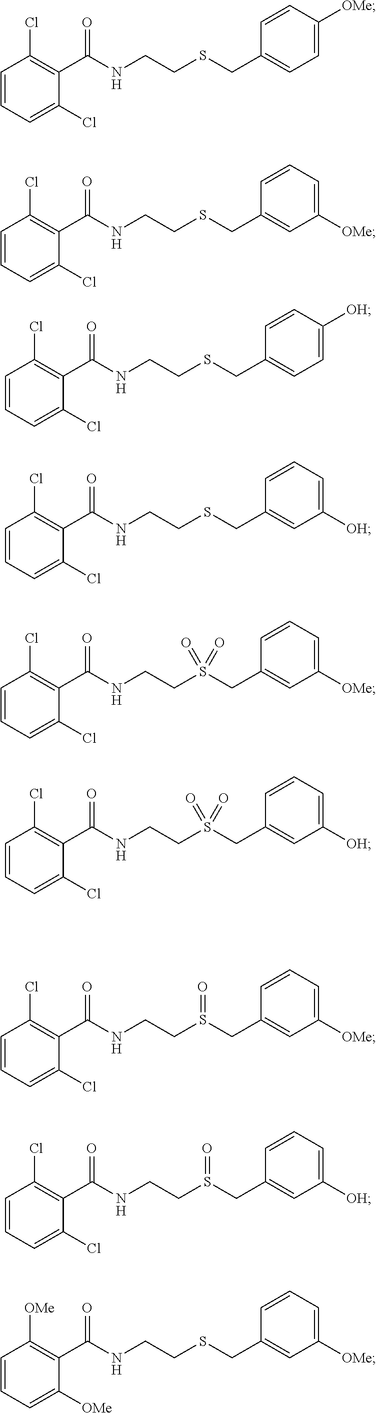 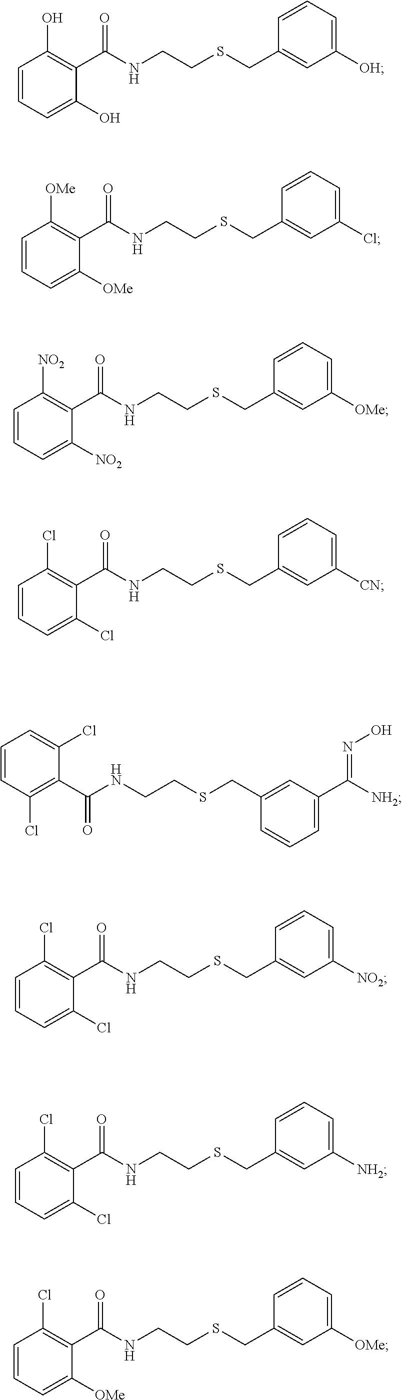 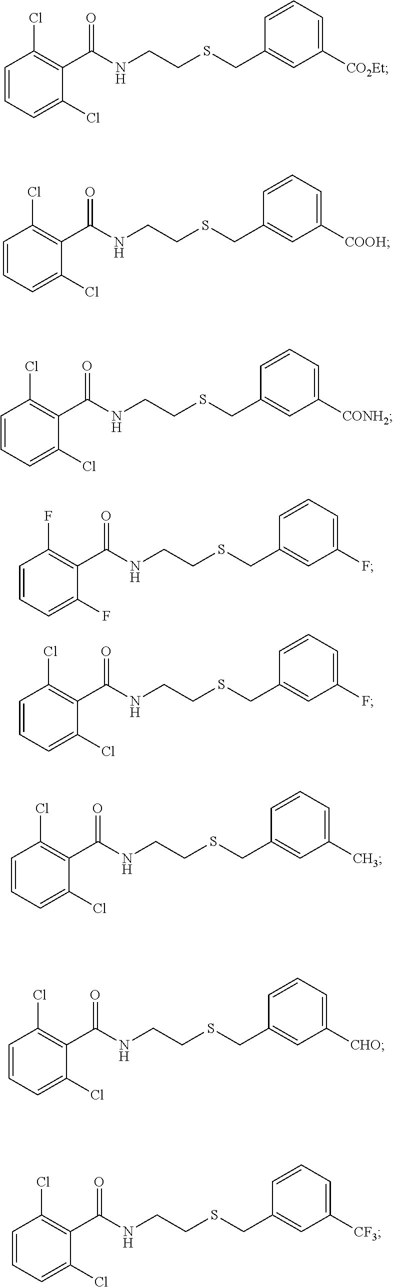 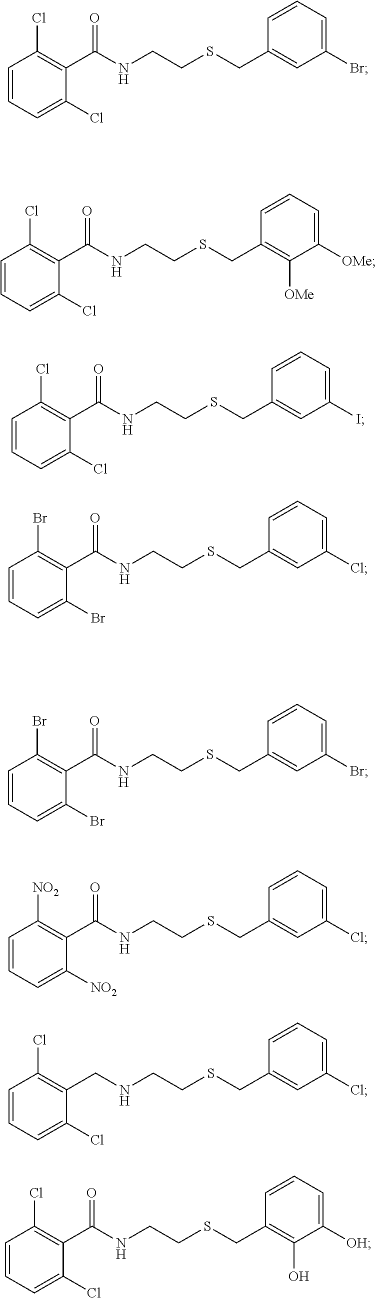 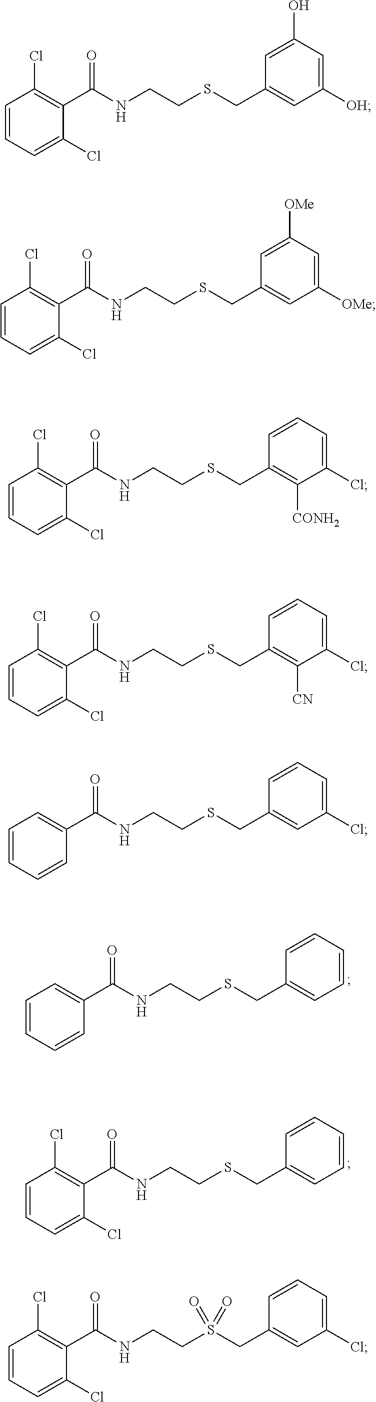 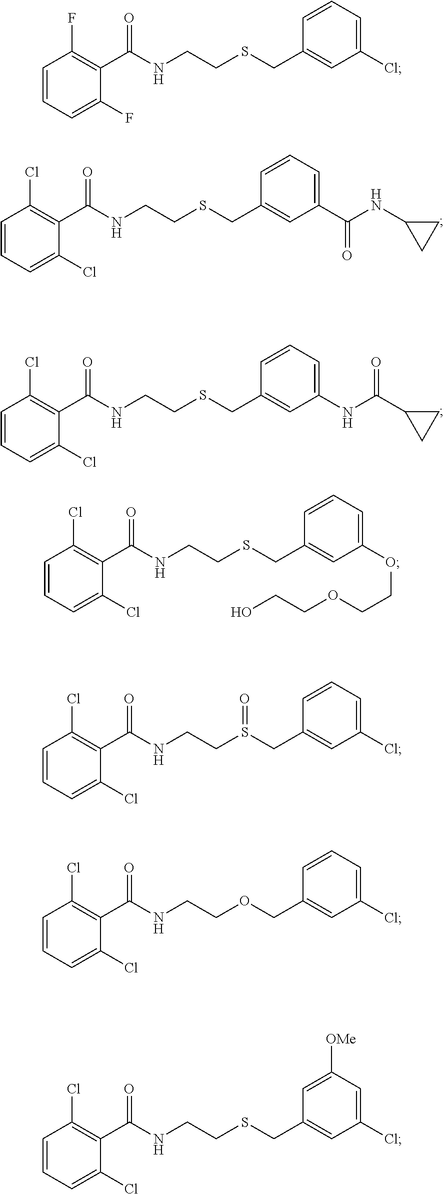 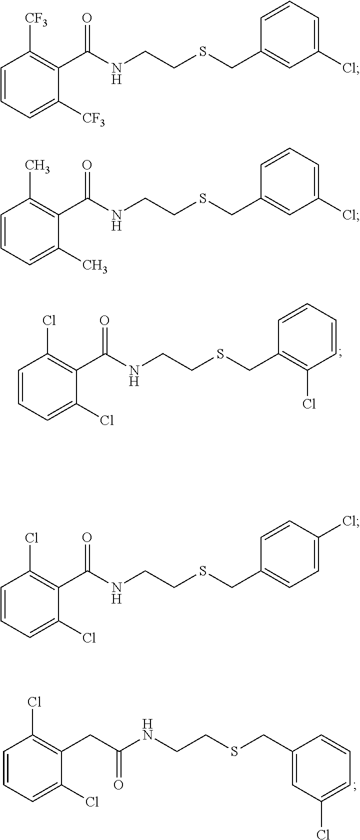 [SMILES: O=C(NCCSCc1cccc(Cl)c1)c1c(C(F)(F)F)cccc1C(F)(F)F ; Cc1cccc(C)c1C(=O)NCCSCc1cccc(Cl)c1 ; O=C(NCCSCc1ccccc1Cl)c1c(Cl)cccc1Cl ; O=C(NCCSCc1ccc(Cl)cc1)c1c(Cl)cccc1Cl ; O=C(Cc1c(Cl)cccc1Cl)NCCSCc1cccc(Cl)c1]
and pharmaceutically acceptable salts thereof.

23. The method of claim 16, the pharmaceutical composition promoting neuronal survival in the subject.
